SEQUENCE LISTING (908 sequences)

SEQ ID NO: 1 - DNA, 40 bp, Homo sapiens
tgctcatctgtgcacttctgttcaacctatcacactgagt

SEQ ID NO: 2 - DNA, 40 bp, Homo sapiens
aaaccgtttttcattattgctcctgaccccctctcatggg

SEQ ID NO: 3 - DNA, 40 bp, Homo sapiens
tgcacaggggaccttaaccagatcattagtttatatgcct

SEQ ID NO: 4 - DNA, 40 bp, Homo sapiens
cacacactccagaacagatggtatccagatgccttatggg

SEQ ID NO: 5 - DNA, 40 bp, Homo sapiens
gcgaaccatttctaatgttctgatttttcagagccagcca

SEQ ID NO: 6 - DNA, 40 bp, Homo sapiens
tgtgggatccgtctcagttactttatagccatacctggta

SEQ ID NO: 7 - DNA, 40 bp, Homo sapiens
agctgaatggtgatggtgtgaagtataggttaaattgggt

SEQ ID NO: 8 - DNA, 40 bp, Homo sapiens
gtgcattgctgttgcattgcacgtgtgtgaggcgggtgca

SEQ ID NO: 9 - DNA, 40 bp, Homo sapiens
aaagctgtagggcctccaggttctcaagctgtgagtggaa

SEQ ID NO: 10 - DNA, 40 bp, Homo sapiens
tggttgacatatggctgctaatgccctcctttctagtggg

SEQ ID NO: 11 - DNA, 40 bp, Homo sapiens
gtgtgcgtaacggctggtgtgtttctctagctgagctaat

SEQ ID NO: 12 - DNA, 40 bp, Homo sapiens
acctgctatgccaacatattgccatctttcctgtctgaca

SEQ ID NO: 13 - DNA, 40 bp, Homo sapiens
acagtgagtgcgagtattatttcttgccagcgggtggaag

SEQ ID NO: 14 - DNA, 40 bp, Homo sapiens
acactgctcgctctatgttaattttagctcttcccctgga

SEQ ID NO: 15 - RNA, 73 nt, Homo sapiens
cagggugugugacugguugaccagaggggcaugcacuguguucacccugugggccaccuagucaccaacccuc

SEQ ID NO: 16 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 17 - RNA, 85 nt, Homo sapiens
aggccucucucuccguguucacagcggaccuugauuuaaauguccauacaauuaaggcacgcggugaaugccaagaauggggcug

SEQ ID NO: 18 - RNA, 95 nt, Homo sapiens
uuguaccuggugugauuauaaagcaaugagacugauugucauaugucguuugugggauccgucucaguuacuuuauagccauaccugguaucuua

SEQ ID NO: 19 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 20 - RNA, 96 nt, Homo sapiens
gcgggcggccccgcggugcauugcuguugcauugcacgugugugaggcgggugcagugccucggcagugcagcccggagccggccccuggcaccac

SEQ ID NO: 21 - RNA, 94 nt, Homo sapiens
cggggcggccgcucucccuguccuccaggagcucacgugugccugccugugagcgccucgacgacagagccggcgccugccccagugucugcgc

SEQ ID NO: 22 - RNA, 52 nt, Homo sapiens
gcagcaaggaaggcagggguccuaagguguguccuccugcccuccuugcugu

SEQ ID NO: 23 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 24 - DNA, 71 bp, Homo sapiens
ggagaggaggcaagaugcuggcauagcuguugaacugggaaccugcuaugccaacauauugccaucuuucc

SEQ ID NO: 25 - RNA, 97 nt, Homo sapiens
ccuagaauguuauuaggucggugcaaaaguaauugcgaguuuuaccauuacuuucaauggcaaaacuggcaauuacuuuugcaccaacguaauacuu

SEQ ID NO: 26 - RNA, 80 nt, Homo sapiens
cucuaggaugugcucauugcaugggcuguguauaguauuauucaauacccagagcaugcagugugaacauaauagagauu

SEQ ID NO: 27 - DNA, 22 bp, Homo sapiens
atacatacttctttacattcca

SEQ ID NO: 28 - DNA, 22 bp, Homo sapiens
acacaaattcggttctacaggg

SEQ ID NO: 29 - DNA, 21 bp, Homo sapiens
gccaatatttctgtgctgcta

SEQ ID NO: 30 - DNA, 22 bp, Homo sapiens
acagctggttgaaggggaccaa

SEQ ID NO: 31 - DNA, 22 bp, Homo sapiens
cacataggaatgaaaagccata

SEQ ID NO: 32 - DNA, 22 bp, Homo sapiens
tgtgagttctaccattgccaaa

SEQ ID NO: 33 - DNA, 20 bp, Homo sapiens
tccagtcaaggatgtttaca

SEQ ID NO: 34 - DNA, 21 bp, Homo sapiens
cacaagatcggatctacgggt

SEQ ID NO: 35 - RNA, 85 nt, Homo sapiens
accuacucagaguacauacuucuuuauguacccauaugaacauacaaugcuauggaauguaaagaaguauguauuuuugguaggc

SEQ ID NO: 36 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 37 - RNA, 87 nt, Homo sapiens
agcuucccuggcucuagcagcacagaaauauuggcacagggaagcgagucugccaauauuggcugugcugcuccaggcaggguggug

SEQ ID NO: 38 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 39 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 40 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 41 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 42 - RNA, 81 nt, Homo sapiens
cccauuggcauaaacccguagauccgaucuuguggugaaguggaccgcacaagcucgcuucuaugggucugugucagugug

SEQ ID NO: 43 - DNA, 25 bp, Homo sapiens
aagatcattgctcctcctgagcgca

SEQ ID NO: 44 - DNA, 25 bp, Homo sapiens
cctcctgagcgcaagtactccgtgt

SEQ ID NO: 45 - DNA, 25 bp, Homo sapiens
tccgtgtggatcggcggctccatcc

SEQ ID NO: 46 - DNA, 25 bp, Homo sapiens
cagatgtggatcagcaagcaggagt

SEQ ID NO: 47 - DNA, 25 bp, Homo sapiens
gtccaccgcaaatgcttctaggcgg

SEQ ID NO: 48 - DNA, 25 bp, Homo sapiens
accacggccgagcgggaaatcgtgc

SEQ ID NO: 49 - DNA, 25 bp, Homo sapiens
ctgtgctacgtcgccctggacttcg

SEQ ID NO: 50 - DNA, 25 bp, Homo sapiens
gagcaagagatggccacggctgctt

SEQ ID NO: 51 - DNA, 25 bp, Homo sapiens
tcctccctggagaagagctacgagc

SEQ ID NO: 52 - DNA, 25 bp, Homo sapiens
ctgcctgacggccaggtcatcacca

SEQ ID NO: 53 - DNA, 25 bp, Homo sapiens
caggtcatcaccattggcaatgagc

SEQ ID NO: 54 - DNA, 25 bp, Homo sapiens
cggttccgctgccctgaggcactct

SEQ ID NO: 55 - DNA, 25 bp, Homo sapiens
cctgaggcactcttccagccttcct

SEQ ID NO: 56 - DNA, 25 bp, Homo sapiens
gagtcctgtggcatccacgaaacta

SEQ ID NO: 57 - DNA, 25 bp, Homo sapiens
atccacgaaactaccttcaactcca

SEQ ID NO: 58 - DNA, 25 bp, Homo sapiens
aactccatcatgaagtgtgacgtgg

SEQ ID NO: 59 - DNA, 25 bp, Homo sapiens
gacatccgcaaagacctgtacgcca

SEQ ID NO: 60 - DNA, 25 bp, Homo sapiens
aacacagtgctgtctggcggcacca

SEQ ID NO: 61 - DNA, 25 bp, Homo sapiens
accatgtaccctggcattgccgaca

SEQ ID NO: 62 - DNA, 25 bp, Homo sapiens
cagaaggagatcactgccctggcac

SEQ ID NO: 63 - DNA, 25 bp, Homo sapiens
agattcgggcaagtccaccactact

SEQ ID NO: 64 - DNA, 25 bp, Homo sapiens
ttcgggcaagtccaccactactggc

SEQ ID NO: 65 - DNA, 25 bp, Homo sapiens
caccactactggccatctgatctat

SEQ ID NO: 66 - DNA, 25 bp, Homo sapiens
ccatctgatctataaatgcggtggc

SEQ ID NO: 67 - DNA, 25 bp, Homo sapiens
tctgatctataaatgcggtggcatc

SEQ ID NO: 68 - DNA, 25 bp, Homo sapiens
tgcctgggtcttggataaactgaaa

SEQ ID NO: 69 - DNA, 25 bp, Homo sapiens
tgaaagctgagcgtgaacgtggtat

SEQ ID NO: 70 - DNA, 25 bp, Homo sapiens
cgtgaacgtggtatcaccattgata

SEQ ID NO: 71 - DNA, 25 bp, Homo sapiens
gaacgtggtatcaccattgatatct

SEQ ID NO: 72 - DNA, 25 bp, Homo sapiens
gtggtatcaccattgatatctcctt

SEQ ID NO: 73 - DNA, 25 bp, Homo sapiens
tatcaccattgatatctccttgtgg

SEQ ID NO: 74 - DNA, 25 bp, Homo sapiens
ccattgatatctccttgtggaaatt

SEQ ID NO: 75 - DNA, 25 bp, Homo sapiens
gtactatgtgactatcattgatgcc

SEQ ID NO: 76 - DNA, 25 bp, Homo sapiens
ctatgtgactatcattgatgcccca

SEQ ID NO: 77 - DNA, 25 bp, Homo sapiens
ctcatatcaacattgtcgtcattgg

SEQ ID NO: 78 - DNA, 25 bp, Homo sapiens
tatcaacattgtcgtcattggacac

SEQ ID NO: 79 - DNA, 25 bp, Homo sapiens
cattgtcgtcattggacacgtagat

SEQ ID NO: 80 - DNA, 25 bp, Homo sapiens
tgtcgtcattggacacgtagattcg

SEQ ID NO: 81 - DNA, 25 bp, Homo sapiens
cgtcattggacacgtagattcgggc

SEQ ID NO: 82 - DNA, 25 bp, Homo sapiens
gggtcagaaggattcctatgtgggc

SEQ ID NO: 83 - DNA, 25 bp, Homo sapiens
gaaggattcctatgtgggcgacgag

SEQ ID NO: 84 - DNA, 25 bp, Homo sapiens
ccccatcgagcacggcatcgtcacc

SEQ ID NO: 85 - DNA, 25 bp, Homo sapiens
cgtcaccaactgggacgacatggag

SEQ ID NO: 86 - DNA, 25 bp, Homo sapiens
caccttctacaatgagctgcgtgtg

SEQ ID NO: 87 - DNA, 25 bp, Homo sapiens
tcccgaggagcaccccgtgctgctg

SEQ ID NO: 88 - DNA, 25 bp, Homo sapiens
ggccaaccgcgagaagatgacccag

SEQ ID NO: 89 - DNA, 25 bp, Homo sapiens
ccagatcatgtttgagaccttcaac

SEQ ID NO: 90 - DNA, 25 bp, Homo sapiens
cccagccatgtacgttgctatccag

SEQ ID NO: 91 - DNA, 25 bp, Homo sapiens
cgttgctatccaggctgtgctatcc

SEQ ID NO: 92 - DNA, 25 bp, Homo sampiens
ggctgtgctatccctgtacgcctct

SEQ ID NO: 93 - DNA, 25 bp, Homo sapiens
cgcctctggccgtaccactggcatc

SEQ ID NO: 94 - DNA, 25 bp, Homo sapiens
taccactggcatcgtgatggactcc

SEQ ID NO: 95 - DNA, 25 bp, Homo sapiens
cggtgacggggtcacccacactgtg

SEQ ID NO: 96 - DNA, 25 bp, Homo sapiens
ccacactgtgcccatctacgagggg

SEQ ID NO: 97 - DNA, 25 bp, Homo sapiens
gcccatctacgaggggtatgccctc

SEQ ID NO: 98 - DNA, 25 bp, Homo sapiens
tgccatcctgcgtctggacctggct

SEQ ID NO: 99 - DNA, 25 bp, Homo sapiens
tgatatcgccgcgctcgtcgtcgac

SEQ ID NO: 100 - DNA, 25 bp, Homo sapiens
cgtcgtcgacaacggctccggcatg

SEQ ID NO: 101 - DNA, 25 bp, Homo sapiens
cggctccggcatgtgcaaggccggc

SEQ ID NO: 102 - DNA, 25 bp, Homo sapiens
accctcctaatagtcatactagtag

SEQ ID NO: 103 - DNA, 25 bp, Homo sapiens
ctaatagtcatactagtagtcatac

SEQ ID NO: 104 - DNA, 25 bp, Homo sapiens
gtcatactagtagtcatactccctg

SEQ ID NO: 105 - DNA, 25 bp, Homo sapiens
ctagtagtcatactccctggtgtag

SEQ ID NO: 106 - DNA, 25 bp, Homo sapiens
atgcagccagccatcaaatagtgaa

SEQ ID NO: 107 - DNA, 25 bp, Homo sapiens
tagtgaatggtctctctttggctgg

SEQ ID NO: 108 - DNA, 25 bp, Homo sapiens
taacccatgaaggataaaagcccca

SEQ ID NO: 109 - DNA, 25 bp, Homo sapiens
atagcactaatgctttaagatttgg

SEQ ID NO: 110 - DNA, 25 bp, Homo sapiens
ctttaagatttggtcacactctcac

SEQ ID NO: 111 - DNA, 25 bp, Homo sapiens
gatttggtcacactctcacctaggt

SEQ ID NO: 112 - DNA, 25 bp, Homo sapiens
cattgagccagtggtgctaaatgct

SEQ ID NO: 113 - DNA, 25 bp, Homo sapiens
ggtgctaaatgctacatactccaac

SEQ ID NO: 114 - DNA, 25 bp, Homo sapiens
tacatactccaactgaaatgttaag

SEQ ID NO: 115 - DNA, 25 bp, Homo sapiens
ctccaactgaaatgttaaggaagaa

SEQ ID NO: 116 - DNA, 25 bp, Homo sapiens
aacacaggagattccagtctacttg

SEQ ID NO: 117 - DNA, 25 bp, Homo sapiens
gcataatacagaagtcccctctact

SEQ ID NO: 118 - DNA, 25 bp, Homo sapiens
gtaacctgaactaatctgatgttaa

SEQ ID NO: 119 - DNA, 25 bp, Homo sapiens
aatctgatgttaaccaatgtattta

SEQ ID NO: 120 - DNA, 25 bp, Homo sapiens
ctgtttccttgttccaatttgacaa

SEQ ID NO: 121 - DNA, 25 bp, Homo sapiens
gctatcactgtacttgtagagtggt

SEQ ID NO: 122 - DNA, 25 bp, Homo sapiens
gcgcctggtcaccagggctgctttt

SEQ ID NO: 123 - DNA, 25 bp, Homo sapiens
ggtcaccagggctgcttttaactct

SEQ ID NO: 124 - DNA, 25 bp, Homo sapiens
tgcttttaactctggtaaagtggat

SEQ ID NO: 125 - DNA, 25 bp, Homo sapiens
ggatattgttgccatcaatgacccc

SEQ ID NO: 126 - DNA, 25 bp, Homo sapiens
catcaatgaccccttcattgacctc

SEQ ID NO: 127 - DNA, 25 bp, Homo sapiens
cttcattgacctcaactacatggtt

SEQ ID NO: 128 - DNA, 25 bp, Homo sapiens
caactacatggtttacatgttccaa

SEQ ID NO: 129 - DNA, 25 bp, Homo sapiens
ggtttacatgttccaatatgattcc

SEQ ID NO: 130 - DNA, 25 bp, Homo sapiens
ccaatatgattccacccatggcaaa

SEQ ID NO: 131 - DNA, 25 bp, Homo sapiens
tgattccacccatggcaaattccat

SEQ ID NO: 132 - DNA, 25 bp, Homo sapiens
attccatggcaccgtcaaggctgag

SEQ ID NO: 133 - DNA, 25 bp, Homo sapiens
tggcaccgtcaaggctgagaacggg

SEQ ID NO: 134 - DNA, 25 bp, Homo sapiens
catcaatggaaatcccatcaccatc

SEQ ID NO: 135 - DNA, 25 bp, Homo sapiens
tcccatcaccatcttccaggagcga

SEQ ID NO: 136 - DNA, 25 bp, Homo sapiens
cttccaggagcgagatccctccaaa

SEQ ID NO: 137 - DNA, 25 bp, Homo sapiens
gcgagatccctccaaaatcaagtgg

SEQ ID NO: 138 - DNA, 25 bp, Homo sapiens
cgatgctggcgctgagtacgtcgtg

SEQ ID NO: 139 - DNA, 25 bp, Homo sapiens
cgtggagtccactggcgtcttcacc

SEQ ID NO: 140 - DNA, 25 bp, Homo sapiens
cttcaccaccatggagaaggctggg

SEQ ID NO: 141 - DNA, 25 bp, Homo sapiens
cggatttggtcgtattgggcgcctg

SEQ ID NO: 142 - DNA, 25 bp, Homo sapiens
tcctcctgagcgcaagtactccgtg

SEQ ID NO: 143 - DNA, 25 bp, Homo sapiens
tgagcgcaagtactccgtgtggatc

SEQ ID NO: 144 - DNA, 25 bp, Homo sapiens
cttccagcagatgtggatcagcaag

SEQ ID NO: 145 - DNA, 25 bp, Homo sapiens
gtggatcagcaagcaggagtatgac

SEQ ID NO: 146 - DNA, 25 bp, Homo sapiens
ccgcaaatgcttctaggcggactat

SEQ ID NO: 147 - DNA, 25 bp, Homo sapiens
atgcttctaggcggactatgactta

SEQ ID NO: 148 - DNA, 25 bp, Homo sapiens
taacttgcgcagaaaacaagatgag

SEQ ID NO: 149 - DNA, 25 bp, Homo sapiens
cagcagtcggttggagcgagcatcc

SEQ ID NO: 150 - DNA, 25 bp, Homo sapiens
caatgtggccgaggactttgattgc

SEQ ID NO: 151 - DNA, 25 bp, Homo sapiens
ggccgaggactttgattgcacattg

SEQ ID NO: 152 - DNA, 25 bp, Homo sapiens
tgacgtggacatccgcaaagacctg

SEQ ID NO: 153 - DNA, 25 bp, Homo sapiens
gtacgccaacacagtgctgtctggc

SEQ ID NO: 154 - DNA, 25 bp, Homo sapiens
caacacagtgctgtctggcggcacc

SEQ ID NO: 155 - DNA, 25 bp, Homo sapiens
gtctggcggcaccaccatgtaccct

SEQ ID NO: 156 - DNA, 25 bp, Homo sapiens
caccatgtaccctggcattgccgac

SEQ ID NO: 157 - DNA, 25 bp, Homo sapiens
gtaccctggcattgccgacaggatg

SEQ ID NO: 158 - DNA, 25 bp, Homo sapiens
tgccgacaggatgcagaaggagatc

SEQ ID NO: 159 - DNA, 25 bp, Homo sapiens
ggagatcactgccctggcacccagc

SEQ ID NO: 160 - DNA, 25 bp, Homo sapiens
cctggcacccagcacaatgaagatc

SEQ ID NO: 161 - DNA, 25 bp, Homo sapiens
acccagcacaatgaagatcaagatc

SEQ ID NO: 162 - DNA, 25 bp, Homo sapiens
tgaagcactacaggaggaatgcacc

SEQ ID NO: 163 - DNA, 25 bp, Homo sapiens
agctctccgccaatttctctcagat

SEQ ID NO: 164 - DNA, 25 bp, Homo sapiens
aatgtacatgggccgcaccataatg

SEQ ID NO: 165 - DNA, 25 bp, Homo sapiens
catgggccgcaccataatgagatgt

SEQ ID NO: 166 - DNA, 25 bp, Homo sapiens
ccgcaccataatgagatgtgagcct

SEQ ID NO: 167 - DNA, 25 bp, Homo sapiens
tggctgttaacccactgcatgcaga

SEQ ID NO: 168 - DNA, 25 bp, Homo sapiens
ttaacccactgcatgcagaaacttg

SEQ ID NO: 169 - DNA, 25 bp, Homo sapiens
cactgcatgcagaaacttggatgtc

SEQ ID NO: 170 - DNA, 25 bp, Homo sapiens
tggaattgactgcctatgccaagtc

SEQ ID NO: 171 - DNA, 25 bp, Homo sapiens
tgactgcctatgccaagtccctgga

SEQ ID NO: 172 - DNA, 25 bp, Homo sapiens
ctcataaaacatgaatcaagcaatc

SEQ ID NO: 173 - DNA, 25 bp, Homo sapiens
gaatcaagcaatccagcctcatggg

SEQ ID NO: 174 - DNA, 25 bp, Homo sapiens
ttgtaaagcccttgcacagctggag

SEQ ID NO: 175 - DNA, 25 bp, Homo sapiens
tgcacagctggagaaatggcatcat

SEQ ID NO: 176 - DNA, 25 bp, Homo sapiens
gcatcattataagctatgagttgaa

SEQ ID NO: 177 - DNA, 25 bp, Homo sapiens
aatgttctgtcaaatgtgtctcaca

SEQ ID NO: 178 - DNA, 25 bp, Homo sapiens
aatgtgtctcacatctacacgtggc

SEQ ID NO: 179 - DNA, 25 bp, Homo sapiens
tctcacatctacacgtggcttggag

SEQ ID NO: 180 - DNA, 25 bp, Homo sapiens
ttccctattgtgacagagccatggt

SEQ ID NO: 181 - DNA, 25 bp, Homo sapiens
attgtgacagagccatggtgtgttt

SEQ ID NO: 182 - DNA, 25 bp, Homo sapiens
ttctccctgcactcatgaaacccca

SEQ ID NO: 183 - DNA, 25 bp, Homo sapiens
tctccctgcactcatgaaaccccaa

SEQ ID NO: 184 - DNA, 25 bp, Homo sapiens
gcactcatgaaaccccaataaatat

SEQ ID NO: 185 - DNA, 25 bp, Homo sapiens
cactcatgaaaccccaataaatatc

SEQ ID NO: 186 - DNA, 25 bp, Homo sapiens
actcatgaaaccccaataaatatcc

SEQ ID NO: 187 - DNA, 25 bp, Homo sapiens
ctcatgaaaccccaataaatatcct

SEQ ID NO: 188 - DNA, 25 bp, Homo sapiens
tcatgaaaccccaataaatatcctc

SEQ ID NO: 189 - DNA, 25 bp, Homo sapiens
catgaaaccccaataaatatcctca

SEQ ID NO: 190 - DNA, 25 bp, Homo sapiens
atgaaaccccaataaatatcctcat

SEQ ID NO: 191 - DNA, 25 bp, Homo sapiens
aaaccccaataaatatcctcattga

SEQ ID NO: 192 - DNA, 25 bp, Homo sapiens
aaccccaataaatatcctcattgac

SEQ ID NO: 193 - DNA, 25 bp, Homo sapiens
ggctgtcctagcagttgtggtcatc

SEQ ID NO: 194 - DNA, 25 bp, Homo sapiens
ctgtcctagcagttgtggtcatcgg

SEQ ID NO: 195 - DNA, 25 bp, Homo sapiens
tgtcctagcagttgtggtcatcgga

SEQ ID NO: 196 - DNA, 25 bp, Homo sapiens
gtcctagcagttgtggtcatcggag

SEQ ID NO: 197 - DNA, 25 bp, Homo sapiens
tcctagcagttgtggtcatcggagc

SEQ ID NO: 198 - DNA, 25 bp, Homo sapiens
ctagcagttgtggtcatcggagctg

SEQ ID NO: 199 - DNA, 25 bp, Homo sapiens
tagcagttgtggtcatcggagctgt

SEQ ID NO: 200 - DNA, 25 bp, Homo sapiens
tttattggcatggagtccgctggaa

SEQ ID NO: 201 - DNA, 25 bp, Homo sapiens
attggcatggagtccgctggaattc

SEQ ID NO: 202 - DNA, 25 bp, Homo sapiens
tccgctggaattcatgagacaacct

SEQ ID NO: 203 - DNA, 25 bp, Homo sapiens
ggaattcatgagacaacctacaatt

SEQ ID NO: 204 - DNA, 25 bp, Homo sapiens
attcatgagacaacctacaattcca

SEQ ID NO: 205 - DNA, 25 bp, Homo sapiens
cacaggaagtgcttctaaagtcaga

SEQ ID NO: 206 - DNA, 25 bp, Homo sapiens
aagtgcttctaaagtcagaacaggt

SEQ ID NO: 207 - DNA, 25 bp, Homo sapiens
tgcttctaaagtcagaacaggttct

SEQ ID NO: 208 - DNA, 25 bp, Homo sapiens
agtcagaacaggttctccaaggatc

SEQ ID NO: 209 - DNA, 25 bp, Homo sapiens
aacaggttctccaaggatcccctcg

SEQ ID NO: 210 - DNA, 25 bp, Homo sapiens
ttctccaaggatcccctcgagacta

SEQ ID NO: 211 - DNA, 25 bp, Homo sapiens
tccaaggatcccctcgagactactc

SEQ ID NO: 212 - DNA, 25 bp, Homo sapiens
gatcccctcgagactactctgttac

SEQ ID NO: 213 - DNA, 25 bp, Homo sapiens
ctcgagactactctgttaccagtca

SEQ ID NO: 214 - DNA, 25 bp, Homo sapiens
gagactactctgttaccagtcatga

SEQ ID NO: 215 - DNA, 25 bp, Homo sapiens
actactctgttaccagtcatgaaac

SEQ ID NO: 216 - DNA, 25 bp, Homo sapiens
actctgttaccagtcatgaaacatt

SEQ ID NO: 217 - DNA, 25 bp, Homo sapiens
ctgttaccagtcatgaaacattaaa

SEQ ID NO: 218 - DNA, 25 bp, Homo sapiens
ttaccagtcatgaaacattaaaacc

SEQ ID NO: 219 - DNA, 25 bp, Homo sapiens
atgaaacattaaaacctacaagcct

SEQ ID NO: 220 - DNA, 25 bp, Homo sapiens
ggtttgcctgaggctgtaactgaga

SEQ ID NO: 221 - DNA, 25 bp, Homo sapiens
cctgaggctgtaactgagagaaaga

SEQ ID NO: 222 - DNA, 25 bp, Homo sapiens
attctggggctgtcttatgaaaata

SEQ ID NO: 223 - DNA, 25 bp, Homo sapiens
atagacattctcacataagcccagt

SEQ ID NO: 224 - DNA, 25 bp, Homo sapiens
acataagcccagttcatcaccattt

SEQ ID NO: 225 - DNA, 25 bp, Homo sapiens
tcacattaggctgttggttcaaact

SEQ ID NO: 226 - DNA, 25 bp, Homo sapiens
gagcacggactgtcagttctctggg

SEQ ID NO: 227 - DNA, 25 bp, Homo sapiens
ggactgtcagttctctgggaagtgg

SEQ ID NO: 228 - DNA, 25 bp, Homo sapiens
gaagtggtcagcgcatcctgcaggg

SEQ ID NO: 229 - DNA, 25 bp, Homo sapiens
gtcagcgcatcctgcagggcttctc

SEQ ID NO: 230 - DNA, 25 bp, Homo sapiens
tttggagaaccagggctcttctcag

SEQ ID NO: 231 - DNA, 25 bp, Homo sapiens
gaaccagggctcttctcaggggctc

SEQ ID NO: 232 - DNA, 25 bp, Homo sapiens
ttctcaggggctctagggactgcca

SEQ ID NO: 233 - DNA, 25 bp, Homo sapiens
ctagggactgccaggctgtttcagc

SEQ ID NO: 234 - DNA, 25 bp, Homo sapiens
tttcagccaggaaggccaaaatcaa

SEQ ID NO: 235 - DNA, 25 bp, Homo sapiens
gggatggtcggatctcacaggctga

SEQ ID NO: 236 - DNA, 25 bp, Homo sapiens
gtcggatctcacaggctgagaactc

SEQ ID NO: 237 - DNA, 25 bp, Homo sapiens
tctcacaggctgagaactcgttcac

SEQ ID NO: 238 - DNA, 25 bp, Homo sapiens
cctccaagcatttcatgaaaaagct

SEQ ID NO: 239 - DNA, 25 bp, Homo sapiens
agcatttcatgaaaaagctgcttct

SEQ ID NO: 240 - DNA, 25 bp, Homo sapiens
caggatctgggcccagtccccatgt

SEQ ID NO: 241 - DNA, 25 bp, Homo sapiens
ggcccagtccccatgtgagagcagc

SEQ ID NO: 242 - DNA, 25 bp, Homo sapiens
cccatgtgagagcagcagaggcggt

SEQ ID NO: 243 - DNA, 25 bp, Homo sapiens
agagcagcagaggcggtcttcaaca

SEQ ID NO: 244 - DNA, 25 bp, Homo sapiens
acacagctacagctttcttgctccc

SEQ ID NO: 245 - DNA, 25 bp, Homo sapiens
caagacaaaccaagtcggaacagca

SEQ ID NO: 246 - DNA, 25 bp, Homo sapiens
caagtcggaacagcagataacaatg

SEQ ID NO: 247 - DNA, 25 bp, Homo sapiens
tgcccaatctccatctgtcaacagg

SEQ ID NO: 248 - DNA, 25 bp, Homo sapiens
tgaggtcccaggaagtggccaaaag

SEQ ID NO: 249 - DNA, 25 bp, Homo sapiens
agctagacagatccccgttcctgac

SEQ ID NO: 250 - DNA, 25 bp, Homo sapiens
gacatcacagcagcctccaacacaa

SEQ ID NO: 251 - DNA, 25 bp, Homo sapiens
caacacaaggctccaagacctaggc

SEQ ID NO: 252 - DNA, 25 bp, Homo sapiens
aagacctaggctcatggacgagatg

SEQ ID NO: 253 - DNA, 25 bp, Homo sapiens
ccagaccccaggctggacatgctga

SEQ ID NO: 254 - DNA, 25 bp, Homo sapiens
cctttggccttggcttttctagcct

SEQ ID NO: 255 - DNA, 25 bp, Homo sapiens
ttggcttttctagcctatttacctg

SEQ ID NO: 256 - DNA, 25 bp, Homo sapiens
agcctatttacctgcaggctgagcc

SEQ ID NO: 257 - DNA, 25 bp, Homo sapiens
gctcagccaagcttgttatcagctt

SEQ ID NO: 258 - DNA, 25 bp, Homo sapiens
aagcttgttatcagctttcagggcc

SEQ ID NO: 259 - DNA, 25 bp, Homo sapiens
atcagctttcagggccatggttcac

SEQ ID NO: 260 - DNA, 25 bp, Homo sapiens
tccctgcactcatgaaaccccaata

SEQ ID NO: 261 - DNA, 25 bp, Homo sapiens
ccctgcactcatgaaaccccaataa

SEQ ID NO: 262 - DNA, 25 bp, Homo sapiens
cctgcactcatgaaaccccaataaa

SEQ ID NO: 263 - DNA, 25 bp, Homo sapiens
ctgcactcatgaaaccccaataaat

SEQ ID NO: 264 - DNA, 25 bp, Homo sapiens
tgcactcatgaaaccccaataaata

SEQ ID NO: 265 - DNA, 1793 bp, Homo sapiens
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 266 - DNA, 3528 bp, Homo sapiens
ctttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgtgttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccagtttgatggtttcacaagttctattgagtgctattcagaataggaacaaggttctaatagaaaaagatggcaatttgaagtagctataaaattagactaatctacattgcttttctcctgcagagtctaataccttttatgctttgataattagcagtttgtctacttggtcactaggaatgaaactacatggtaataggcttaacaggtgtaatagcccacttactcctgaatctttaagcatttgtgcatttgaaaaatgcttttcgcgatcttcctgctgggattacaggcatgagccactgtgcctgacctcccatatgtaaaagtgtctaaaggtttttttttggttataaaaggaaaatttttgcttaagtttgaaggataggtaaaattaaaggacatgctttctgtttgtgtgatggtttttaaaaattttttttaagatggagttcttgttgcccaggctagaatgcaatggcaaaatctcactgcaatctcctcctcctgggttcaagcaattctcctacttcagcctcccaagtagctgggattacaggcatgtgctaatttggtgtttttaatagagatgaggtttttccatgttggtcaggctggtctcaaactcctgaccttaggtgatcgcctcggcctcctaaagtgctggaattacaggcatgagccaccatgcctggccaggacatgtgttcttaaggacatgctaagcaggagttaaagcagcccaagagataaggcctcttaaagtgactggcaatgtgtattgctcaagattcaaaggtacttgaattggccatagacaagtctgtaatgaagtgttatcgttttccctcatctgagtctgaattagataaaatgccttcccatcagccagtgctctgaggtatcaagtctaaattgaactagagatttttgtccttagtttctttgctatctaatgtttacacaagtaaatagtctaagatttgctggatgacagaaaaaacaggtaaggcctttaatagatggccaatagatgccctgataatgaaagttgacacctgtaagatttaccagtagagaattcttgacatgcaaggaagcaagatttaactgaaaaattgttcccactggaagcaggaatgagtcagtttacttgcatatactgagattgagattaacttcctgtgaaacccagtgtcttagacaactgtggcttgagcaccacctgctggtattcattacaaacttgctcactacaataaatgaattttaagctttaaaaaaaaaaaaaaaaaa

SEQ ID NO: 267 - DNA, 2527 bp, Homo sapiens
ctcaagctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccattcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatggccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtcgctctgatatagcagccctggtgtattttcgatatttcaggaagactggcagattggaccagaccctgaattcttctagctcctccaatcccattttatccatggaaccactaaaaacaaggtctgctctgctcctgaagccctatatgctggagatggacaactcaatgaaaatttaaagggaaaaccctcaggcctgaggtgtgtgccactcagagacttcacctaactagagacaggcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtattcacaagaagtagcttcagagggtaacttaacagagtatcagattctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaatttcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgctggtaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgcttaagatttggtcacactctctcacctaggtgagcgcattgagccagtggtgctaaatgctacatactccaactgaaatgttaaggaagaagatagatccaattaaaaaaaattaaaaccaatttaaaaaaaaaaagaacacaggagattccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgccaggggggagctatcactgtacttgtagagtggtgctgctttaattcataaatcacaaataaaagccaattagctctataact

SEQ ID NO: 268 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 269 - DNA, 2691 bp, Homo sapiens
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 270 - DNA, 914 bp, Homo sapiens
tgtggcagatttcagaggcccttaaaatgaggccaagtgaggtggacaggtccgagccagctgaggactcctcagccacacggcacagctgcctgaggggatgtgtcactcagggagttgctgggacctactgggcccagcgttgccatcagcaccaacagcttcagagagggggacacatgccggggtgactccaaggctgtgggcggcacctgcctcagatagagaacaggcacagagacactactgggggacactactgggacactggccacccccctaccctgtgcctagatcacagcctacacactgcagccctgtgcccctcacacccagcaggttcctgctccagcgcggctcctggactggccccgggtgctggccccgggggtttcaatccaagcataattcagtgaagcatgtgtttggcagcgggacccagctcaccgttttaggtcagcccaaggccaccccctcggtcactctgttcctgccgtcctctgaggagctccaagccaacaaggccacactggtgtgtctcatgaatgacttctatctgggaatcttgacggtgacctggaaggcagatggtacccccatcacccagggcgtggagatgaccacgccctccaaacagagcaacagcaagtacatggccagcagctacctgagcctgacgcccgagcagtggaggtcccgcagaagctacagctgccaggtcatgcatgaagggagcactgcagagaagacggtggcccctgcagaatgttcataggttcccagcccccaccccacccacaggggcctggagctgcaggatcccaggggaggcgtctctctctgcatcccaagccatccagcccttctccctgtacccagtaaaccctcagtaaatatcctctttgtcaaccaga

SEQ ID NO: 271 - DNA, 1533 bp, Homo sapiens
atgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttacgtgcactgagctgcaacttcttacttccctactgaaaataagaatctgaatataaatttgttttctcaaatatttgctatgagaggttgatggattaattaaataagtcaattcctggaatttgaaagagcaaataaagacctgagaaccttccagaaaaaaaaaaaaaaaaa

SEQ ID NO: 272 - DNA, 1345 bp, Homo sapiens
gcctctggggttttatattgctctggtattcatgccaaagacacaccagccctcagtcactgggagaagaacctctcataccctcggtgctccagtccccagctcactcagccacacacaccatgtgtgaagaggagaccaccgcgctcgtgtgtgacaatggctctggcctgtgcaaggcaggcttcgcaggagatgatgccccccgggctgtcttcccctccattgtgggccgccctcgccaccagggtgtgatggtgggaatgggccagaaagacagctatgtgggggatgaggctcagagcaagcgagggatcctaactctcaaataccccattgaacacggcatcatcaccaactgggatgacatggagaagatctggcaccactccttctacaatgagctgcgtgtagcacctgaagagcaccccaccctgctcacagaggctcccctaaatcccaaggccaacagggaaaagatgacccagatcatgtttgaaaccttcaatgtccctgccatgtacgtcgccattcaagctgtgctctccctctatgcctctggccgcacgacaggcatcgtcctggattcaggtgatggcgtcacccacaatgtccccatctatgaaggctatgccctgccccatgccatcatgcgcctggacttggctggccgtgacctcacggactacctcatgaagatcctcacagagagaggctattcctttgtgaccacagctgagagagaaattgtgcgagacatcaaggagaagctgtgctatgtggccctggattttgagaatgagatggccacagcagcttcctcttcctccctggagaagagctatgagctgccagatgggcaggttatcaccattggcaatgagcgcttccgctgccctgagaccctcttccagccttcctttattggcatggagtccgctggaattcatgagacaacctacaattccatcatgaagtgtgacattgacatccgtaaggacttatatgccaacaatgtcctctctgggggcaccaccatgtaccctggcattgctgacaggatgcagaaggagatcacagccctggcccccagcaccatgaagatcaagattattgctcccccagagcggaagtactcagtctggatcgggggctctatcctggcctctctctccaccttccagcagatgtggatcagcaagcctgagtatgatgaggcagggccctccattgtccacaggaagtgcttctaaagtcagaacaggttctccaaggatcccctcgagactactctgttaccagtcatgaaacattaaaacctacaagccttaaaaaaaaaaaaaaa

SEQ ID NO: 273 - DNA, 8815 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 274 - DNA, 3178 bp, Homo sapiens
gttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaaaaaaaaaaaaaaaaaaaaaaaaaaaaaaaaaaaaaaa

SEQ ID NO: 275 - DNA, 1519 bp, Homo sapiens
cagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaaaaaaaaaaaaaaaaaa

SEQ ID NO: 276 - DNA, 901 bp, Homo sapiens
ggccacatggactggggtgcaatgggacagctgctgccagcgagagggaccagggcaccactctctagggagcccacactgcaagtcaggccacaaggacctctgaccctgagggccgatgaggccagggacaggccaggggggccttgaggcccctggtgagccaggccccaacctcaggcagcgctggcccctgctgctgctgggtctggccgtggtaacccatggcctgctgcgcccaacagctgcatcgcagagcagggccctgggccctggagcccctggaggaagcagccggtccagcctgaggagccggtggggcaggttcctgctccagcgcggctcctggactggccccaggtgctggccccgggggtttcaatccaagcataactcagtgacgcatgtgtttggcagcgggacccagctcaccgttttaagtcagcccaaggccaccccctcggtcactctgttcccgccgtcctctgaggagctccaagccaacaaggctacactggtgtgtctcatgaatgacttttatccgggaatcttgacggtgacctggaaggcagatggtacccccatcacccagggcgtggagatgaccacgccctccaaacagagcaacaacaagtacgcggccagcagctacctgagcctgacgcccgagcagtggaggtcccgcagaagctacagctgccaggtcatgcacgaagggagcaccgtggagaagacggtggcccctgcagaatgttcataggttcccagccccgaccccacccaaaggggcctggagctgcaggatcccaggggaagggtctctctctgcatcccaagccatccagcccttctccctgtacccagtaaaccctaaataaataccctctttgtcaaccagaaa

SEQ ID NO: 277 - DNA, 23 bp, Homo sapiens
ctgactgactgactgactgactg

SEQ ID NO: 278 - DNA, 21 bp, Homo sapiens
ttgtacgtttacatggaggtc

SEQ ID NO: 279 - DNA, 22 bp, Homo sapiens
aaccacacaacctactacctca

SEQ ID NO: 280 - DNA, 20 bp, Homo sapiens
tgtattcctcgcctgtccag

SEQ ID NO: 281 - DNA, 22 bp, Homo sapiens
tcgccctctcaacccagctttt

SEQ ID NO: 282 - DNA, 22 bp, Homo sapiens
aactatacaacctactacctca

SEQ ID NO: 283 - DNA, 22 bp, Homo sapiens
aaccatacaacctactacctca

SEQ ID NO: 284 - DNA, 21 bp, Homo sapiens
gctgagtgtaggatgtttaca

SEQ ID NO: 285 - DNA, 22 bp, Homo sapiens
acacaaattcggttctacaggg

SEQ ID NO: 286 - DNA, 20 bp, Homo sapiens
cattgaggctcgctgagagt

SEQ ID NO: 287 - DNA, 21 bp, Homo sapiens
tgagctcctggaggacaggga

SEQ ID NO: 288 - DNA, 20 bp, Homo sapiens
cttgtaccagttatctgcaa

SEQ ID NO: 289 - RNA, 68 nt, Homo sapiens
ccucuacuuuaacauggaggcacuugcugugacaugacaaaaauaagugcuuccauguuugagugugg

SEQ ID NO: 290 - RNA, 83 nt, Homo sapiens
cggggugagguaguagguugugugguuucagggcagugauguugccccucggaagauaacuauacaaccuacugccuucccug

SEQ ID NO: 291 - RNA, 83 nt, Homo sapiens
gugagugggagccccagugugugguuggggccauggcgggugggcagcccagccucugagccuuccucgucugucugccccag

SEQ ID NO: 292 - RNA, 82 nt, Homo sapiens
gcuucgcuccccuccgccuucucuucccgguucuucccggagucgggaaaagcuggguugagagggcgaaaaaggaugaggu

SEQ ID NO: 293 - RNA, 80 nt, Homo sapiens
ugggaugagguaguagguuguauaguuuuagggucacacccaccacugggagauaacuauacaaucuacugucuuuccua

SEQ ID NO: 294 - RNA, 84 nt, Homo sapiens
gcauccggguugagguaguagguuguaugguuuagaguuacacccugggaguuaacuguacaaccuucuagcuuuccuuggagc

SEQ ID NO: 295 - RNA, 88 nt, Homo sapiens
accaaguuucaguucauguaaacauccuacacucagcuguaauacauggauuggcugggagguggauguuuacuucagcugacuugga

SEQ ID NO: 296 - RNA, 110 nt, Homo sapiens; identical to SEQ ID NO: 36

SEQ ID NO: 297 - RNA, 84 nt, Homo sapiens
cuccccauggcccugucucccaacccuuguaccagugcugggcucagacccugguacaggccugggggacagggaccuggggac

SEQ ID NO: 298 - DNA, 25 bp, Homo sapiens
ggaaagcgccgagatgacgggcttt

SEQ ID NO: 299 - DNA, 25 bp, Homo sapiens
gatgacgggctttctgctgccgccc

SEQ ID NO: 300 - DNA, 25 bp, Homo sapiens
cccaagtagctttgtggcttcgtgt

SEQ ID NO: 301 - DNA, 25 bp, Homo sapiens
tagctttgtggcttcgtgtccaacc

SEQ ID NO: 302 - DNA, 25 bp, Homo sapiens
tgtggcttcgtgtccaaccctcttg

SEQ ID NO: 303 - DNA, 25 bp, Homo sapiens
cgcctgtgtgcctggagccagtccc

SEQ ID NO: 304 - DNA, 25 bp, Homo sapiens
gctcgcgtttcctcctgtagtgctc

SEQ ID NO: 305 - DNA, 25 bp, Homo sapiens
gtttcctcctgtagtgctcacaggt

SEQ ID NO: 306 - DNA, 25 bp, Homo sapiens
agtgctcacaggtcccagcaccgat

SEQ ID NO: 307 - DNA, 25 bp, Homo sapiens
tcccagcaccgatggcattcccttt

SEQ ID NO: 308 - DNA, 25 bp, Homo sapiens
tccctttgccctgagtctgcagcgg

SEQ ID NO: 309 - DNA, 25 bp, Homo sapiens
tgccctgagtctgcagcgggtccct

SEQ ID NO: 310 - DNA, 25 bp, Homo sapiens
tcaggtagcctctcttccccttggg

SEQ ID NO: 311 - DNA, 25 bp, Homo sapiens
acccgcggtaaccagcgtgagctcg

SEQ ID NO: 312 - DNA, 25 bp, Homo sapiens
gcccgccagaagaatatgaaaaagc

SEQ ID NO: 313 - DNA, 25 bp, Homo sapiens
gactcggttaagggaaagcgccgag

SEQ ID NO: 314 - DNA, 25 bp, Homo sapiens
ttatgaatgtccaaatctgtgtttc

SEQ ID NO: 315 - DNA, 25 bp, Homo sapiens
atgaatgtccaaatctgtgtttccc

SEQ ID NO: 316 - DNA, 25 bp, Homo sapiens
gaatgtccaaatctgtgtttccccc

SEQ ID NO: 317 - DNA, 25 bp, Homo sapiens
atgtccaaatctgtgtttccccctg

SEQ ID NO: 318 - DNA, 25 bp, Homo sapiens
ctcccagactgtgtggccagttgaa

SEQ ID NO: 319 - DNA, 25 bp, Homo sapiens
agactgtgtggccagttgaaagtgt

SEQ ID NO: 320 - DNA, 25 bp, Homo sapiens
actgtgtggccagttgaaagtgtct

SEQ ID NO: 321 - DNA, 25 bp, Homo sapiens
tggccagttgaaagtgtctggtttg

SEQ ID NO: 322 - DNA, 25 bp, Homo sapiens
ttgaaagtgtctggtttgtgttcat

SEQ ID NO: 323 - DNA, 25 bp, Homo sapiens
agtgtctggtttgtgttcatctctc

SEQ ID NO: 324 - DNA, 25 bp, Homo sapiens
tgtctggtttgtgttcatctctccc

SEQ ID NO: 325 - DNA, 25 bp, Homo sapiens
gtgttcatctctccctcatttctgg

SEQ ID NO: 326 - DNA, 25 bp, Homo sapiens
tgcatccacgcctcttttggacatt

SEQ ID NO: 327 - DNA, 25 bp, Homo sapiens
catccacgcctcttttggacattaa

SEQ ID NO: 328 - DNA, 25 bp, Homo sapiens
tccacgcctcttttggacattaaag

SEQ ID NO: 329 - DNA, 25 bp, Homo sapiens
ggtggccttcttgcaggtccccgta

SEQ ID NO: 330 - DNA, 25 bp, Homo sapiens
tggccttcttgcaggtccccgtagc

SEQ ID NO: 331 - DNA, 25 bp, Homo sapiens
ggccttcttgcaggtccccgtagca

SEQ ID NO: 332 - DNA, 25 bp, Homo sapiens
gccttcttgcaggtccccgtagcac

SEQ ID NO: 333 - DNA, 25 bp, Homo sapiens
tcttgcaggtccccgtagcaccctg

SEQ ID NO: 334 - DNA, 25 bp, Homo sapiens
tgcaggtccccgtagcaccctgagc

SEQ ID NO: 335 - DNA, 25 bp, Homo sapiens
aggtccccgtagcaccctgagcctg

SEQ ID NO: 336 - DNA, 25 bp, Homo sapiens
ggtccccgtagcaccctgagcctgt

SEQ ID NO: 337 - DNA, 25 bp, Homo sapiens
ccgtagcaccctgagcctgtacctt

SEQ ID NO: 338 - DNA, 25 bp, Homo sapiens
tagcaccctgagcctgtaccttggg

SEQ ID NO: 339 - DNA, 25 bp, Homo sapiens
caccctgagcctgtaccttgggtgg

SEQ ID NO: 340 - DNA, 25 bp, Homo sapiens
accctgagcctgtaccttgggtggc

SEQ ID NO: 341 - DNA, 25 bp, Homo sapiens
ccctgagcctgtaccttgggtggca

SEQ ID NO: 342 - DNA, 25 bp, Homo sapiens
gagcctgtaccttgggtggcacttg

SEQ ID NO: 343 - DNA, 25 bp, Homo sapiens
gcctgtaccttgggtggcacttgtt

SEQ ID NO: 344 - DNA, 25 bp, Homo sapiens
tgctgcctctggggacatgcggagt

SEQ ID NO: 345 - DNA, 25 bp, Homo sapiens
ggggaagccttcctctcaatttgtt

SEQ ID NO: 346 - DNA, 25 bp, Homo sapiens
gggaagccttcctctcaatttgttg

SEQ ID NO: 347 - DNA, 25 bp, Homo sapiens
ggaagccttcctctcaatttgttgt

SEQ ID NO: 348 - DNA, 25 bp, Homo sapiens
gaagccttcctctcaatttgttgtc

SEQ ID NO: 349 - DNA, 25 bp, Homo sapiens
aagccttcctctcaatttgttgtca

SEQ ID NO: 350 - DNA, 25 bp, Homo sapiens
agccttcctctcaatttgttgtcag

SEQ ID NO: 351 - DNA, 25 bp, Homo sapiens
ccttcctctcaatttgttgtcagtg

SEQ ID NO: 352 - DNA, 25 bp, Homo sapiens
cttcctctcaatttgttgtcagtga

SEQ ID NO: 353 - DNA, 25 bp, Homo sapiens
ttcctctcaatttgttgtcagtgaa

SEQ ID NO: 354 - DNA, 25 bp, Homo sapiens
tcctctcaatttgttgtcagtgaaa

SEQ ID NO: 355 - DNA, 25 bp, Homo sapiens
cctctcaatttgttgtcagtgaaat

SEQ ID NO: 356 - DNA, 25 bp, Homo sapiens
ctctcaatttgttgtcagtgaaatt

SEQ ID NO: 357 - DNA, 25 bp, Homo sapiens
aattccaataaatgggatttgctct

SEQ ID NO: 358 - DNA, 25 bp, Homo sapiens
tgagggtgcacgtcttccctcctgt

SEQ ID NO: 359 - DNA, 25 bp, Homo sapiens
tggagtgctgcctctggggacatgc

SEQ ID NO: 360 - DNA, 25 bp, Homo sapiens
ggttaatccgcaagccccagccccg

SEQ ID NO: 361 - DNA, 25 bp, Homo sapiens
ttaatccgcaagccccagccccgag

SEQ ID NO: 362 - DNA, 25 bp, Homo sapiens
ggcgtcccccagagcctgagaaagc

SEQ ID NO: 363 - DNA, 25 bp, Homo sapiens
ccccagagcctgagaaagcgcctcc

SEQ ID NO: 364 - DNA, 25 bp, Homo sapiens
ccagagcctgagaaagcgcctcccg

SEQ ID NO: 365 - DNA, 25 bp, Homo sapiens
gagcctgagaaagcgcctcccgctg

SEQ ID NO: 366 - DNA, 25 bp, Homo sapiens
gcctgagaaagcgcctcccgctgcc

SEQ ID NO: 367 - DNA, 25 bp, Homo sapiens
ctgagaaagcgcctcccgctgcccc

SEQ ID NO: 368 - DNA, 25 bp, Homo sapiens
tgccccgacgcggccctcggccctg

SEQ ID NO: 369 - DNA, 25 bp, Homo sapiens
ctcggccctggagctgaaggtggag

SEQ ID NO: 370 - DNA, 25 bp, Homo sapiens
cggccctggagctgaaggtggagga

SEQ ID NO: 371 - DNA, 25 bp, Homo sapiens
gccctggagctgaaggtggaggagc

SEQ ID NO: 372 - DNA, 25 bp, Homo sapiens
cctggagctgaaggtggaggagctg

SEQ ID NO: 373 - DNA, 25 bp, Homo sapiens
gctgaaggtggaggagctggaggag

SEQ ID NO: 374 - DNA, 25 bp, Homo sapiens
aaggtggaggagctggaggagaagg

SEQ ID NO: 375 - DNA, 25 bp, Homo sapiens
gactgcttgaaaccaggagtttgag

SEQ ID NO: 376 - DNA, 25 bp, Homo sapiens
gcttgaaaccaggagtttgagacca

SEQ ID NO: 377 - DNA, 25 bp, Homo sapiens
aaccaggagtttgagaccagcctga

SEQ ID NO: 378 - DNA, 25 bp, Homo sapiens
ttgagaccagcctgagcaacaaagc

SEQ ID NO: 379 - DNA, 25 bp, Homo sapiens
agaccagcctgagcaacaaagcaag

SEQ ID NO: 380 - DNA, 25 bp, Homo sapiens
gagcaacaaagcaagaccccatctc

SEQ ID NO: 381 - DNA, 25 bp, Homo sapiens
caacaaagcaagaccccatctctat

SEQ ID NO: 382 - DNA, 25 bp, Homo sapiens
aagcaagaccccatctctataaaaa

SEQ ID NO: 383 - DNA, 25 bp, Homo sapiens
aagacagggtcttgctcatgttgta

SEQ ID NO: 384 - DNA, 25 bp, Homo sapiens
attagttgggcatggtggcacatgc

SEQ ID NO: 385 - DNA, 25 bp, Homo sapiens
agttgggcatggtggcacatgcctg

SEQ ID NO: 386 - DNA, 25 bp, Homo sapiens
atcatctgagcctcaggaggttgag

SEQ ID NO: 387 - DNA, 25 bp, Homo sapiens
atctgagcctcaggaggttgaggct

SEQ ID NO: 388 - DNA, 25 bp, Homo sapiens
tgaggctgcagtgagctgtgactgc

SEQ ID NO: 389 - DNA, 25 bp, Homo sapiens
cttgctcatgttgtacattcatcat

SEQ ID NO: 390 - DNA, 25 bp, Homo sapiens
aagaggctgggtgcagtggctcaca

SEQ ID NO: 391 - DNA, 25 bp, Homo sapiens
tcatttcctggtatgacaacgaatt

SEQ ID NO: 392 - DNA, 25 bp, Homo sapiens
acaacgaatttggctacagcaacag

SEQ ID NO: 393 - DNA, 25 bp, Homo sapiens
gggtggtggacctcatggcccacat

SEQ ID NO: 394 - DNA, 25 bp, Homo sapiens
tcatggcccacatggcctccaagga

SEQ ID NO: 395 - DNA, 25 bp, Homo sapiens
acatggcctccaaggagtaagaccc

SEQ ID NO: 396 - DNA, 25 bp, Homo sapiens
aggagtaagacccctggaccaccag

SEQ ID NO: 397 - DNA, 25 bp, Homo sapiens
gccccagcaagagcacaagaggaag

SEQ ID NO: 398 - DNA, 25 bp, Homo sapiens
gagagagaccctcactgctggggag

SEQ ID NO: 399 - DNA, 25 bp, Homo sapiens
cctcactgctggggagtccctgcca

SEQ ID NO: 400 - DNA, 25 bp, Homo sapiens
cctcctcacagttgccatgtagacc

SEQ ID NO: 401 - DNA, 25 bp, Homo sapiens
agttgccatgtagaccccttgaaga

SEQ ID NO: 402 - DNA, 25 bp, Homo sapiens
catgtagaccccttgaagaggggag

SEQ ID NO: 403 - DNA, 25 bp, Homo sapiens
tagggagccgcaccttgtcatgtac

SEQ ID NO: 404 - DNA, 25 bp, Homo sapiens
gccgcaccttgtcatgtaccatcaa

SEQ ID NO: 405 - DNA, 25 bp, Homo sapiens
tgtcatgtaccatcaataaagtacc

SEQ ID NO: 406 - DNA, 25 bp, Homo sapiens
cctctgacttcaacagcgacaccca

SEQ ID NO: 407 - DNA, 25 bp, Homo sapiens
gggctggcattgccctcaacgacca

SEQ ID NO: 408 - DNA, 25 bp, Homo sapiens
ccctcaacgaccactttgtcaagct

SEQ ID NO: 409 - DNA, 25 bp, Homo sapiens
accactttgtcaagctcatttcctg

SEQ ID NO: 410 - DNA, 25 bp, Homo sapiens
ttgtcaagctcatttcctggtatga

SEQ ID NO: 411 - DNA, 25 bp, Homo sapiens
gaattctggtaccgtcagcatccac

SEQ ID NO: 412 - DNA, 25 bp, Homo sapiens
gagagagacctcatctttcatgctt

SEQ ID NO: 413 - DNA, 25 bp, Homo sapiens
tgactctcctgggggcacctcctat

SEQ ID NO: 414 - DNA, 25 bp, Homo sapiens
actctcctgggggcacctcctatga

SEQ ID NO: 415 - DNA, 25 bp, Homo sapiens
tcctgggggcacctcctatgagaga

SEQ ID NO: 416 - DNA, 25 bp, Homo sapiens
cctgggggcacctcctatgagagat

SEQ ID NO: 417 - DNA, 25 bp, Homo sapiens
ctgggggcacctcctatgagagata

SEQ ID NO: 418 - DNA, 25 bp, Homo sapiens
tgggggcacctcctatgagagatac

SEQ ID NO: 419 - DNA, 25 bp, Homo sapiens
gggggcacctcctatgagagatacg

SEQ ID NO: 420 - DNA, 25 bp, Homo sapiens
ggggcacctcctatgagagatacga

SEQ ID NO: 421 - DNA, 25 bp, Homo sapiens
gggcacctcctatgagagatacgat

SEQ ID NO: 422 - DNA, 25 bp, Homo sapiens
ggcacctcctatgagagatacgatt

SEQ ID NO: 423 - DNA, 25 bp, Homo sapiens
gcacctcctatgagagatacgattg

SEQ ID NO: 424 - DNA, 25 bp, Homo sapiens
cacctcctatgagagatacgattgc

SEQ ID NO: 425 - DNA, 25 bp, Homo sapiens
acctcctatgagagatacgattgct

SEQ ID NO: 426 - DNA, 25 bp, Homo sapiens
cctcctatgagagatacgattgcta

SEQ ID NO: 427 - DNA, 25 bp, Homo sapiens
ctcctattccggactcagacctctg

SEQ ID NO: 428 - DNA, 25 bp, Homo sapiens
tcctattccggactcagacctctga

SEQ ID NO: 429 - DNA, 25 bp, Homo sapiens
cctattccggactcagacctctgac

SEQ ID NO: 430 - DNA, 25 bp, Homo sapiens
ctattccggactcagacctctgacc

SEQ ID NO: 431 - DNA, 25 bp, Homo sapiens
attccggactcagacctctgaccct

SEQ ID NO: 432 - DNA, 25 bp, Homo sapiens
ttccggactcagacctctgaccctg

SEQ ID NO: 433 - DNA, 25 bp, Homo sapiens
cggactcagacctctgaccctgcaa

SEQ ID NO: 434 - DNA, 25 bp, Homo sapiens
ggactcagacctctgaccctgcaat

SEQ ID NO: 435 - DNA, 25 bp, Homo sapiens
actcagacctctgaccctgcaatgc

SEQ ID NO: 436 - DNA, 25 bp, Homo sapiens
cagacctctgaccctgcaatgctgc

SEQ ID NO: 437 - DNA, 25 bp, Homo sapiens
acctctgaccctgcaatgctgccta

SEQ ID NO: 438 - DNA, 25 bp, Homo sapiens
tctgaccctgcaatgctgcctacca

SEQ ID NO: 439 - DNA, 25 bp, Homo sapiens
ctgaccctgcaatgctgcctaccat

SEQ ID NO: 440 - DNA, 25 bp, Homo sapiens
tgaccctgcaatgctgcctaccatg

SEQ ID NO: 441 - DNA, 25 bp, Homo sapiens
accctgcaatgctgcctaccatgat

SEQ ID NO: 442 - DNA, 25 bp, Homo sapiens
cctgcaatgctgcctaccatgattg

SEQ ID NO: 443 - DNA, 25 bp, Homo sapiens
aggcacgtaccaccatgcccagata

SEQ ID NO: 444 - DNA, 25 bp, Homo sapiens
ttttttgagacaaagtcctcactct

SEQ ID NO: 445 - DNA, 25 bp, Homo sapiens
ggggtttcaccatgttggctaggat

SEQ ID NO: 446 - DNA, 25 bp, Homo sapiens
ccatgttggctaggatggtctccat

SEQ ID NO: 447 - DNA, 25 bp, Homo sapiens
gttggctaggatggtctccatcgcc

SEQ ID NO: 448 - DNA, 25 bp, Homo sapiens
ctaggatggtctccatcgcctgacc

SEQ ID NO: 449 - DNA, 25 bp, Homo sapiens
tgagacaaagtcctcactctgtcac

SEQ ID NO: 450 - DNA, 25 bp, Homo sapiens
cttggcctcccaaagtgctgggatt

SEQ ID NO: 451 - DNA, 25 bp, Homo sapiens
cctcccaaagtgctgggattacagg

SEQ ID NO: 452 - DNA, 25 bp, Homo sapiens
ggattacaggcatgagccaccacag

SEQ ID NO: 453 - DNA, 25 bp, Homo sapiens
caaagtcctcactctgtcaccaagt

SEQ ID NO: 454 - DNA, 25 bp, Homo sapiens
gcatgagccaccacagctggccgta

SEQ ID NO: 455 - DNA, 25 bp, Homo sapiens
gagccaccacagctggccgtaaata

SEQ ID NO: 456 - DNA, 25 bp, Homo sapiens
gtgcagtggcagcaatctcagctca

SEQ ID NO: 457 - DNA, 25 bp, Homo sapiens
gtggcagcaatctcagctcactgca

SEQ ID NO: 458 - DNA, 25 bp, Homo sapiens
agcaatctcagctcactgcaaacct

SEQ ID NO: 459 - DNA, 25 bp, Homo sapiens
caccgcgcctggccctaaatagatt

SEQ ID NO: 460 - DNA, 25 bp, Homo sapiens
gggattcatcatgttgaccaggctg

SEQ ID NO: 461 - DNA, 25 bp, Homo sapiens
ttcatcatgttgaccaggctggcct

SEQ ID NO: 462 - DNA, 25 bp, Homo sapiens
tgtttgtctttctgataggttgaaa

SEQ ID NO: 463 - DNA, 25 bp, Homo sapiens
tgtctttctgataggttgaaaattg

SEQ ID NO: 464 - DNA, 25 bp, Homo sapiens
gttgaccaggctggcctcaaactcc

SEQ ID NO: 465 - DNA, 25 bp, Homo sapiens
accaggctggcctcaaactcctgac

SEQ ID NO: 466 - DNA, 25 bp, Homo sapiens
aggctggcctcaaactcctgacttc

SEQ ID NO: 467 - DNA, 25 bp, Homo sapiens
tggcctcaaactcctgacttcaagc

SEQ ID NO: 468 - DNA, 25 bp, Homo sapiens
ctcaaactcctgacttcaagcgatc

SEQ ID NO: 469 - DNA, 25 bp, Homo sapiens
aaactcctgacttcaagcgatctcc

SEQ ID NO: 470 - DNA, 25 bp, Homo sapiens
ttggcctcccaaagtgctgggattg

SEQ ID NO: 471 - DNA, 25 bp, Homo sapiens
cctcccaaagtgctgggattgcagg

SEQ ID NO: 472 - DNA, 25 bp, Homo sapiens
gctgggattgcaggtgtgagccacc

SEQ ID NO: 473 - DNA, 25 bp, Homo sapiens
attgcaggtgtgagccaccgcgcct

SEQ ID NO: 474 - DNA, 25 bp, Homo sapiens
tcttgacaaaacctaacttgcgcag

SEQ ID NO: 475 - DNA, 25 bp, Homo sapiens
atgagattggcatggctttatttgt

SEQ ID NO: 476 - DNA, 25 bp, Homo sapiens
gcagtcggttggagcgagcatcccc

SEQ ID NO: 477 - DNA, 25 bp, Homo sapiens
ccaaagttcacaatgtggccgagga

SEQ ID NO: 478 - DNA, 25 bp, Homo sapiens
aagttcacaatgtggccgaggactt

SEQ ID NO: 479 - DNA, 25 bp, Homo sapiens
atgtggccgaggactttgattgcac

SEQ ID NO: 480 - DNA, 25 bp, Homo sapiens
ccgaggactttgattgcacattgtt

SEQ ID NO: 481 - DNA, 25 bp, Homo sapiens
tttaatagtcattccaaatatgaga

SEQ ID NO: 482 - DNA, 25 bp, Homo sapiens
agtcattccaaatatgagatgcatt

SEQ ID NO: 483 - DNA, 25 bp, Homo sapiens
tgttacaggaagtcccttgccatcc

SEQ ID NO: 484 - DNA, 25 bp, Homo sapiens
tacaggaagtcccttgccatcctaa

SEQ ID NO: 485 - DNA, 25 bp, Homo sapiens
tcccttgccatcctaaaagccaccc

SEQ ID NO: 486 - DNA, 25 bp, Homo sapiens
cttctctctaaggagaatggcccag

SEQ ID NO: 487 - DNA, 25 bp, Homo sapiens
gaggtgatagcattgctttcgtgta

SEQ ID NO: 488 - DNA, 25 bp, Homo sapiens
tattttgaatgatgagccttcgtgc

SEQ ID NO: 489 - DNA, 25 bp, Homo sapiens
tttgaatgatgagccttcgtgcccc

SEQ ID NO: 490 - DNA, 25 bp, Homo sapiens
gtatgaaggcttttggtctccctgg

SEQ ID NO: 491 - DNA, 25 bp, Homo sapiens
ggtggaggcagccagggcttacctg

SEQ ID NO: 492 - DNA, 25 bp, Homo sapiens
cagggcttacctgtacactgacttg

SEQ ID NO: 493 - DNA, 25 bp, Homo sapiens
ttacctgtacactgacttgagacca

SEQ ID NO: 494 - DNA, 25 bp, Homo sapiens
gcctggccaacatggtgaaaccccg

SEQ ID NO: 495 - DNA, 25 bp, Homo sapiens
gcgcgcgcctgtaatcccagctact

SEQ ID NO: 496 - DNA, 25 bp, Homo sapiens
gcgcgcctgtaatcccagctactcg

SEQ ID NO: 497 - DNA, 25 bp, Homo sapiens
cgcgcctgtaatcccagctactcgg

SEQ ID NO: 498 - DNA, 25 bp, Homo sapiens
gcgcctgtaatcccagctactcggg

SEQ ID NO: 499 - DNA, 25 bp, Homo sapiens
cgcctgtaatcccagctactcggga

SEQ ID NO: 500 - DNA, 25 bp, Homo sapiens
gcctgtaatcccagctactcgggag

SEQ ID NO: 501 - DNA, 25 bp, Homo sapiens
cctgtaatcccagctactcgggagg

SEQ ID NO: 502 - DNA, 25 bp, Homo sapiens
ctgtaatcccagctactcgggaggc

SEQ ID NO: 503 - DNA, 25 bp, Homo sapiens
tgtaatcccagctactcgggaggct

SEQ ID NO: 504 - DNA, 25 bp, Homo sapiens
gtaatcccagctactcgggaggctg

SEQ ID NO: 505 - DNA, 25 bp, Homo sapiens
taatcccagctactcgggaggctga

SEQ ID NO: 506 - DNA, 25 bp, Homo sapiens
aatcccagctactcgggaggctgag

SEQ ID NO: 507 - DNA, 25 bp, Homo sapiens
atcccagctactcgggaggctgagg

SEQ ID NO: 508 - DNA, 25 bp, Homo sapiens
tcccagctactcgggaggctgaggc

SEQ ID NO: 509 - DNA, 25 bp, Homo sapiens
cccagctactcgggaggctgaggca

SEQ ID NO: 510 - DNA, 25 bp, Homo sapiens
ccagctactcgggaggctgaggcag

SEQ ID NO: 511 - DNA, 25 bp, Homo sapiens
tggtggctcacgcctgtaatcccag

SEQ ID NO: 512 - DNA, 25 bp, Homo sapiens
gagccgagatcgcgccactgcactc

SEQ ID NO: 513 - DNA, 25 bp, Homo sapiens
gtggctcacgcctgtaatcccagca

SEQ ID NO: 514 - DNA, 25 bp, Homo sapiens
cactgcactccagcctgggcgacag

SEQ ID NO: 515 - DNA, 25 bp, Homo sapiens
actgcactccagcctgggcgacaga

SEQ ID NO: 516 - DNA, 25 bp, Homo sapiens
ctgcactccagcctgggcgacagag

SEQ ID NO: 517 - DNA, 25 bp, Homo sapiens
tgcactccagcctgggcgacagagc

SEQ ID NO: 518 - DNA, 25 bp, Homo sapiens
gcactccagcctgggcgacagagcg

SEQ ID NO: 519 - DNA, 25 bp, Homo sapiens
cactccagcctgggcgacagagcga

SEQ ID NO: 520 - DNA, 25 bp, Homo sapiens
tggctcacgcctgtaatcccagcac

SEQ ID NO: 521 - DNA, 25 bp, Homo sapiens
actccagcctgggcgacagagcgag

SEQ ID NO: 522 - DNA, 25 bp, Homo sapiens
ctccagcctgggcgacagagcgaga

SEQ ID NO: 523 - DNA, 25 bp, Homo sapiens
tccagcctgggcgacagagcgagac

SEQ ID NO: 524 - DNA, 25 bp, Homo sapiens
ccagcctgggcgacagagcgagact

SEQ ID NO: 525 - DNA, 25 bp, Homo sapiens
cagcctgggcgacagagcgagactc

SEQ ID NO: 526 - DNA, 25 bp, Homo sapiens
agcctgggcgacagagcgagactcc

SEQ ID NO: 527 - DNA, 25 bp, Homo sapiens
ggctcacgcctgtaatcccagcact

SEQ ID NO: 528 - DNA, 25 bp, Homo sapiens
gctcacgcctgtaatcccagcactt

SEQ ID NO: 529 - DNA, 25 bp, Homo sapiens
ctcacgcctgtaatcccagcacttt

SEQ ID NO: 530 - DNA, 25 bp, Homo sapiens
tcacgcctgtaatcccagcactttg

SEQ ID NO: 531 - DNA, 25 bp, Homo sapiens
cacgcctgtaatcccagcactttgg

SEQ ID NO: 532 - DNA, 25 bp, Homo sapiens
acgcctgtaatcccagcactttggg

SEQ ID NO: 533 - DNA, 25 bp, Homo sapiens
cgcctgtaatcccagcactttggga

SEQ ID NO: 534 - DNA, 25 bp, Homo sapiens
gcctgtaatcccagcactttgggag

SEQ ID NO: 535 - DNA, 25 bp, Homo sapiens
cctgtaatcccagcactttgggagg

SEQ ID NO: 536 - DNA, 25 bp, Homo sapiens
ctgtaatcccagcactttgggaggc

SEQ ID NO: 537 - DNA, 25 bp, Homo sapiens
tgtaatcccagcactttgggaggcc

SEQ ID NO: 538 - DNA, 25 bp, Homo sapiens
gtaatcccagcactttgggaggccg

SEQ ID NO: 539 - DNA, 25 bp, Homo sapiens
taatcccagcactttgggaggccga

SEQ ID NO: 540 - DNA, 25 bp, Homo sapiens
aatcccagcactttgggaggccgag

SEQ ID NO: 541 - DNA, 25 bp, Homo sapiens
atcccagcactttgggaggccgagg

SEQ ID NO: 542 - DNA, 25 bp, Homo sapiens
tcccagcactttgggaggccgaggt

SEQ ID NO: 543 - DNA, 25 bp, Homo sapiens
cccagcactttgggaggccgaggtg

SEQ ID NO: 544 - DNA, 25 bp, Homo sapiens
gtggatcacctgaggtcaggagttc

SEQ ID NO: 545 - DNA, 25 bp, Homo sapiens
ggatcacctgaggtcaggagttcaa

SEQ ID NO: 546 - DNA, 25 bp, Homo sapiens
gatcacctgaggtcaggagttcaag

SEQ ID NO: 547 - DNA, 25 bp, Homo sapiens
atcacctgaggtcaggagttcaaga

SEQ ID NO: 548 - DNA, 25 bp, Homo sapiens
tcacctgaggtcaggagttcaagac

SEQ ID NO: 549 - DNA, 25 bp, Homo sapiens
aggagttcaagaccagcctggccaa

SEQ ID NO: 550 - DNA, 25 bp, Homo sapiens
ggagttcaagaccagcctggccaac

SEQ ID NO: 551 - DNA, 25 bp, Homo sapiens
gagttcaagaccagcctggccaaca

SEQ ID NO: 552 - DNA, 25 bp, Homo sapiens
agttcaagaccagcctggccaacat

SEQ ID NO: 553 - DNA, 25 bp, Homo sapiens
gttcaagaccagcctggccaacatg

SEQ ID NO: 554 - DNA, 25 bp, Homo sapiens
ttcaagaccagcctggccaacatgg

SEQ ID NO: 555 - DNA, 25 bp, Homo sapiens
tcaagaccagcctggccaacatggt

SEQ ID NO: 556 - DNA, 25 bp, Homo sapiens
caagaccagcctggccaacatggtg

SEQ ID NO: 557 - DNA, 25 bp, Homo sapiens
aagaccagcctggccaacatggtga

SEQ ID NO: 558 - DNA, 25 bp, Homo sapiens
agaccagcctggccaacatggtgaa

SEQ ID NO: 559 - DNA, 25 bp, Homo sapiens
gaccagcctggccaacatggtgaaa

SEQ ID NO: 560 - DNA, 25 bp, Homo sapiens
accagcctggccaacatggtgaaac

SEQ ID NO: 561 - DNA, 25 bp, Homo sapiens
ccagcctggccaacatggtgaaacc

SEQ ID NO: 562 - DNA, 25 bp, Homo sapiens
cagcctggccaacatggtgaaaccc

SEQ ID NO: 563 - DNA, 25 bp, Homo sapiens
tagaattctgtgcagatgtcctgac

SEQ ID NO: 564 - DNA, 25 bp, Homo sapiens
aattctgtgcagatgtcctgacttg

SEQ ID NO: 565 - DNA, 25 bp, Homo sapiens
tgacttggcaattttgtgtccctgc

SEQ ID NO: 566 - DNA, 25 bp, Homo sapiens
ggcaattttgtgtccctgcctcact

SEQ ID NO: 567 - DNA, 25 bp, Homo sapiens
gtcctagtgttgttctgcctcctgt

SEQ ID NO: 568 - DNA, 25 bp, Homo sapiens
ttgttctgcctcctgtcctctcttg

SEQ ID NO: 569 - DNA, 25 bp, Homo sapiens
ctgtcctctcttgctctcttgtcag

SEQ ID NO: 570 - DNA, 25 bp, Homo sapiens
gctctcttgtcagtctctggcttcc

SEQ ID NO: 571 - DNA, 25 bp, Homo sapiens
gtctctggcttcctcggccccattt

SEQ ID NO: 572 - DNA, 25 bp, Homo sapiens
ggccccatttcacttcactgagtcc

SEQ ID NO: 573 - DNA, 25 bp, Homo sapiens
cccatttcacttcactgagtcctga

SEQ ID NO: 574 - DNA, 25 bp, Homo sapiens
tcacttcactgagtcctgacaccca

SEQ ID NO: 575 - DNA, 25 bp, Homo sapiens
aagggtctgttctgctcagctccat

SEQ ID NO: 576 - DNA, 25 bp, Homo sapiens
tgctcagctccatgtcccccatttt

SEQ ID NO: 577 - DNA, 25 bp, Homo sapiens
tttacagcatcctgcactccagcct

SEQ ID NO: 578 - DNA, 25 bp, Homo sapiens
tcctccacaataaaactggggactg

SEQ ID NO: 579 - DNA, 25 bp, Homo sapiens
gctgaggctcccttgcctgactgtg

SEQ ID NO: 580 - DNA, 25 bp, Homo sapiens
gaggctcccttgcctgactgtgact

SEQ ID NO: 581 - DNA, 25 bp, Homo sapiens
ggctcccttgcctgactgtgacttg

SEQ ID NO: 582 - DNA, 25 bp, Homo sapiens
gctcccttgcctgactgtgacttgt

SEQ ID NO: 583 - DNA, 25 bp, Homo sapiens
ctcccttgcctgactgtgacttgtg

SEQ ID NO: 584 - DNA, 25 bp, Homo sapiens
tcccttgcctgactgtgacttgtgc

SEQ ID NO: 585 - DNA, 25 bp, Homo sapiens
cccttgcctgactgtgacttgtgcc

SEQ ID NO: 586 - DNA, 25 bp, Homo sapiens
ccttgcctgactgtgacttgtgcct

SEQ ID NO: 587 - DNA, 25 bp, Homo sapiens
cttgcctgactgtgacttgtgcctc

SEQ ID NO: 588 - DNA, 25 bp, Homo sapiens
ctgactgtgacttgtgcctctctcc

SEQ ID NO: 589 - DNA, 25 bp, Homo sapiens
tgactgtgacttgtgcctctctcct

SEQ ID NO: 590 - DNA, 25 bp, Homo sapiens
gactgtgacttgtgcctctctcctg

SEQ ID NO: 591 - DNA, 25 bp, Homo sapiens
ctgtgacttgtgcctctctcctgcc

SEQ ID NO: 592 - DNA, 25 bp, Homo sapiens
ggtgggcaggtgacccaaggaacct

SEQ ID NO: 593 - DNA, 25 bp, Homo sapiens
caggtgacccaaggaacctttctgg

SEQ ID NO: 594 - DNA, 25 bp, Homo sapiens
tgaaggtactgaacgccacctcact

SEQ ID NO: 595 - DNA, 25 bp, Homo sapiens
aggtactgaacgccacctcactgta

SEQ ID NO: 596 - DNA, 25 bp, Homo sapiens
gtactgaacgccacctcactgtaag

SEQ ID NO: 597 - DNA, 25 bp, Homo sapiens
tgaacgccacctcactgtaagacgg

SEQ ID NO: 598 - DNA, 25 bp, Homo sapiens
aacgccacctcactgtaagacggta

SEQ ID NO: 599 - DNA, 25 bp, Homo sapiens
acgccacctcactgtaagacggtag

SEQ ID NO: 600 - DNA, 25 bp, Homo sapiens
gccacctcactgtaagacggtagat

SEQ ID NO: 601 - DNA, 25 bp, Homo sapiens
ccacctcactgtaagacggtagatt

SEQ ID NO: 602 - DNA, 25 bp, Homo sapiens
acctcactgtaagacggtagatttt

SEQ ID NO: 603 - DNA, 25 bp, Homo sapiens
cctcactgtaagacggtagattttg

SEQ ID NO: 604 - DNA, 25 bp, Homo sapiens
tcactgtaagacggtagattttgta

SEQ ID NO: 605 - DNA, 25 bp, Homo sapiens
gacagggctgccttctgggtgatga

SEQ ID NO: 606 - DNA, 25 bp, Homo sapiens
acagggctgccttctgggtgatgag

SEQ ID NO: 607 - DNA, 25 bp, Homo sapiens
agggctgccttctgggtgatgagaa

SEQ ID NO: 608 - DNA, 25 bp, Homo sapiens
aatcagatgggatggctgcacggcg

SEQ ID NO: 609 - DNA, 25 bp, Homo sapiens
ctgcacggcgtggtgaaggtactga

SEQ ID NO: 610 - DNA, 25 bp, Homo sapiens
ctgcagttcatgtcccccgccaggc

SEQ ID NO: 611 - DNA, 25 bp, Homo sapiens
ccccgccaggcctcgaggctcaggg

SEQ ID NO: 612 - DNA, 25 bp, Homo sapiens
cgccaggcctcgaggctcagggtgg

SEQ ID NO: 613 - DNA, 25 bp, Homo sapiens
gcctcgaggctcagggtgggagagg

SEQ ID NO: 614 - DNA, 25 bp, Homo sapiens
gaggctcagggtgggagagggcccc

SEQ ID NO: 615 - DNA, 25 bp, Homo sapiens
gctcagggtgggagagggccccggg

SEQ ID NO: 616 - DNA, 25 bp, Homo sapiens
ccccgggctgccctgtcactcctct

SEQ ID NO: 617 - DNA, 25 bp, Homo sapiens
cgggctgccctgtcactcctctaac

SEQ ID NO: 618 - DNA, 25 bp, Homo sapiens
gctgccctgtcactcctctaacact

SEQ ID NO: 619 - DNA, 25 bp, Homo sapiens
cctgtcactcctctaacacttccct

SEQ ID NO: 620 - DNA, 25 bp, Homo sapiens
tcactcctctaacacttccctcccg

SEQ ID NO: 621 - DNA, 25 bp, Homo sapiens
ctcctctaacacttccctcccgtgt

SEQ ID NO: 622 - DNA, 25 bp, Homo sapiens
ccccaacatgccctgtaataaaatt

SEQ ID NO: 623 - DNA, 25 bp, Homo sapiens
caacatgccctgtaataaaattaga

SEQ ID NO: 624 - DNA, 25 bp, Homo sapiens
catgccctgtaataaaattagagaa

SEQ ID NO: 625 - DNA, 25 bp, Homo sapiens
tagaatgacccttgggaacagtgaa

SEQ ID NO: 626 - DNA, 25 bp, Homo sapiens
gacccttgggaacagtgaacgtaga

SEQ ID NO: 627 - DNA, 25 bp, Homo sapiens
tttagcagagtttgtgaccaaagtc

SEQ ID NO: 628 - DNA, 25 bp, Homo sapiens
gctctggctgccttctgcatttatt

SEQ ID NO: 629 - DNA, 25 bp, Homo sapiens
gctgccttctgcatttatttgcctt

SEQ ID NO: 630 - DNA, 25 bp, Homo sapiens
gccttggcctgttgtcttcccctat

SEQ ID NO: 631 - DNA, 25 bp, Homo sapiens
gcctgttgtcttcccctattttctg

SEQ ID NO: 632 - DNA, 25 bp, Homo sapiens
tgtcttcccctattttctgtcccag

SEQ ID NO: 633 - DNA, 25 bp, Homo sapiens
ctattttctgtcccagctcatccgt

SEQ ID NO: 634 - DNA, 25 bp, Homo sapiens
ttttctgtcccagctcatccgtgtc

SEQ ID NO: 635 - DNA, 25 bp, Homo sapiens
tctgtcccagctcatccgtgtctct

SEQ ID NO: 636 - DNA, 25 bp, Homo sapiens
gtcccagctcatccgtgtctctgaa

SEQ ID NO: 637 - DNA, 25 bp, Homo sapiens
ccagctcatccgtgtctctgaagaa

SEQ ID NO: 638 - DNA, 25 bp, Homo sapiens
gctcatccgtgtctctgaagaacaa

SEQ ID NO: 639 - DNA, 25 bp, Homo sapiens
ccgtgtctctgaagaacaaatatgc

SEQ ID NO: 640 - DNA, 25 bp, Homo sapiens
ttgccaccctgagcactgcccggat

SEQ ID NO: 641 - DNA, 25 bp, Homo sapiens
ggatcccgtgcaccctgggacccag

SEQ ID NO: 642 - DNA, 25 bp, Homo sapiens
tcccgtgcaccctgggacccagaag

SEQ ID NO: 643 - DNA, 25 bp, Homo sapiens
cgtgcaccctgggacccagaagtgc

SEQ ID NO: 644 - DNA, 25 bp, Homo sapiens
ccgccagcacgtccagagcaactta

SEQ ID NO: 645 - DNA, 25 bp, Homo sapiens
gccagcacgtccagagcaacttacc

SEQ ID NO: 646 - DNA, 25 bp, Homo sapiens
agcacgtccagagcaacttaccccg

SEQ ID NO: 647 - DNA, 25 bp, Homo sapiens
gcacgtccagagcaacttaccccgg

SEQ ID NO: 648 - DNA, 25 bp, Homo sapiens
ccgtgccgccgaccacgatgtgggc

SEQ ID NO: 649 - DNA, 25 bp, Homo sapiens
cgtgccgccgaccacgatgtgggct

SEQ ID NO: 650 - DNA, 25 bp, Homo sapiens
tgccgccgaccacgatgtgggctct

SEQ ID NO: 651 - DNA, 25 bp, Homo sapiens
cgccgaccacgatgtgggctctgag

SEQ ID NO: 652 - DNA, 25 bp, Homo sapiens
gaccacgatgtgggctctgagctgc

SEQ ID NO: 653 - DNA, 25 bp, Homo sapiens
cacgatgtgggctctgagctgcccc

SEQ ID NO: 654 - DNA, 25 bp, Homo sapiens
tgtgaaacgcctagagaccccggcg

SEQ ID NO: 655 - DNA, 25 bp, Homo sapiens
tcacagccccgttcagctggtggct

SEQ ID NO: 656 - DNA, 25 bp, Homo sapiens
ccccgttcagctggtggcttttaga

SEQ ID NO: 657 - DNA, 25 bp, Homo sapiens
ttttagaggcttccagagtgtgctt

SEQ ID NO: 658 - DNA, 25 bp, Homo sapiens
ccagagtgtgcttggcccctttacc

SEQ ID NO: 659 - DNA, 25 bp, Homo sapiens
tggcccctttacctctatgccattg

SEQ ID NO: 660 - DNA, 25 bp, Homo sapiens
ctctatgccattgggcccaggggga

SEQ ID NO: 661 - DNA, 25 bp, Homo sapiens
cctttctgtgtcttgcttgccccgt

SEQ ID NO: 662 - DNA, 25 bp, Homo sapiens
tgtgtcttgcttgccccgtgtctcc

SEQ ID NO: 663 - DNA, 25 bp, Homo sapiens
ttgcttgccccgtgtctcccagtga

SEQ ID NO: 664 - DNA, 25 bp, Homo sapiens
gccccgtgtctcccagtgagtggcc

SEQ ID NO: 665 - DNA, 25 bp, Homo sapiens
tgtctcccagtgagtggccgccctg

SEQ ID NO: 666 - DNA, 25 bp, Homo sapiens
cggacaagtcgcagcctcaggggga

SEQ ID NO: 667 - DNA, 25 bp, Homo sapiens
agtcgcagcctcagggggacctccc

SEQ ID NO: 668 - DNA, 25 bp, Homo sapiens
ctggcactgcatctttctgggcctg

SEQ ID NO: 669 - DNA, 25 bp, Homo sapiens
ctttctgggcctggctctgctgcct

SEQ ID NO: 670 - DNA, 25 bp, Homo sapiens
cagagttataagccccaaacaggtc

SEQ ID NO: 671 - DNA, 25 bp, Homo sapiens
agagttataagccccaaacaggtca

SEQ ID NO: 672 - DNA, 25 bp, Homo sapiens
gagttataagccccaaacaggtcat

SEQ ID NO: 673 - DNA, 25 bp, Homo sapiens
agttataagccccaaacaggtcatg

SEQ ID NO: 674 - DNA, 25 bp, Homo sapiens
gttataagccccaaacaggtcatgc

SEQ ID NO: 675 - DNA, 25 bp, Homo sapiens
ttataagccccaaacaggtcatgct

SEQ ID NO: 676 - DNA, 25 bp, Homo sapiens
tataagccccaaacaggtcatgctc

SEQ ID NO: 677 - DNA, 25 bp, Homo sapiens
ataagccccaaacaggtcatgctcc

SEQ ID NO: 678 - DNA, 25 bp, Homo sapiens
taagccccaaacaggtcatgctcca

SEQ ID NO: 679 - DNA, 25 bp, Homo sapiens
aagccccaaacaggtcatgctccaa

SEQ ID NO: 680 - DNA, 25 bp, Homo sapiens
agccccaaacaggtcatgctccaat

SEQ ID NO: 681 - DNA, 25 bp, Homo sapiens
gccccaaacaggtcatgctccaata

SEQ ID NO: 682 - DNA, 25 bp, Homo sapiens
ccccaaacaggtcatgctccaataa

SEQ ID NO: 683 - DNA, 25 bp, Homo sapiens
cccaaacaggtcatgctccaataaa

SEQ ID NO: 684 - DNA, 25 bp, Homo sapiens
ccaaacaggtcatgctccaataaaa

SEQ ID NO: 685 - DNA, 25 bp, Homo sapiens
cttgcaacctccgggaccatcttct

SEQ ID NO: 686 - DNA, 25 bp, Homo sapiens
gcaacctccgggaccatcttctcgg

SEQ ID NO: 687 - DNA, 25 bp, Homo sapiens
gcttctgggacctgccagcaccgtt

SEQ ID NO: 688 - DNA, 25 bp, Homo sapiens
gggacctgccagcaccgtttttgtg

SEQ ID NO: 689 - DNA, 25 bp, Homo sapiens
tgccagcaccgtttttgtggttagc

SEQ ID NO: 690 - DNA, 25 bp, Homo sapiens
cagcaccgtttttgtggttagctcc

SEQ ID NO: 691 - DNA, 25 bp, Homo sapiens
ttgccaaccaaccatgagctcccag

SEQ ID NO: 692 - DNA, 25 bp, Homo sapiens
gccaaccaaccatgagctcccagat

SEQ ID NO: 693 - DNA, 25 bp, Homo sapiens
aaccaaccatgagctcccagattcg

SEQ ID NO: 694 - DNA, 25 bp, Homo sapiens
ccatgagctcccagattcgtcagaa

SEQ ID NO: 695 - DNA, 25 bp, Homo sapiens
tgagctcccagattcgtcagaatta

SEQ ID NO: 696 - DNA, 25 bp, Homo sapiens
gctcccagattcgtcagaattattc

SEQ ID NO: 697 - DNA, 25 bp, Homo sapiens
cccagattcgtcagaattattccac

SEQ ID NO: 698 - DNA, 25 bp, Homo sapiens
gattcgtcagaattattccaccgac

SEQ ID NO: 699 - DNA, 25 bp, Homo sapiens
tcgtcagaattattccaccgacgtg

SEQ ID NO: 700 - DNA, 25 bp, Homo sapiens
tcagaattattccaccgacgtggag

SEQ ID NO: 701 - DNA, 25 bp, Homo sapiens
actggcatggccttccgtgtcccca

SEQ ID NO: 702 - DNA, 25 bp, Homo sapiens
ccactgccaacgtgtcagtggtgga

SEQ ID NO: 703 - DNA, 25 bp, Homo sapiens
actgccaacgtgtcagtggtggacc

SEQ ID NO: 704 - DNA, 25 bp, Homo sapiens
tgccaacgtgtcagtggtggacctg

SEQ ID NO: 705 - DNA, 25 bp, Homo sapiens
ccaacgtgtcagtggtggacctgac

SEQ ID NO: 706 - DNA, 25 bp, Homo sapiens
cgtgtcagtggtggacctgacctgc

SEQ ID NO: 707 - DNA, 25 bp, Homo sapiens
gtcagtggtggacctgacctgccgt

SEQ ID NO: 708 - DNA, 25 bp, Homo sapiens
cagtggtggacctgacctgccgtct

SEQ ID NO: 709 - DNA, 25 bp, Homo sapiens
gtggtggacctgacctgccgtctag

SEQ ID NO: 710 - DNA, 25 bp, Homo sapiens
ggtggacctgacctgccgtctagaa

SEQ ID NO: 711 - DNA, 25 bp, Homo sapiens
gacctgacctgccgtctagaaaaac

SEQ ID NO: 712 - DNA, 25 bp, Homo sapiens
ctgacctgccgtctagaaaaacctg

SEQ ID NO: 713 - DNA, 25 bp, Homo sapiens
gacctgccgtctagaaaaacctgcc

SEQ ID NO: 714 - DNA, 25 bp, Homo sapiens
tgccgtctagaaaaacctgccaaat

SEQ ID NO: 715 - DNA, 25 bp, Homo sapiens
ccgtctagaaaaacctgccaaatat

SEQ ID NO: 716 - DNA, 25 bp, Homo sapiens
gtctagaaaaacctgccaaatatga

SEQ ID NO: 717 - DNA, 2611 bp, Homo sapiens
aggaacgactgtgctacgttgccagaaggggcgggacctgcaacgtccgacagaacgaggggacgtaacggaggcaggttggagccgctgccgtcgccatgacccgcggtaaccagcgtgagctcgcccgccagaagaatatgaaaaagcagagcgactcggttaagggaaagcgccgagatgacgggctttctgctgccgcccgcaagcagagggactcggagatcatgcagcagaagcagaaaaaggcaaacgagaagaaggaggaacccaagtagctttgtggcttcgtgtccaaccctcttgcccttcgcctgtgtgcctggagccagtcccaccacgctcgcgtttcctcctgtagtgctcacaggtcccagcaccgatggcattccctttgccctgagtctgcagcgggtcccttttgtgcttccttcccctcaggtagcctctctccccctgggccactcccgggggtgagggggttaccccttcccagtgttttttattcctgtggggctcaccccaaagtattaaaagtagctttgtaattccttgagcgcctggtttgactggggacttggggggatggggttggaagaatgactgccctttcccaccaaaaaagggagaactctttagattcagattgtgggtatgtagacttaataagtgaaacatcacagaagaagcctttattatacaatgacaaccaaacaagtactccggatatgcagtagaggaatcctctaagaaccatagagacttcttttctgtgatttttgttccccacccttgaacaccatctctaggatggagttggcctaagagtgaatgctgcaagatctgtgtttatgcctcttttcctcattcttcctcagtttgttcgtctgcttgaaagttggccaaaaaatcctgctgctcaccgacttcccgtggtcagctgctgtcaagcgttcactttctcttctgtcattcctcatggaatgagggtggttttgtcttcccgcttcccttgacctcaaaatcaggattaaaacctggggtagcctctgtgctcctttcttctatgccctggtttgttctgtggttctgggcttcttatatccgtgtgcccagggctgaactccttattttcctttctccaagggcagagccgagtcttcagtccctgttggtctttccccacccccacttccagcccaagagccaggaaagggctggtgccacactgtctgctgggatcagcggtggttctttgagctgctgatttgggtgttaggctcttgagctgggatgcagatgtaacagtagctccagtgagtcagacactctgcccagcacattagactgtgtttgaccacttcttccagttcatagtattgacttcagcccaaacggagataactccctgtgtgtccttgaggtattgagctgggctggacagctccccttgagccaactctaggagtacaatgtcaggggaaccccagtttgtgaaaaggacttagactggaggatatttgttatctggggatatgatgcggtggcggcggcgcctcaagataaggggctggggtttctgggtggggggccaacagagtggtgccagtaacagccccagatagaggagtacgcaggcccagcatgaggcaaccttgacccagaaggtggcccagctacccttgatgaaggtcttttccagttctgctccctcatagctgtgtaaccaaaggctctggttagagaatatgaagggccttagcttttagacctgttctacctcctcaccaaatataatggcagacccatgtgtgtctggaatggccttgaattgctctttccttaaaatagctagctcttcaggagagtatctaaggcccactccatcttacctgaaccagttggtaagggtaaccatgacatagagtgaggcaaggaagaagacgaagtggaaggcagaatagttgtaggaaagatgctggacttggactggaggagctggaggggtttcttggtcagctggcctcgcagccccacccctttgccctggagagaggaaatggctgctgggagcagagctgctgaaacacctcttcccctctcccccaactacctttgttaaggctcttgagggttcttatggcactccacagagatctaccacttcttatggttcctcacttggcactcacctttgtctgcctccactgtttcagggcagcagaaacacagtgagggcttctgcaaaacagaacgcaggttttggaatggtcttaaaagatgtgagggtgttaatctaggaaacttcccccgtgaaaagattggtctagtattaaaaagtggaggcacacctgggttcaaattctagctccagcatataagtggctgtgcagactttggtaagatgtttaatcttttgtgcctcgatttctccatttgtaaaatggagcaaatacctacctcacagggttgttgtgagggttaaattaaatgagattatgtaaaagtatctagcacagttgcctagcacattgtgggtactcaataaaaggtaacagcagctataatctgagcattctgggtagaggttggtaaaaaaaaaaaaaaaaaa

SEQ ID NO: 718 - DNA, 3432 bp, Homo sapiens
actacttctgcgcctgcgcgaccgtgattccccgctcgcgactccccaccccccagggctccctaaagagggccacgagctgcgaaagggcgggaaaggcagttggagaagaggtaagcggttactcactccatggctgcagcaaggagaggcggcggcggcctcggctgaagaaagaaggtgggagcggagagcgcaggcgtgccgaggtggatgtccgtcttttctctgttgcagaaacccaccttgtcccatccacatcaggacatcccagctggagttcaaccttcatcccttctgtggcagttaggagactgaatcaaggtccagagaaggtggaggaatcctgatactgagcgaaatcttcccaaggctgcagacaccgacggatttgctttgggagccagagtagctgccgccaccagagtccggagccatgagcgggtttaattttggaggcactggggcccctacaggcgggttcacgtttggcactgcaaagacggcaacaaccacacctgctacagggttttctttctccacctctggcactggagggtttaattttggggctcccttccaaccagccacaagtaccccttccaccggcctgttctcacttgccacccagactccggccacacagacgacaggcttcacttttggaacagcgactcttgcttcggggggaactggattttctttggggatcggtgcttcaaagctcaacttgagcaacacagctgccaccccagccatggcaaaccccagcggctttgggctgggcagcagcaacctcactaatgccatatcgagcaccgtcacctccagccagggcacagcacccaccggctttgtgtttggcccctccaccacctctgtggctccagctaccacatctggaggcttctcattcactggtggaagcacggcccaaccctccggtttcaacattggctcagcagggaattcagcccagcccacggcacctgccacgttgcccttcactccggccacgccagcagccaccacagcaggtgccacacagccagctgctcccacacccacagccaccatcaccagtactgggcccagcctctttgcgtcaatagcaactgctccaacctcatctgccaccactggactctccctctgtacccctgtgaccacagcgggcgcccccactgctgggacacagggcttcagcttaaaggcacctggagcagcttccggcacctccacaacaacatccaccgctgccaccgccaccgccaccaccaccagcagcagcagcaccaccggctttgccttgaatttaaaaccactggcgccagccgggatccccagcaatacagcagctgccgtgaccgctccacctggccctggcgcagctgcaggggcggctgccagctccgccatgacctacgcgcagctggagagcctgatcaacaaatggagcctggagctagaggaccaggagcggcacttcctccagcaggccacccaggtcaacgcctgggaccgcacgctgatcgagaatggagaaaagatcaccagcctgcaccgcgaggtggagaaggtgaagctggaccagaagaggctggaccaggagctcgacttcatcctgtcccagcagaaggagctggaagacctgctgagcccactggaggagttggtcaaggagcagagcgggaccatctacctgcagcacgcggatgaggagcgtgagaaaacctacaagctggctgagaacatcgacgcacagctcaagcgcatggcccaggatctcaaggacatcatcgagcacctgaacacgtccggggcccccgccgacaccagtgacccactgcagcagatctgcaagatcctcaatgcgcacatggactcactgcagtggatcgaccagaactcggccctgctgcagaggaaggtggaggaggtgaccaaggtgtgcgagggccggcgcaaggagcaggagcgcagcttccggatcacctttgactgagcgacagcagccctggggcccgcaggtccctagggagttcatgaggggaatgcgccctgttgtctgtagtttggggttgtggcaagatacttgtttgtttctttctttctttcacatgactgcccttgacatgatcgctgtgtgctttgcgtttttccatttaggagggtattctgggccttctgcccaggcagcagcctcatgggtgtggcttctgtggctttcatttgagtatctttggccccttttcacctactgcgaccacccacctcatcctggctcagcctggtgatggagaagtgctgatggtcttggtcccagccagggtcgtgggggcagccactctctccaaagcatagtcataggtgtcatgaaaaaataccaaatgtaagagaacctccaagtcagggcgcagtggctcacccctgtaatctcagcactttgggtggccaaggcgggcagatgacttgaggtcaggagttcgagaccagcctggccaacatggtgaaaccccgtctctactaaaaatacaaaaattagtcaggtgtggtggacgcctgtgatctcaatctcagctactcgggaggctgaggcaggagaatcacttgaacccaggaggtgttgcagtgaaccaagatcacaccactgcactccagcctaggcaacagagactctgtctcaaaaaaaaaaaaaaaaaaaaagaaactcccaggagacagcagcctagttttcgagtgtgagcttgtgcttgtgaaagctaaccatgctaaccaccaaggcaaagcagcacagtgtgaatagaacagagcgggatcaagaatttcacagaagacaggtcagctgaggggcctgcacacacagggtgttgaggaaccacagatgggcgccgagaggcctgccttttgcctggcccaggctcacccccaccttgggcctcacctcctccaggaagccttcccagctacccgaagctcaggtggccttcttgcaggtccccgtagcaccctgagcctgtaccttgggtggcacttgttatgctatcctgtgctagccgtttgtgcctcgtctcgctgttagattgtgagttcccatgggcagagacccactgtcgttccccgtgtgtccccagcccggtccctgtcacatttgttaaatgaaagaacaatgaagcccagtgtaacgtcagtccacagaaatagccacagcttccagtggtggccgtagacttggctcggaacttagtggcaccagagtaactctagtcagttacagtaaaatccactgtgtgtggaaggcagaagctagcggttgtatcccaagcatcttttgtatttgtctttatactttgctgaattctctgaaatacctattactgtatgttgcttttctaaataaatgtattgtgaaaccaaaaaaaaaaaaaaaaa

SEQ ID NO: 719 - DNA, 1986 bp, Homo sapiens
aaggccccgctgcgtcttccgagccgcaggcgcaggcccagctgagcggccgccgagcgggtgcgggtgcgggcgcatcggccatcaccgcgcggccgcgcagcggacaccgtgcgtaccggcctgcggcgcccggccaccggtgagtccccggcccgagcccaggagcgcctctgacccgctgcgccgcgcggcctgccgcccccgcccccgcccccacgcggatcttgcgcatccgagcgtggccgcctcgggggcggaccgcggaacccgaggccatgtcccatgaaaagagttttttggtgtctggggacaactatcctccccccaaccctggatatccgggggggccccagccacccatgcccccctatgctcagcctccctaccctggggccccttacccacagccccctttccagccctccccctacggtcagccagggtacccccatggccccagcccctacccccaagggggctacccacagggtccctacccccaagggggctacccacagggcccctacccacaagagggctacccacagggcccctacccccaagggggctacccccaggggccatatccccagagccccttcccccccaacccctatggacagccacaggtcttcccaggacaagaccctgactcaccccagcatggaaactaccaggaggagggtcccccatcctactatgacaaccaggacttccctgccaccaactgggatgacaagagcatccgacaggccttcatccgcaaggtgttcctagtgctgaccttgcagctgtcggtgaccctgtccacggtgtctgtgttcacttttgttgcggaggtgaagggctttgtccgggagaatgtctggacctactatgtctcctatgctgtcttcttcatctctctcatcgtcctcagctgttgtggggacttccggcgaaagcacccctggaaccttgttgcactgtcggtcctgaccgccagcctgtcgtacatggtggggatgatcgccagcttctacaacaccgaggcagtcatcatggccgtgggcatcaccacagccgtctgcttcaccgtcgtcatcttctccatgcagacccgctacgacttcacctcatgcatgggcgtgctcctggtgagcatggtggtgctcttcatcttcgccattctctgcatcttcatccggaaccgcatcctggagatcgtgtacgcctcactgggcgctctgctcttcacctgcttcctcgcagtggacacccagctgctgctggggaacaagcagctgtccctgagcccagaagagtatgtgtttgctgcgctgaacctgtacacagacatcatcaacatcttcctgtacatcctcaccatcattggccgcgccaaggagtagccgagctccagctcgctgtgcccgctcaggtggcacggctggcctggaccctgcccctggcacggcagtgccagctgtacttcccctctctcttgtccccaggcacagcctagggaaaaggatgcctctctccaaccctcctgtatgtacactgcagatacttccatttggacccgctgtggccacagcatggcccctttagtcctcccgcccccgccaaggggcaccaaggccacgtttccgtgccacctcctgtctactcattgttgcatgagccctgtctgccagcccaccccagggactgggggcagcaccaggtcccggggagagggattgagccaagaggtgagggtgcacgtcttccctcctgtcccagctccccagcctggcgtagagcacccctcccctcccccccacccccctggagtgctgccctctggggacatgcggagtgggggtcttatccctgtgctgagccctgagggcagagaggatggcatgtttcaggggagggggaagccttcctctcaatttgttgtcagtgaaattccaataaatgggatttgctctctgcaaaaaaaaaaaaaaaaaa

SEQ ID NO: 720 - DNA, 3973 bp, Homo sapiens
cggacggggccgccccgatgggacgccgcgctccggcccctgcgcgccgctgagccgagcgccccccgctgccgagacccccgccgccaccgccagccgctgccccctcgcccccgcccgggccgggagcctcgtccccgtcccccggaaagctggatttccgaggctggaggcgcctggccggctgggtggggaccaccatgggcaacgcggccggcagcgccgagcagcccgcgggccccgccgcgccgccccccaagcagcccgcgcctcccaagcagccgatgcccgcggccggagagctggaggagaggttcaaccgcgccctgaactgcatgaacttgcccccagacaaggtccagctgctgagccagtatgacaacgagaagaagtgggagctcatctgtgatcaggagcggtttcaagtcaagaatccccccgcagcctacatccagaagctgaagagctatgtggatactggtggggtcagccgaaaggtagcagctgattggatgtccaacctggggtttaagaggcgagttcaggagtccacgcaggtgctacgggagctggagacctccctgaggaccaaccacattgggtgggtgcaggagttcctcaatgaagagaaccgtggcctggatgtgctgctcgagtacctggcctttgcccagtgctctgtcacgtatgacatggagagcacagacaacggggcttccaactcagagaaaaacaagcccctggagcagtctgtggaagacctcagcaagggtccaccctcctccgtgcccaaaagccgccacctgaccatcaagctgaccccagcccacagcaggaaggccctgcggaattcccgcatcgtcagccagaaggacgacgtccacgtctgtattatgtgcctacgcgccatcatgaactaccagtctggcttcagccttgtcatgaaccacccagcctgtgtcaatgagattgctctgagcctcaacaacaagaaccccagaaccaaggctctggtgctggagctgctggcggccgtgtgcttggtgcggggaggacatgacatcatccttgcagcctttgacaacttcaaggaggtgtgtggggagcagcaccgctttgaaaagctgatggaatatttccggaatgaggacagcaacatcgacttcatggtggcctgcatgcagttcatcaacattgtggtacattcggtggagaacatgaacttccgtgtcttcctgcaatatgagttcacccacttgggcctggacctgtacttggagaggcttcggctcaccgagagtgacaagctgcaggtgcagatccaggcgtacctggacaatatttttgatgtgggggcgctgctggaggacacagagaccaagaacgctgtgctggagcacatggaggaactgcaggagcaagtggcgctgctgacagagcggcttcgggacgcggagaacgaatccatggccaagattgcagaactggaaaaacagctaagccaggcgcgcaaggagttggagaccctgcgggagcgcttcagcgaatcgaccgccatgggcgcctccaggcgtcccccagagcctgagaaagcgcctcccgctgccccgacgcggccctcggccctggagctgaaggtggaggagctggaggagaaggggttaatccgtattctgcgggggccgggggatgctgtctccatcgagatcctccccgtcgctgtggcaactccgagcggcggtgatgctccgactccgggggtgccgaccggctcccccagcccagatctcgcacctgcagcagagccggctcccggagcagcgccaccgccgccgcccccactgcccggcctcccctccccgcaggaagccccgccctctgcgcccccacaggccccgcctctccctggcagcccggagcccccgcctgcgccgccgctgcccggagacctgccgcccccacccccgccaccgccaccacctccgggcactgacgggccggtgcctccgccgccgccgccgccgccgccgcctcccggaggtcctcctgatgccctaggaagacgcgactcagaattgggcccaggagtgaaggccaagaagcccatccagactaagttccgaatgccactcttgaactgggtggcactgaaacccagccagatcaccggcactgtcttcacagagctcaatgatgagaaggtgctgcaggagctagacatgagtgattttgaggaacagttcaagaccaagtcccaaggccccagcctggacctcagcgctctcaagagtaaggcagcccagaaggcccccagcaaggcgacactcattgaggccaaccgggccaagaacttggccatcaccctgcggaagggcaacctgggggccgagcgcatctgccaagccattgaggcgtacgacctgcaggctctgggcctggacttcctggagctgctgatgcgcttcctgcccacagagtatgagcgcagcctcatcacccgctttgagcgggagcagcggccaatggaggagctgtcagaggaggaccgcttcatgctatgcttcagccgcatcccgcgcctgccggagcgcatgaccacactcaccttcctgggcaacttcccggacacagcccagctgctcatgccgcaactgaatgccatcattgcagcctcaatgtccatcaagtcctctgacaaactccgccagatcctggagattgtcctggcctttggcaactacatgaacagtagcaagcgtggggcagcctatggcttccggctccagagcctggatgcgctgttggagatgaagtcgactgatcgcaagcagacgctgctgcactacctggtgaaggtcattgctgagaagtacccgcaactcacaggcttccacagcgacctgcacttcctggacaaggcgggctcagtgtccctggacagtgtcctggcggacgtgcgctccctgcagcgaggcctagagttgacacagagagagtttgtgcggcaggatgactgcatggtgctcaaggagttcctgagggccaactcgcccaccatggacaagctgctggcagacagcaagacggctcaggaggcctttgagtctgtggtggagtacttcggagagaaccccaagaccacatccccaggcctgttcttctccctctttagccgcttcattaaggcctacaagaaagctgagcaggaggtggaacagtggaaaaaagaagccgctgcccaggaggcaggcgctgataccccgggcaaaggggagcccccagcacccaagtcaccgccaaaggcccggcggccacagatggacctcatctctgagctgaaacggaggcagcagaaggagccactcatttatgagagcgaccgtgatggggccattgaagacatcatcacagtgatcaagacggtgcccttcacggcccgcaccggcaagcggacatcccggctcctctgtgaggccagcctgggagaagagatgcccctctagcccctcagatctgcggaaccagccctacatccgcgcagacacaggccgccgcagtgcccgtcggcgtcccccgggccccccactgcaggtcacctccgacctctcgctgtagccgctatttctgcaggtggattctgcaggggtgtggggccgtggacaggctgaggctcaaggaaggtggtcctcagctcggctggccgggcagcccctcctccgctgtggcccgcctcaaacgggctggtgcatcctcctcttggccacagagggcagcatcgcccgccccttcccccaaatgctgcttgcagcacccaccctaaagccccctccaaatagccatacttagcctcagcaggagcctggcctgtaacttataaagtgcacctcgcccccgcaagccccagccccgaggaccgtccatggaccttatttttatatgagattaataaagatgtttgcaaaaaaaaaaaaaa

SEQ ID NO: 721 - DNA, 5532 bp, Homo sapiens
ggcccaccgccgcccaggcaaggccgccctgccttgggcgcagcgctgccatggctgggggccgtggggcccccgggcgcggccgggacgagcctccggagagctacccgcaacgacaggaccacgagctacaggccctggaggccatctacggcgcggacttccaagacctgcggccggacgcttgcggaccggtcaaagagccccctgaaatcaatttagttttgtaccctcaaggcctaactggtgaagaagtatatgtaaaagtggatttgagggttaaatgcccacctacctatccagatgtagttcctgaaatagagttaaaaaatgccaaaggtctatcaaatgaaagtgtcaatttgttaaaatctcgcctagaagaactggccaagaaacactgtggggaggtgatgatctttgaactggcttaccacgtgcagtcatttctcagcgagcataacaagccccctcccaagtcttttcatgaagaaatgctggaaaggcgggctcaggaggagcagcagaggctgttggaggccaagcggaaagaagagcaggagcaacgtgaaatcctgcatgagattcagagaaggaaagaagagataaaagaagagaaaaaaaggaaagaaatggctaagcaggaacgtttggaaattgctagtttgtcaaaccaagatcatacctctaagaaggacccaggaggacacagaacggctgccattctacatggaggctctcctgactttgtaggaaatggtaaacatcgggcaaactcctcaggaaggtctaggcgagaacgtcagtattctgtatgtaatagtgaagattctcctggctcttgtgaaattctgtatttcaatatggggagtcctgatcagctcatggtgcacaaagggaaatgtattggcagtgatgaacaacttggaaaattagtctacaatgctttggaaacagccactggtggctttgtcttgttgtatgagtgggtccttcagtggcagaaaaaaatgggtccattccttaccagtcaagaaaaagagaagattgataagtgcaaaaagcagattcaaggaacagaaacagaattcaactcactggtaaaattgagccatccaaatgtagtacgctaccttgcaatgaatctcaaagagcaagacgactccatcgtggtggacattttagtggagcacattagtggggtctctcttgctgcacacctgagccactcaggccccatccctgtgcatcagcttcgcaggtacacagctcagctcctgtcaggccttgattatctgcacagcaattctgtggtgcataaggtcctgagtgcatctaatgtcttggtggatgcagaaggcaccgtcaagattacggactatagcatttctaagcgcctcgcagacatttgcaaggaggatgtgtttgagcaaacccgagttcgttttagtgacaatgctctgccttataaaacggggaagaaaggagatgtttggcgtcttggccttctgctgctgtccctcagccaaggacaggaatgtggagagtaccctgtgaccatccctagtgacttaccagctgactttcaagattttctaaagaaatgtgtgtgcttggatgacaaggaaagatggagtccccagcagttgttgaaacacagctttataaatccccagccaaaaatgcctctagtggaacaaagtcctgaagattctgaaggacaagattatgttgagactgttattcctagcaaccggctacccagtgctgccttctttagtgagacacagagacagttttcccgatacttcattgagtttgaagaattacaacttcttggtaaaggagcttttggagctgtcatcaaggtgcagaacaagttggacggctgctgctacgcagtgaagcgcatccccatcaacccggccagccggcagttccgcaggatcaagggcgaagtgacactgctgtcacggctgcaccatgagaacattgtgcgctactacaacgcctggatcgagcggcacgagcggccggcgggaccggggacgccgcccccggactccgggcccctggccaaggatgaccgagctgcacgcgggcagccggcgagcgacacagacggcctggacagcgtagaggccgccgcgccgccacccatcctcagcagctcggtggagtggagcacttcgggcgagcgctcggccagtgcccgtttccccgccaccggcccgggctccagcgatgacgaggacgacgacgaggacgagcacggtggcgtcttctcccagtccttcctgcctgcttcagattctgaaagtgatattatctttgacaatgaagatgagaacagtaaaagtcagaatcaggatgaagattgcaatgaaaagaatggctgccatgaaagtgagccatcagtgacgactgaggctgtgcactacctatacatccagatggagtactgtgagaagagcactttacgagacaccattgaccagggactgtatcgagacaccgtcagactctggaggctttttcgagagattctggatggattagcttatatccatgagaaaggaatgattcaccgggatttgaagcctgtcaacatttttttggattctgatgaccatgtgaaaataggtgattttggtttggcgacagaccatctagccttttctgctgacagcaaacaagacgatcagacaggagacttgattaagtcagacccttcaggtcacttaactgggatggttggcactgctctctatgtaagcccagaggtccaaggaagcaccaaatctgcatacaaccagaaagtggatctcttcagcctgggaattatcttctttgagatgtcctatcaccccatggtcacggcttcagaaaggatctttgttctcaaccaactcagagatcccacttcgcctaagtttccagaagactttgacgatggagagcatgcaaagcagaaatcagtcatctcctggctgttgaaccacgatccagcaaaacggcccacagccacagaactgctcaagagtgagctgctgcccccaccccagatggaggagtcagagctgcatgaagtgctgcaccacacgctgaccaacgtggatgggaaggcctaccgcaccatgatggcccagatcttctcgcagcgcatctcccctgccatcgattacacctatgacagcgacatactgaagggcaacttctcaatccgtacagccaagatgcagcagcatgtgtgtgaaaccatcatccgcatctttaaaagacatggagctgttcagttgtgtactccactactgcttccccgaaacagacaaatatatgagcacaacgaagctgccctattcatggaccacagcgggatgctggtgatgcttccttttgacctgcggatcccttttgcaagatatgtggcaagaaataatatattgaatttaaaacgatactgcatagaacgtgtgttcaggccgcgcaagttagatcgatttcatcccaaagaacttctggagtgtgcatttgatattgtcacttctaccaccaacagctttctgcccactgctgaaattatctacactatctatgaaatcatccaagagtttccagcacttcaggaaagaaattacagtatttatttgaaccataccatgttattgaaagcaatactcttacactgtgggatcccagaagataaactcagtcaagtctacattattctgtatgatgctgtgacagagaagctgacgaggagagaagtggaagctaaattttgtaatctgtctttgtcttctaatagtctgtgtcgactctacaagtttattgaacagaagggagatttgcaagatcttatgccaacaataaattcattaataaaacagaaaacaggtattgcacagttggtgaagtatggcttaaaagacctagaggaggttgttggactgttgaagaaactcggcatcaagttacaggtcttgatcaatttgggcttggtttacaaggtgcagcagcacaatggaatcatcttccagtttgtggctttcatcaaacgaaggcaaagggctgtacctgaaatcctcgcagctggaggcagatatgacctgctgattccccagtttagagggccacaagctctggggccagttcccactgccattggggtcagcatagctatagacaagatatctgctgctgtcctcaacatggaggaatctgttacaataagctcttgtgacctcctggttgtaagtgttggccagatgtctatgtccagggccatcaacctaacccagaaactctggacagcaggcatcacagcagaaatcatgtacgactggtcacagtcccaagaggaattacaagagtactgcagacatcatgaaatcacctatgtggcccttgtctcggataaagaaggaagccatgtcaaggttaagtctttcgagaaggaaaggcagacagagaagcgtgtgctggagactgaacttgtggaccatgtactgcagaaactgaggactaaagtcactgatgaaaggaatggcagagaagcttccgataatcttgcagtgcaaaatctgaaggggtcattttctaatgcttcaggtttgtttgaaatccatggagcaacagtggttcccattgtgagtgtgctagccccggagaagctgtcagccagcactaggaggcgctatgaaactcaggtacaaactcgacttcagacctcccttgccaacttacatcagaaaagcagtgaaattgaaattctggctgtggatctacccaaagaaacaatattacagtttttatcattagagtgggatgctgatgaacaggcatttaacacaactgtgaagcagctgctgtcacgcctgccaaagcaaagatacctcaaattagtctgtgatgaaatttataacatcaaagtagaaaaaaaggtgtctgtgctatttctgtacagctatagagatgactactacagaatcttattttaaccctaaagaactgtcgttaacctcattcaaacagacagaggcttatactggaataatggaatgttgtacattcatcataatttaaaattaaattctaagaagaggctgggtgcagtggctcacacctttaatcccagcactttgggaagccaaggcaggaagactgcttgaaaccaggagtttgagaccagcctgagcaacaaagcaagaccccatctctataaaaactaaaaaaattagttgggcatggtggcacatgcctgtagtcccagctactccagaggctgagatggatcatctgagcctcaggaggttgaggctgcagtgagctgtgactgcgccactgcactccagtctgggacaacagagcaagaccctgtcttaaaaaaaaaaagaaaaaaaaaatttttttctaagaagctgtcctacaaagttgagctttgttagtttttcatgtgtaatatattataaatttatcttttgggatataataaatgctttcatatacctgcaaaaaaaaaaaaaaaaaaaa

SEQ ID NO: 722 - DNA, 736 bp, Homo sapiens
cccttccggctggccccgctcagtcacccgcagcaggcgtgcagtttcccggctctccgcgcggccggggaaggtcagcgccgtaatggcgttcttggcgtcgggaccctacctgacccatcagcaaaaggtgttgcggctttataagcgggcgctacgccacctcgagtcgtggtgcgtccagagagacaaataccgatactttgcttgtttgatgagagcccggtttgaagaacataagaatgaaaaggatatggcgaaggccacccagctgctgaaggaggccgaggaagaattctggtaccgtcagcatccacagccatacatcttccctgactctcctgggggcacctcctatgagagatacgattgctacaaggtcccagaatggtgcttagatgactggcatccttctgagaaggcaatgtatcctgattactttgccaagagagaacagtggaagaaactgcggagggaaagctgggaacgagaggttaagcagctgcaggaggaaacgccacctggtggtcctttaactgaagctttgccccctgcccgaaaggaaggtgatttgcccccactgtggtggtatattgtgaccagaccccgggagcggcccatgtagaaagagagagacctcatctttcatgcttgcaagtgaaatatgttacagaacatgcacttgccctaataaaaaatcagtgaaatggtctctggtaaaaaaaaaaaaaaaaaa

SEQ ID NO: 723 - DNA, 1968 bp, Homo sapiens
gaggaggaggaggagatgactggggagcgggagctcgagaatactgcccagttactctagcgcgccaggccgaaccgcagcttcttggcttaggtacttctactcacagcggccgattccgaggccaactccagcaatggcttttgcaaatctgcggaaagtgctcatcagtgacagcctggacccttgctgccggaagatcttgcaagatggagggctgcaggtggtggaaaagcagaaccttagcaaagaggagctgatagcggagctgcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggagcaaggcttcggggaatgcctcctggccgtggccctggcaggcgccccttaccaggctgtgggcttggtccaaggcactacacctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaaccttggagctcactggtccctgcctctggggcttttctgaagaaacccacccactgtgatcaatagggagagaaaatccacattcttgggctgaacgcgggcctctgacactgcttacactgcactctgaccctgtagtacagcaataaccgtctaataaagagcctacccccaaaaaaaaaaaaaaaaaaaaaaaaaaaaaaaaaaaaaaaaaaaaaaaaaaaaaaaaa

SEQ ID NO: 724 - DNA, 3524 bp, Homo sapiens
gagtcgcgggccttttgagggaggaggcagagcgcgccgggccggtggcatcttccttactttgtccatcctccggactcgcgatcttccttccggagccatgtcagaaggagtggacttgattgatatatatgctgacgaggagttcaaccaggacccagagttcaacaatacagatcagattgacctgtatgatgatgtgctgacagccacctcacagccctcagatgacagaagcagcagcactgaaccacctcctcctgttcgccaggagccatctcccaagcccaacaacaagacccctgcaattctgtatacctacagtggcctgcgtaatagacgagctgccgtttatgtgggcagcttctcctggtggaccacagaccagcagctgatccaggttattcgctctataggagtctatgatgtggtggagttgaaatttgcagagaatcgagcaaatggccagtccaaagggtatgctgaggtggtggtagcctctgaaaactctgtccacaaattgttggaactcctaccagggaaagttcttaatggagaaaaagtggacgtgaggccggccacccggcagaacctgtcacagtttgaggcacaggctcggaaacgtgagtgtgtccgagtcccaagagggggaatacctccacgggcccattcccgagattctagtgattctgctgatggacgggccacaccctctgagaaccttgtaccctcatctgctcgtgtggataagccccccagtgtgctgccctacttcaatcgtcctccttcggcccttcccctgatgggtctgcccccaccaccaattccacccccaccacctctctcctcaagctttggggtccctcctcctcctcctggtatccactaccagcatctcatgcccccacctcctcgattacctcctcatcttgctgtacctccccctggggccatcccacctgcccttcacctcaatccagccttcttccccccaccaaacgctacagtggggcctccaccagatacttacatgaaggcctctgccccctataaccaccatggcagccgagattcgggccctccaccctctacagtgagtgaagccgaatttgaagatatcatgaagcgaaacagagcaatttccagcagtgccatttccaaagcagtatctggagccagtgcaggggattacagtgacgcaattgagacgctgctcacagccattgcggttatcaaacagtcccgggttgccaatgatgagcgttgccgtgtcctcatctcctctcttaaggactgtcttcatggcattgaagccaagtcctacagtgtgggtgccagtgggagctcttccaggaaaagacatcgctcccgggaaaggtcacctagccggtcccgggagagcagcaggaggcaccgggatctgcttcataatgaagatcggcatgatgattatttccaagaaaggaaccgggagcatgagagacaccgggatagagaacgggaccggcaccactgagaaaggagtctggttggaagcaaatgtttttttaatggacttgcatctcctcaccttgatcaggactaaaggacggaggccgccccacccccttccctttcctccaaacccctaactccctccagacacccagggaataccctctgccccacaggattgaagactgcttggcagtcctcccaatcccacacctcctgtttgccaggggaaagaacctaaagacttcgtgtgattgggaggggtggcagacaggaagaaaacatgtccaggcccctggtctccatagagaatggtgctttgtccaagaaaacgtatgagtttctgattctccgggagccgttcaatggtgaggttgatgggaagacttccttcccaaagaaaatagatcctccatgcaggatctaggagagtgactgggtgtgccaaaatatgcccagggtcctgccctcagcactagatttaatggggccaagagggtccaaaccccttgctaacataccacttctttgtttaactcctttacctttccagccctttgaggagggaccatgagaacagaaattaccttatgaaaagctacttctgttcctgctttccctctcacgtattgacggtttatttctttgacctcccagagggctgaactctttcaactctgcgctgcccagccttctcagtggacttgcccctcctaagcagagaaggcctatgaggttgcttgctgctgggaagcctggcagagccaattaccaccctctgctgcttagtgcttgggtacctcttgcaataaccagctcttagttgttccctttccctggggcttttccatttaacacatggagcccttcccccagaaggctactttcttgttttagaggaaggtactgcccattgggagatggggacattgggacctcagcaatgaagaacccttgtgaagtaaccaggaggaatggggaaagaagcaagttgggcaggatatggcctacttccataggcttttcttttttcaggtttgatgtaagcatgggcttacatcccccaggtacatacttttacttattgtgggataacctggcactagtaggcaggtaaagtcacaaatttggtgtcttttcaccttttgactgttgacttaatagctcctctcactctgcctggagatacttcctgcctcagatgaggagccagaagaaacagagcccgacttgaatgaactcagctcagagttctaaggaccagcattctgggggccattttctctacaggcaaatggaattgcttttccataacatccaaattgtaatgtggttgctgctgaaggaggaggcagcagcgaggtcctgcggtacccatggggtgatgctacttctgcatgcatctacagggcatctgacacctaacatgagacgtggcatgtgagatgagacttggcatgtgagacatagggtcactagagacccttctgggtcagaggagagagactgaattggactaaacccgtcctctgttcccagcacgtttctcatatagccctcagtcactgagggagtcccccgcaggattggagaggcacattcccttgggacagaggctacaggttggagctttttttcccctgtcccccaaccccatccccacctccacttcagaacatggcaccccacccaactggccaagtgttaagtgatgtgcttattgagagcaactccgggtgtcttttaaaatgtagagaaaaggtgacagtttaaggaaaaatatatatagaataccagaaatgccgtttacccggagaatttttttctccccatttgttttgtttttactcaatgacaccatttttagttttatttcctgatagcaaaaggaaaaaaaacacccatccctcaaaaaggccaaggtcccgtccccctgttgtcggtgatttgtttgtctttctgataggttgaaaattgtgtaataaacttgatgacgctgtcaaaaaaaaaaaaaaaaaaaaaaaaaaaaa

SEQ ID NO: 725 - DNA, 1128 bp, Homo sapiens
gcttctcgttgtgccccgcccgcaagcgccctcctccgggccttcgtgacagccaggtcgtgcgcgggtcatcctgggattggtagttcgctttctctcatttagccagtttctttctctaccggggactccgtgtcccggcatccaccgcggcacctgacccttggcgcttgcgtgttgccctcttccccaccctccctaatttccactccccccaccccacttcgcctgccgcggtcgggtccgcggcctgcgctgtagcggtcgccgccgttccctggaagtagcaacttccctaccccaccccagtcctggtccccgtccagccgctgacgtgaagatgagcagctcagaggaggtgtcctggatttcctggttctgtgggctccgtggcaatgaattcttctgtgaagtggatgaagactacatccaggacaaatttaatcttactggactcaatgagcaggtccctcactaccgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgctgattccctcccccacctgtcctgcagtctttgacttttcctttcttttttgccaccctttcaggaaccctgtatggtttttagtttaaattaaaggagtcgttattgtggtgggaatatgaaataaagtagaagaaaaggcc

SEQ ID NO: 726 - DNA, 886 bp, Homo sapiens
ggggccgcgcgtgctgcagccgccgctgctgctgctcctgctggcgctgctgctggcggcgctgccgtgcggtgccgaagaggcctcgccgctgcgccccgcgcaggtcacgttgtcgccgccgccggccgtgacgaacgggagccagccgggcgcgccacacaacagcacgcacacgcgtccgccgggggcgtcgggctcggcgctgacgcgctccttctacgtgatcctgggcttctgcggcctgaccgcgctctacttcctgatccgggcgtttaggttgaagaagcctcagcggaggcgatacggcctcctcgccaacactgaggaccccacggagatggcctcgctggacagcgacgaggagacggtctttgagtcccggaatctgagatgatgctgagccagggaggcggcccttccagcagccatgagggaaggacaggagatggggcccaccccagtgcccagcaaccccctgctccaccgctcattcccctgctggccccggggctggtctcacccagtgccaacccgagagctccttttggaacctgcacagcccgccgacctgttgccacctgcacccaccgctggaccatgcagcctcgcctcctggatgctgtcccagcctggccgagggtcccaggtgaagactggagggaccccaacagccaccgcccaggacgctgaggctcccttgcctgactgtgacttgtgcctctctcctgcccccgtggggacatggcagcccagagccaaggctgggtgggcaggtgacccaaggaacctttctgggaacaccttctcgccgggctgggaacaataaatgcagccatgtctctgcaaaaaaaaaaaaaaaaaaaaaaaaaaaaaaaaaaaaaaa

SEQ ID NO: 727 - DNA, 2284 bp, Homo sapiens
tggggcggacgcggcggacgtgggtgagggcgcggccgtaagagagcgggacgcggggtgcccggcgcgtggtgggggtccccggcgcctgcccccacggcacccaagaaggcctggccagggtaccctccgcggagcccgggggtggggggcgcgggcccggcgccgcgatgggcccgggacccccagcggccggagcggcgccgtccccgcggccgctgtccctggtggcgcggctgagctacgccgtgggccacttcctcaacgacctgtgcgcgtccatgtggttcacctacctgctgctctacctgcactcggtgcgcgcctacagctcccgcggcgcggggctgctgctgctgctgggccaggtggccgacgggctgtgcacaccgctcgtgggctacgaggccgaccgcgccgccagctgctgcgcccgctacggcccgcgcaaggcctggcacctggtcggcaccgtctgcgtcctgctgtccttccccttcatcttcagcccctgcctgggctgtggggcggccacgcccgagtgggctgccctcctctactacggcccgttcatcgtgatcttccagtttggctgggcctccacacagatctcccacctcagcctcatcccggagctcgtcaccaacgaccatgagaaggtggagctcacggcactcaggtatgcgttcaccgtggtggccaacatcaccgtctacggcgccgcctggctcctgctgcacctgcagggctcgtcgcgggtggagcccacccaagacatcagcatcagcgaccagctggggggccaggacgtgcccgtgttccggaacctgtccctgctggtggtgggtgtcggcgccgtgttctcactgctattccacctgggcacccgggagaggcgccggccgcatgcggaggagccaggcgagcacacccccctgttggcccctgccacggcccagcccctgctgctctggaagcactggctccgggagccggctttctaccaggtgggcatactgtacatgaccaccaggctcatcgtgaacctgtcccagacctacatggccatgtacctcacctactcgctccacctgcccaagaagttcatcgcgaccattcccctggtgatgtacctcagcggcttcttgtcctccttcctcatgaagcccatcaacaagtgcattgggaggaacatgacctacttctcaggcctcctggtgatcctggcctttgccgcctgggtggcgctggcggagggactgggtgtggccgtgtacgcagcggctgtgctgctgggtgctggctgtgccaccatcctcgtcacctcgctggccatgacggccgacctcatcggtccccacacgaacagcggagcgttcgtgtacggctccatgagcttcttggataaggtggccaatgggctggcagtcatggccatccagagcctgcacccttgcccctcagagctctgctgcagggcctgcgtgagcttttaccactgggcgatggtggctgtgacgggcggcgtgggcgtggccgctgccctgtgtctctgtagcctcctgctgtggccgacccgcctgcgacgctcttttcttgcctggagaagagggaggggagaggacaagggccctggctactcctggattcctacagtccttgtccagcctccaagacccacaagtcccttcctctgggaagcccccctggcctggaggtgcaccaggaagaagtggtctggggctggcactaagccatggcccagggaagactgggggacccactaggccaggatgagacctgcacgcagtggctcacagcagcacgatttgtgacagcccgaggcggagaacaccgaacacccagtgaaggtgaggggatcagcacggcgccgccaccgtgctggaacgagactcagccacaaggaggtgcgaagctctgacccaggccacagtgcggatgcaccttgaggatgtcacgctcagtgagagacaccagacacagaagggtacgctgtgatcccacttctatgaaatgtccaggacagaccaatccacagaatcagggagaggattcgtgggtgccgggactggggagggggacctgggggtgactaggtgacataatggggacagggctgccttctgggtgatgagaatgttctggaatcagatgggatggctgcacggcgtggtgaaggtactgaacgccacctcactgtaagacggtagattttgtattttaccacaataaacaaaacaaaacaaaaccaaaccaaacccaa

SEQ ID NO: 728 - DNA, 4357 bp, Homo sapiens
ccccgccccgctctttcgcttcccgggccgccggcagccgccgccagccgcagccatgggccgggcccggccgggccaacgcgggccgcccagccccggccccgccgcgcagcctcccgcgccaccgcgccgccgcgcccgttccctggcgctgctcggagccctgctggccgccgccgctgccgccgccgtccgggtctgcgcccgccacgccgaggcccaggcggccgcgcggcaggaactggcgctgaagaccctggggacagatggcctttttctcttttcctccttggacactgacggggatatgtacatcagccctgaggagttcaaacccattgctgagaagctaacagggtcttgttctgtcacccagactggagtgcagtggtgcagtcacagctcactgcagcctcaacttccctggctcaattgatcctcctgcctcagcctcctgaggtcaactcccgcggccagctgcgaggaggaggagttgccccctgaccctagcgaggagacgctcaccatagaagcccgattccagcctctgctcccggagaccatgaccaagagcaaagatggcttcctaggggtctcccgcctcgccctgtccggcctccgaaactggacagccgccgcctcaccaagtgcagtgtttgccacccgccacttccagcccttccttcccccgccaggccaggagctgggtgagccctggtggatcatccccagtgagctgagcatgttcactggctacctgtccaacaaccgcttctatccaccgccgcccaagggcaaggaggtcatcatccaccggctcctgagcatgttccaccctcggccctttgtgaagacccgctttgcccctcagggagctgtggcctgcctgactgccatcagcgacttctactacactgtgatgttccggatccatgccgagttccagctcagtgagccgcccgacttccccttttggttctcccctgctcagttcaccggccacatcatcctctccaaagacgccacccacgtccgcgacttccggctcttcgtgcccaaccacaggtctctgaatgtggacatggagtggctttacggggccagtgaaagcagcaacatggaggtggacatcggctacataccccagatggagctggaggccacgggcccctctgtgccctccgtgatcctggatgaggatggcagcatgatcgacagccacctgccttcaggggagcccctgcagtttgtgtttgaggagatcaagtggcagcaggagctgagctgggaggaggctgcccggcgcctggaggtggccatgtaccccttcaagaaggtctcctacttgccgttcactgaggccttcgaccgagccaaggctgagaacaagctggtgcactcaatcctgctgtggggggccctggatgaccagtcctgctgaggttcagggcggactctccgggagactgtcctggaaagttcgcccatcctcaccctgctcaacgagagcttcatcagcacctggtccctggtgaaggagctggaggaactgcagaacaaccaggagaactcgtcccaccagaagctggctggcctgcacctggagaagtacagcttccccgtggagatgatgatctgcctgcccaatggcaccgtggtccatcacatcaatgccaactacttcttggacatcacctccgtgaagcccgaggaaatcgagagcaatctcttcagcttctcatccacctttgaagacccgtccacggccacctacatgcagttcctgaaggagggactccggcgtggcctgcccctcctccagccctagagtgcctggacgggatctgatgcacaggcccccacgcctcagagccagagtggtcctcagcccatttcagactgcagatgccgcccactcccaccccactcctaggctgccttggagggtacaagatccactgagggtggccaccacagccttggctccatggtggcgggtagacaagggatgcctgggctgactgggcagaggaacctctagctctgactgtcactcggctctccctacccatttggctctggaagctgcttggcccccccagatcagggcctgggtgaactccctggacctttcctagccagccgcacagtctaggcccttgtggggtgaagaatggagggaggagcaggctaggaagacggggccaccaccctctccttgctttcagcccttcccacaggaaacatcaagaagccccagccaggaggggccaggctgccaaggcggctcccctgtttatctagagccttcgttcctggccataccccggactgccctcctgtgcctgatgtccccagctggggtcagtctcaacaggagccagtcttctggagcctctgggcagaaccctccatcagagtggaaatcagacgggaccccctgcagcttccctgaccacgccactgaccagctatctggggaagtttactgtgaaggggtttctgcctttagcaatggggttcactaagggggttcccgaggcccagggccaaggcactcccaccgcctaccttagcacagggtctctgcaggactgcgggagccagcgctcctgccgcccctcttgcccctcagaccttgcatccacagaagcacaacccagccaaacaccacagccttctccagagccggcactgtcccggcaaccaggggtgccccaggctagctcttctacctctggggcaccacggactccccttggccactcttgggactttggtccacgtcctgagccactgaccacggccagtctctctttttatatgtgcagaaaagtgtttttacacaaactttctcatggtttgtaggtatttttttataaccccagtgctgaggagaaaggaggggcagtggcttccccggcagcagccccatgatggctgaatccgaaatcctcgatgggtccagcttgatgtctttgcagctgcacctatgggaagaagtagtcctctcttccttctcctcttcagctttttaaaaacagtcctcagaggatccatgatccccagcactgtcccatcctccacaaaggcccacaggcatgcctgtactctctttcattaaggtcttgaagtcaggctgccccctccccagcccccagttctctccccaccccctcaccccacccggggctcactcagcctggcagaggaagaaggaaggcagacatctccgcagccactcctgggccttttatgtgccgagttaccccacttgccttgggcgtgtccactgagccttccccagccagtcttgttctcaattttgttttgttttgttttgagacggagtcttgctctgtcacccaggctggagtgctatggctcgatcttggctcactgcaacctccacctcccaggttcaagcaattctcttgcctcagcctcccgagtagctgggattacaggtgcatgccaccatggctggctaatttttgtatttttagtagagatggggtttcaccatattggtcaggctgatctggaacttctgacctcaggtgatccacctgcctcagcctcccaaagtgctgggattacaggcgtgagcaatcgtgcccagccttgttcttaattttgtatcatccagtcatcgctaatattacacgcaccttctcacttaatcctcacgacaagcctgtgaggcagatgctcattgttcccatcttgatgaaacttgagtctcagggaagtgaagtgacttgcccagggtcactcaggtagagttgagattcaaacccacatgtggctccaaagtctgcatctggatttgggggtgttttttggcatggcaccctcacctctctccctgcctgttttccccaaagtggaaaggaaggcctttcaaaccagagtgtctcactcccctctgacctccagaccagatggggcatgagccagccagctcagccaggctccctgtgtcctgggaggaagtgtccccatcccccatgccccttatggggagggagggcgtctgatgctctctctctgcctccccccccatcctgtcaggcacaggtgacgggggcagcccatgcgagcccttctcctgctgctctgggagggccagttccacattgagccagcctggtcccatggaaaatgatggcctgggctttctgaggccttatctgatgcctctgcagttcatgtcccccaccaggcctcgaggctcagggtgggagagggccccgggctgccctgtcactcctctaacacttccctcccctgtccccaacatgccctgtaataaaattagagaagactaacaaaaaaaaaaaaaaaaaaaaaaaaa

SEQ ID NO: 729 - DNA, 2144 bp, Homo sapiens
acagcagcggcgcggagactgcggggcgggccatggcggcgaacctgagccggaacgggccagcgctgcaagaggcctacgtgcgggtggtcaccgagaagtccccgaccgactgggctctctttacctatgaaggcaacagcaatgacatccgcgtggctggcacaggggagggtggcctggaggagatggtggaggagctcaacagcgggaaggtgatgtacgccttctgcagagtgaaggaccccaactctggactgcccaaatttgtcctcatcaactggacaggcgagggcgtgaacgatgtgcggaagggagcctgtgccagccacgtcagcaccatggccagcttcctgaagggggcccatgtgaccatcaacgcacgggccgaggaggatgtggagcctgagtgcatcatggagaaggtggccaaggcttcaggtgccaactacagctttcacaaggagagtggccgcttccaggacgtgggaccccaggccccagtgggctctgtgtaccagaagaccaatgccgtgtctgagattaaaagggttggtaaagacagcttctgggccaaagcagagaaggaggaggagaaccgtcggctggaggaaaagcggcgggccgaggaggcacagcggcagctggagcaggagcgccgggagcgtgagctgcgtgaggctgcacgccgggagcagcgctatcaggagcagggtggcgaggccagcccccagaggacgtgggagcagcagcaagaagtggtttcaaggaaccgaaatgagcaggagtctgccgtgcacccgagggagattttcaagcagaaggagagggccatgtccaccacctccatctccagtcctcagcctggcaagctgaggagccccttcctgcagaagcagctcacccaaccagagacccactttggcagagagccagctgctgccatctcaaggcccagggcagatctccctgctgaggagccggcgcccagcactcctccatgtctggtgcaggcagaagaggaggctgtgtatgaggaacctccagagcaggagaccttctacgagcagcccccactggtgcagcagcaaggtgctggctctgagcacattgaccaccacattcagggccaggggctcagtgggcaagggctctgtgcccgtgccctgtacgactaccaggcagccgacgacacagagatctcctttgaccccgagaacctcatcacgggcatcgaggtgatcgacgaaggctggtggcgtggctatgggccggatggccattttggcatgttccctgccaactacgtggagctcattgagtgaggctgagggcacatcttgcccttcccctctcagacatggcttccttattgctggaagaggaggcctgggagttgacattcagcactcttccaggaataggacccccagtgaggatgaggcctcagggctccctccggcttggcagactcagcctgtcaccccaaatgcagcaatggcctggtgattcccacacatccttcctgcatcccccgaccctcccagacagcttggctcttgcccctgacaggatactgagccaagccctgcctgtggccaagccctgagtggccactgccaagctgcggggaagggtcctgagcaggggcatctgggaggctctggctgccttctgcatttatttgccttttttctttttctcttgcttctaaggggtggtggccaccactgtttagaatgacccttgggaacagtgaacgtagagaattgtttttagcagagtttgtgaccaaagtcagagtggatcatggtggtttggcagcagggaatttgtcttgttggagcctgctctgtgctccccactccatttctctgtccctctgcctgggctatgggaagtggggatgcagatggccaagctcccaccctgggtattcaaaaacggcagacacaacatgttcctccacgcggctcactcgatgcctgcaggccccagtgtgtgcctcaactgattctgacttcaggaaaagtaacacagagtggccttggcctgttgtcttcccctattttctgtcccagctcatccgtgtctctgaagaacaaatatgcttttggaccacgaaaaaaaaaaaaaaaaaaaaa

SEQ ID NO: 730 - DNA, 990 bp, Homo sapiens
tccggagcccggctcgctggggcagcatggcggggtcgccgctgctctgggggccgcgggccgggggcgtcggccttttggtgctgctgctgctcggcctgtttcggccgccccccgcgctctgcgcgcggccggtaaaggagccccgcggcctaagcgcagcgtctccgcccttggctgagactggcgctcctcgccgcttccggcggtcagtgccccgaggtgaggcggcgggggcggtgcaggagctggcgcgggcgctggcgcatctgctggaggccgaacgtcaggagcgggcgcgggccgaggcgcaggaggctgaggatcagcaggcgcgcgtcctggcgcagctgctgcgcgtctggggcgccccccgcaactctgatccggctctgggcctggacgacgaccccgacgcgcctgcagcgcagctcgctcgcgctctgctccgcgcccgccttgaccctgccgccctcgcagcccagcttgtccccgcgcccgtccccgccgcggcgctccgaccccggcccccggtctacgacgacggccccgcgggcccggatgctgaggaggcaggcgacgagacacccgacgtggaccccgagctgttgaggtacttgctgggacggattcttgcgggaagcgcggactccgagggggtggcagccccgcgccgcctccgccgtgccgccgaccacgatgtgggctctgagctgccccctgagggcgtgctgggggcgctgctgcgtgtgaaacgcctagagaccccggcgccccaggtgcctgcacgccgcctcttgccaccctgagcactgcccggatcccgtgcaccctgggacccagaagtgcccccgccatcccgccaccaggactgctccccgccagcacgtccagagcaacttaccccggccagccagccctctcacccgaggatccctaccccctggccccacaataaacatgatctgaagcaaaaaaaaaaaaaaaaaa

SEQ ID NO: 731 - DNA, 8187 bp, Homo sapiens
cccgagaagcggcggggcggcgggccggcgggcggggcgcagagccaggcagcgcaggtatagccaggctggagaaaagaagctgccaccatggttgcactttcactgaagatcagcattgggaatgtggtgaagacgatgcagtttgagccgtctaccatggtgtacgacgcctgccgcatcattcgtgagcggatcccagaggccccagctggtcctcccagcgactttgggctctttctgtcagatgatgaccccaaaaagggtatatggctggaggctgggaaagctttggactactacatgctccgaaatggggacactatggagtacaggaagaaacagagacccctgaagatccgtatgctggatggaactgtgaagacgatcatggtggatgactctaagactgtcactgacatgctcatgaccatctgtgcccgcattggcatcaccaatcatgatgaatattcattggttcgagagctgatggaagagaaaaaggaggaaataacagggaccttaagaaaggacaagacattgctgcgagatgaaaagaagatggagaaactaaagcagaaattgcacacagatgatgagttgaactggctggaccatggtcggacactgagggagcagggtgtagaggagcacgagacgctgctgctgcggaggaagttcttttactcagaccagaatgtggattcccgggaccctgtacagctgaacctcctgtatgtgcaggcacgagatgacatcctgaatggctcccaccctgtctcctttgacaaggcctgtgagtttgctggcttccaatgccagatccagtttgggccccacaatgagcagaagcacaaggctggcttccttgacctgaaggacttcctgcccaaggagtatgtgaagcagaagggagagcgtaagatcttccaggcacacaagaattgtgggcagatgagtgagattgaggccaaggtccgctacgtgaagctagcccgttctctcaagacttacggtgtctccttcttcctggtgaaggaaaaaatgaaagggaagaacaagctagtgcccaggcttctgggcatcaccaaggagtgtgtgatgcgagtggatgagaagaccaaggaagtgatccaggagtggaacctcaccaacatcaaacgctgggctgcgtctcccaaaagcttcaccctggattttggagattaccaagatggctattactcagtacagacaactgaaggggagcagattgcacagctcattgccggctacatcgatatcatcctgaagaagaaaaaaagcaaggatcactttgggctggaaggagatgaggagtctactatgctggaggactcagtgtcccccaaaaagtcaacagtcctgcagcagcaatacaaccgggtggggaaagtggagcatggctctgtggccctgcctgccatcatgcgctctggagcctctggtcctgagaatttccaggtgggcagcatgccccctgcccagcagcagattaccagcggccagatgcaccgaggacacatgcctcctctgacttcagcccagcaggcactcactggaaccattaactccagcatgcaggccgtgcaggctgcccaggccaccctggatgactttgacactctgccgcctcttggccaggatgctgcctctaaggcctggcgtaaaaacaagatggatgaatcaaagcatgagatccactctcaggtagatgccatcacagctggtactgcgtctgtggtgaacctgacagcaggggaccctgctgagacagactataccgcagtgggctgtgcagtcaccacaatctcctccaacctgacggagatgtcccgtggggtgaagctgctggctgccttgctggaggacgaaggcggcagtggtcggcccctgttgcaggcagcaaagggccttgcgggagcagtgtcagaactgctgcgcagtgcccaaccagccagtgctgagccccgtcagaacctgctgcaagcagctgggaacgtgggccaggccagtggggagctgttgcaacaaattggggaaagtgatactgacccccacttccaggatgcgctaatgcagctcgccaaagctgtggcaagtgctgcagctgccctggtcctcaaggccaagagtgtggcccagcggacagaggactcgggacttcagacccaagttattgctgcagcaacacagtgtgccctatccacttcccaactagtggcctgtactaaggtggtggcacctacaatcagctcacctgtctgccaagagcaactggtggaggctggacgactggtagccaaagccgtggagggctgtgtgtctgcctcccaggcagctacagaggatgggcaactgttgcgaggggtaggagcagcagccacagctgtcacccaggccctaaatgagctgctgcagcatgtgaaagcccatgccacaggggctgggcctgctggccgttatgaccaggctactgacaccatcctaaccgtcactgagaacatctttagctccatgggtgatgctggggagatggtgggacaggcccgcatcctggcccaagccacatctgacctggtcaatgccatcaaggctgatgctgagggggaaagtgatctggagaactcccgcaagctcttaagtgctgccaagatcctagctgatgccacagccaagatggtagaggctgccaagggagcagctgcccaccctgacagtgaggagcagcagcagcggctgcgggaggcagctgaggggctgcgcatggccaccaatgcagctgcgcagaatgccatcaagaaaaagctggtgcagcgcctggagcatgcagccaagcaggctgcagcctcagccacacagaccatcgctgcagctcagcacgcagcctctacccccaaagcctctgccggcccccagcccctgctggtgcagagctgcaaggcagtggcagagcagattccactgctggtgcagggcgtccgaggaagccaagcccagcctgacagccccagcgctcagcttgccctcattgctgccagccagagcttcctgcagccaggtgggaagatggtggcagctgcaaaggcctcagtgccaacgattcaggaccaggcttcagccatgcagctgagtcagtgtgccaagaacctgggcaccgcgctggctgaactccggacggctgcccagaaggctcaggaagcatgtggacctttggagatggattctgcactgagtgtggtacagaatctagagaaagatctacaggaagtgaaggcagcagctcgagatggcaagcttaaacccttacctggggagacaatggagaagtgtacccaggacctgggcaacagcaccaaagccgtgagctcagccatcgcccagctactgggagaggttgcccagggcaatgagaattatgcaggtattgcagctcgggatgtggcaggtgggctgcggtcactggcccaggccgctaggggagtcgctgcactgacgtcagatcctgcagtgcaggccattgtacttgatacggccagtgatgtgctggacaaggccagcagcctcattgaggaggcgaaaaaggcagctggccatccaggggaccctgagagccagcagcggcttgcccaggtggctaaagcagtgacccaggctctgaaccgctgtgtcagctgcctacctggccagcgcgatgtggataatgccctgagggcagttggagatgccagcaagcgactcctgagtgactcgcttcctcctagcactgggacatttcaagaagctcagagccggttgaatgaagctgctgctgggctgaatcaggcagccacagaactggtgcaggcctctcggggaacccctcaggacctggctcgagcctcaggccgatttggacaggacttcagcaccttcctggaagctggtgtggagatggcaggccaggctccgagccaggaggaccgagcccaagttgtgtccaacttgaagggcatctccatgtcttcaagcaaacttcttctggctgccaaggccctgtccacggaccctgctgcccctaacctcaagagtcagctggctgcagctgccagggcagtaactgacagcatcaatcagctcatcactatgtgcacccagcaggcacccggccagaaggagtgtgataacgccctgcgggaattggagacggtccgggaactcctggagaacccagtccagcccatcaatgacatgtcctactttggttgcctggacagtgtaatggagaactcaaaggtgctgggcgaggccatgactggcatctcccaaaatgccaagaacggaaacctgccagagtttggagatgccatttccacagcctcaaaggcactttgtggcttcaccgaggcagctgcacaggctgcatatctggttggtgtctctgaccccaatagccaagctggacagcaagggctagtggagcccacacagtttgcccgtgcaaaccaggcaattcagatggcctgccagagtttgggagagcctggctgtacccaggcccaggtgctctctgcagccaccattgtggctaaacacacctctgcactgtgtaacagctgtcgcctggcttctgcccgtaccaccaatcctactgccaagcgccagtttgtacagtcagccaaggaggtggccaacagcacagctaatcttgtcaagaccatcaaggcgctagatggggccttcacagaggagaaccgtgcccagtgccgagcagcaacagcccctctgctggaggctgtggacaatctgagtgcctttgcgtccaaccctgagttctccagcattcctgcccagatcagccctgagggtcgggctgccatggagcccattgtgatctctgccaagacaatgttagagagtgccgggggactcatccagacagcccgggccctcgcagtcaatccccgggaccccccgagctggtcggtgctggccggccactcccgtactgtctcagactccatcaagaagctaattacaagcatgagggacaaggctccagggcagctggagtgtgaaacggccattgcagctctgaacagttgtctacgggacctagaccaggcttccctcgctgcagtcagccagcagcttgctccccgtgagggaatctctcaagaggccttgcacactcagatgctcactgcagtccaagagatctcccatctcattgagccgctggccaatgctgcccgggctgaagcctcccagctgggacacaaggtgtcccagatggcgcagtactttgagccgctcaccctggctgcagtgggtgctgcctccaagaccctgagccacccgcagcagatggcactcctggaccagactaaaacattggcagagtctgccctgcagttgctatacactgccaaggaggctggtggtaacccaaagcaagcagctcacacccaggaagccctggaggaggctgtgcagatgatgaccgaggccgtagaggacctgacaacaaccctcaacgaggcagccagtgctgctggggtcgtgggtggcatggtggactccatcacccaggccatcaaccagctagatgaaggaccaatgggtgaaccagaaggttccttcgtggattaccaaacaactatggtgcggacagccaaggccattgcagtgaccgttcaggagatggttaccaagtcaaacaccagcccagaggagctgggccctcttgctaaccagctgaccagtgactatggccgtctggcctcggaggccaagcctgcagcggtggctgctgaaaatgaagagataggttcccatatcaaacaccgggtacaggagctgggccatggctgtgccgctctggtcaccaaggcaggcgccctgcagtgcagccccagtgatgcctacaccaagaaggagctcatagagtgtgcccggagagtctctgagaaggtctcccacgtcctggctgcgctccaggctgggaatcgtggcacccaggcctgcatcacagcagccagcgctgtgtctggtatcattgctgacctcgacaccaccatcatgttcgccactgctggcacgctcaatcgtgagggtactgaaactttcgctgaccaccgggagggcatcctgaagactgcgaaggtgctggtggaggacaccaaggtcctggtgcaaaacgcagctgggagccaggagaagttggcgcaggctgcccagtcctccgtggcgaccatcacccgcctcgctgatgtggtcaagctgggtgcagccagcctgggagctgaggaccctgagacccaggtggtactaatcaacgcagtgaaagatgtagccaaagccctgggagacctcatcagtgcaacgaaggctgcagctggcaaagttggagatgaccctgctgtgtggcagctaaagaactctgccaaggtgatggtgaccaatgtgacatcattgcttaagacagtaaaagccgtggaagatgaggccaccaaaggcactcgggccctggaggcaaccacagaacacatacggcaggagctggcggttttctgttccccagagccacctgccaagacctctaccccagaagacttcatccgaatgaccaagggtatcaccatggcaaccgccaaggccgttgctgctggcaattcctgtcgccaggaagatgtcattgccacagccaatctgagccgccgtgctattgcagatatgcttcgggcttgcaaggaagcagcttaccacccagaagtggcccctgatgtgcggcttcgagccctgcactatggccgggagtgtgccaatggctacctggaactgctggaccatgtactgctgaccctgcagaagccaagcccagaactgaagcagcagttgacaggacattcaaagcgtgtggctggttccgtcactgagctcatccaggctgctgaagccatgaagggaacagaatgggtagacccagaggaccccacagtcattgctgagaatgagctcctgggagctgcagccgccattgaggctgcagccaaaaagctagagcagctgaagccccgggccaaacccaaggaggcagatgagtccttgaactttgaggagcagatactagaagctgccaagtccattgcagcagccaccagtgcactggtaaaggctgcgtcggctgcccagagagaactagtggcccaagggaaggtgggtgccattccagccaatgcactggacgatgggcagtggtcccagggcctcatttctgctgcccggatggtggctgcggccaccaacaatctgtgtgaggcagccaatgcagctgtacaaggccatgccagccaggagaagctcatctcatcagccaagcaggtagctgcctccacagcccagctccttgtggcctgcaaggtcaaggctgaccaggactcggaggcaatgaaacgacttcaggctgctggcaacgcagtgaagcgagcctcagataatctggtgaaagcagcacagaaggctgcagcctttgaagagcaggagaatgagacagtggtggtgaaagagaagatggttggcggcattgcccagatcatcgcagcacaggaagaaatgcttcggaaggaacgagagctggaagaggcgcggaagaaactggcccagatccggcagcagcagtacaagtttctgccttcagagcttcgagatgagcactaaagaagcctcttctatttaatgcagacccggcccagagactgtgcgtgccactaccaaagccttctgggctgtcggggcccaacctgcccaaccccagcactccccaaagtgcctgccaaaccccagggcctggccccgcccagtcccgcagtacatcccctgtcccctccccaaccccaagtgccttcatgccctagggccccccaagtgcctgcccctccccagagtattaacgctccaagagtattattaacgctgctgtacctcgatctgaatctgccggggccccagcccactccaccctgccagcagcttccagccagtccccacagcctcatcagctctcttcaccgttttttgatactatcttcccccacccccagctacccataggggctgcagagttataagccccaaacaggtcatgctccaataaaaatgattctacctacaaaaaaaaaaaaaaaaaaa

SEQ ID NO: 732 - DNA, 889 bp, Homo sapiens
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgcaaaaaaaaaaaaaaaaaaa

SEQ ID NO: 733 - DNA, 22 bp, Homo sapiens
agctgcttttgggattccgttg

SEQ ID NO: 734 - DNA, 21 bp, Homo sapiens
ctaccatagggtaaaaccact

SEQ ID NO: 735 - DNA, 22 bp, Homo sapiens
ccacacacttccttacattcca

SEQ ID NO: 736 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 737 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 738 - RNA, 86 nt, Homo sapiens
ugcuucccgaggccacaugcuucuuuauauccccauauggauuacuuugcuauggaauguaaggaagugugugguuucggcaagug

SEQ ID NO: 739 - DNA, 25 bp, Homo sapiens
gaatgaactcgagttgactggaatg

SEQ ID NO: 740 - DNA, 25 bp, Homo sapiens
aatgaactcgagttgactggaatgg

SEQ ID NO: 741 - DNA, 25 bp, Homo sapiens
atgaactcgagttgactggaatgga

SEQ ID NO: 742 - DNA, 25 bp, Homo sapiens
tgaactcgagttgactggaatggaa

SEQ ID NO: 743 - DNA, 25 bp, Homo sapiens
gaactcgagttgactggaatggaat

SEQ ID NO: 744 - DNA, 25 bp, Homo sapiens
aactcgagttgactggaatggaatg

SEQ ID NO: 745 - DNA, 25 bp, Homo sapiens
gaatggaatcaactcgagtggaatg

SEQ ID NO: 746 - DNA, 25 bp, Homo sapiens
aatggaatcaactcgagtggaatgg

SEQ ID NO: 747 - DNA, 25 bp, Homo sapiens
atggaatcaactcgagtggaatgga

SEQ ID NO: 748 - DNA, 25 bp, Homo sapiens
tggaatcaactcgagtggaatggaa

SEQ ID NO: 749 - DNA, 25 bp, Homo sapiens
ggaatcaactcgagtggaatggaat

SEQ ID NO: 750 - DNA, 25 bp, Homo sapiens
gaatcaactcgagtggaatggaatg

SEQ ID NO: 751 - DNA, 25 bp, Homo sapiens
aatcaactcgagtggaatggaatgg

SEQ ID NO: 752 - DNA, 25 bp, Homo sapiens
atcaactcgagtggaatggaatgga

SEQ ID NO: 753 - DNA, 25 bp, Homo sapiens
caactcgagtggaatggaatggaat

SEQ ID NO: 754 - DNA, 25 bp, Homo sapiens
aatgcagtacaatgcaatagaatgg

SEQ ID NO: 755 - DNA, 25 bp, Homo sapiens
taagcaagagccatggcatggtgaa

SEQ ID NO: 756 - DNA, 25 bp, Homo sapiens
gcaagagccatggcatggtgaaaat

SEQ ID NO: 757 - DNA, 25 bp, Homo sapiens
agagtctggccaatctacaaataga

SEQ ID NO: 758 - DNA, 25 bp, Homo sapiens
tggccaatctacaaatagagaacaa

SEQ ID NO: 759 - DNA, 25 bp, Homo sapiens
aaacggcagacaccaacatggatct

SEQ ID NO: 760 - DNA, 25 bp, Homo sapiens
cggcagacaccaacatggatctcat

SEQ ID NO: 761 - DNA, 25 bp, Homo sapiens
acatggatctcatgggggattggat

SEQ ID NO: 762 - DNA, 25 bp, Homo sapiens
atctcatgggggattggatattgta

SEQ ID NO: 763 - DNA, 25 bp, Homo sapiens
agatgacagtgatcgtcatttggca

SEQ ID NO: 764 - DNA, 25 bp, Homo sapiens
tgacagtgatcgtcatttggcacaa

SEQ ID NO: 765 - DNA, 25 bp, Homo sapiens
tgatcgtcatttggcacaacatctt

SEQ ID NO: 766 - DNA, 25 bp, Homo sapiens
ggcacaacatcttaacaacgaccga

SEQ ID NO: 767 - DNA, 25 bp, Homo sapiens
cccattatttacataaacctaccat

SEQ ID NO: 768 - DNA, 25 bp, Homo sapiens
aacctaccattcggtaaccatgtga

SEQ ID NO: 769 - DNA, 25 bp, Homo sapiens
tcagttgacctcagtgaattctgtg

SEQ ID NO: 770 - DNA, 25 bp, Homo sapiens
gagatgcagactcccgtgtagtttc

SEQ ID NO: 771 - DNA, 25 bp, Homo sapiens
actcccgtgtagtttcagattcttg

SEQ ID NO: 772 - DNA, 25 bp, Homo sapiens
aattaggctttcctaacctgaagcg

SEQ ID NO: 773 - DNA, 25 bp, Homo sapiens
taggctttcctaacctgaagcgcct

SEQ ID NO: 774 - DNA, 25 bp, Homo sapiens
gctttcctaacctgaagcgccttca

SEQ ID NO: 775 - DNA, 25 bp, Homo sapiens
ccagtgtgagacccgaaccatgctg

SEQ ID NO: 776 - DNA, 25 bp, Homo sapiens
tgagacccgaaccatgctgctgcag

SEQ ID NO: 777 - DNA, 25 bp, Homo sapiens
cctcggctcctacagctaccggagt

SEQ ID NO: 778 - DNA, 25 bp, Homo sapiens
cagctaccggagtccccactggggc

SEQ ID NO: 779 - DNA, 25 bp, Homo sapiens
ctggggcagcacctactccgtgtca

SEQ ID NO: 780 - DNA, 25 bp, Homo sapiens
ctactccgtgtcagtggtggagacc

SEQ ID NO: 781 - DNA, 25 bp, Homo sapiens
cgtgtcagtggtggagaccgactac

SEQ ID NO: 782 - DNA, 25 bp, Homo sapiens
cgaccagtacgcgctgctgtacagc

SEQ ID NO: 783 - DNA, 25 bp, Homo sapiens
gtacagccagggcagcaagggccct

SEQ ID NO: 784 - DNA, 25 bp, Homo sapiens
tggcgaggacttccgcatggccacc

SEQ ID NO: 785 - DNA, 25 bp, Homo sapiens
caaggcccagggcttcacagaggat

SEQ ID NO: 786 - DNA, 25 bp, Homo sapiens
ccagggcttcacagaggataccatt

SEQ ID NO: 787 - DNA, 25 bp, Homo sapiens
ccaaaccgataagtgcatgacggaa

SEQ ID NO: 788 - DNA, 25 bp, Homo sapiens
gtgcatgacggaacaataggactcc

SEQ ID NO: 789 - DNA, 25 bp, Homo sapiens
acaataggactccccagggctgaag

SEQ ID NO: 790 - DNA, 25 bp, Homo sapiens
ggactccccagggctgaagctggga

SEQ ID NO: 791 - DNA, 25 bp, Homo sapiens
gctgggatcccggccagccaggtga

SEQ ID NO: 792 - DNA, 25 bp, Homo sapiens
tggatgtctctgctctgttccttcc

SEQ ID NO: 793 - DNA, 25 bp, Homo sapiens
actcgggcttcatcctgcacaataa

SEQ ID NO: 794 - DNA, 25 bp, Homo sapiens
acaataaactccggaagcaagtcag

SEQ ID NO: 795 - DNA, 25 bp, Homo sapiens
ttgcgctgctgtgcctcgatggcaa

SEQ ID NO: 796 - DNA, 25 bp, Homo sapiens
gcctcgatggcaaacggaagcctgt

SEQ ID NO: 797 - DNA, 25 bp, Homo sapiens
aacggaagcctgtgactgaggctag

SEQ ID NO: 798 - DNA, 25 bp, Homo sapiens
agcctgtgactgaggctagaagctg

SEQ ID NO: 799 - DNA, 25 bp, Homo sapiens
aggctagaagctgccatcttgccat

SEQ ID NO: 800 - DNA, 25 bp, Homo sapiens
gaagctgccatcttgccatggcccc

SEQ ID NO: 801 - DNA, 25 bp, Homo sapiens
cgaatcatgccgtggtgtctcggat

SEQ ID NO: 802 - DNA, 25 bp, Homo sapiens
atgccgtggtgtctcggatggataa

SEQ ID NO: 803 - DNA, 25 bp, Homo sapiens
aacgcctgaaacaggtgctgctcca

SEQ ID NO: 804 - DNA, 25 bp, Homo sapiens
aggtgctgctccaccaacaggctaa

SEQ ID NO: 805 - DNA, 25 bp, Homo sapiens
acaagttttgcttattccagtctga

SEQ ID NO: 806 - DNA, 25 bp, Homo sapiens
ttctgttcaatgacaacactgagtg

SEQ ID NO: 807 - DNA, 25 bp, Homo sapiens
acaacactgagtgtctggccagact

SEQ ID NO: 808 - DNA, 25 bp, Homo sapiens
gtctggccagactccatggcaaaac

SEQ ID NO: 809 - DNA, 25 bp, Homo sapiens
ccagactccatggcaaaacaacata

SEQ ID NO: 810 - DNA, 25 bp, Homo sapiens
atgtcgcaggcattactaatctgaa

SEQ ID NO: 811 - DNA, 25 bp, Homo sapiens
gctccccaagaaagcctcagccatt

SEQ ID NO: 812 - DNA, 25 bp, Homo sapiens
caagaaagcctcagccattcactgc

SEQ ID NO: 813 - DNA, 25 bp, Homo sapiens
gggattgcccatccatctgcttaca

SEQ ID NO: 814 - DNA, 25 bp, Homo sapiens
ctgctgtcgtcttagcaagaagtaa

SEQ ID NO: 815 - DNA, 25 bp, Homo sapiens
aggaaatggctcgtcaccttcgtga

SEQ ID NO: 816 - DNA, 25 bp, Homo sapiens
ttcctccctgaacctgagggaaact

SEQ ID NO: 817 - DNA, 25 bp, Homo sapiens
aaactaatctggattcactccctct

SEQ ID NO: 818 - DNA, 25 bp, Homo sapiens
aatctggattcactccctctggttg

SEQ ID NO: 819 - DNA, 25 bp, Homo sapiens
ggattcactccctctggttgatacc

SEQ ID NO: 820 - DNA, 25 bp, Homo sapiens
ctccctctggttgatacccactcaa

SEQ ID NO: 821 - DNA, 25 bp, Homo sapiens
ctggttgatacccactcaaaaagga

SEQ ID NO: 822 - DNA, 25 bp, Homo sapiens
tatcaacgaaacttctcagcatcac

SEQ ID NO: 823 - DNA, 25 bp, Homo sapiens
acgaaacttctcagcatcacgatga

SEQ ID NO: 824 - DNA, 25 bp, Homo sapiens
cagcatcacgatgaccttgaataaa

SEQ ID NO: 825 - DNA, 25 bp, Homo sapiens
taaagaaacagctttcaagtgcctt

SEQ ID NO: 826 - DNA, 25 bp, Homo sapiens
gtgcctttctgcagtttttcaggag

SEQ ID NO: 827 - DNA, 25 bp, Homo sapiens
tttctgcagtttttcaggagcgcaa

SEQ ID NO: 828 - DNA, 25 bp, Homo sapiens
taagctctagttcttaacaaccgac

SEQ ID NO: 829 - DNA, 25 bp, Homo sapiens
tctagttcttaacaaccgacactcc

SEQ ID NO: 830 - DNA, 25 bp, Homo sapiens
tcttaacaaccgacactcctacaag

SEQ ID NO: 831 - DNA, 25 bp, Homo sapiens
cgacactcctacaagatttagaaaa

SEQ ID NO: 832 - DNA, 25 bp, Homo sapiens
tacaacataatctagtttacagaaa

SEQ ID NO: 833 - DNA, 25 bp, Homo sapiens
gctatccagcattcaggtttactca

SEQ ID NO: 834 - DNA, 25 bp, Homo sapiens
atcctgaagctgacagcattcgggc

SEQ ID NO: 835 - DNA, 25 bp, Homo sapiens
cctgaagctgacagcattcgggccg

SEQ ID NO: 836 - DNA, 25 bp, Homo sapiens
aagctgacagcattcgggccgagat

SEQ ID NO: 837 - DNA, 25 bp, Homo sapiens
gctgacagcattcgggccgagatgt

SEQ ID NO: 838 - DNA, 25 bp, Homo sapiens
tgacagcattcgggccgagatgtct

SEQ ID NO: 839 - DNA, 25 bp, Homo sapiens
cattcgggccgagatgtctcgctcc

SEQ ID NO: 840 - DNA, 25 bp, Homo sapiens
ggccgagatgtctcgctccgtggcc

SEQ ID NO: 841 - DNA, 25 bp, Homo sapiens
ggaggtttgaagatgccgcaggatc

SEQ ID NO: 842 - DNA, 25 bp, Homo sapiens
gagatgtctcgctccgtggccttag

SEQ ID NO: 843 - DNA, 25 bp, Homo sapiens
gatgtctcgctccgtggccttagct

SEQ ID NO: 844 - DNA, 25 bp, Homo sapiens
tgtctcgctccgtggccttagctgt

SEQ ID NO: 845 - DNA, 25 bp, Homo sapiens
cgtggccttagctgtgctcgcgcta

SEQ ID NO: 846 - DNA, 25 bp, Homo sapiens
cttagctgtgctcgcgctactctct

SEQ ID NO: 847 - DNA, 25 bp, Homo sapiens
tagctgtgctcgcgctactctctct

SEQ ID NO: 848 - DNA, 25 bp, Homo sapiens
gctgtgctcgcgctactctctcttt

SEQ ID NO: 849 - DNA, 25 bp, Homo sapiens
tgtgctcgcgctactctctctttct

SEQ ID NO: 850 - DNA, 25 bp, Homo sapiens
gcctggaggctatccagcattcagg

SEQ ID NO: 851 - DNA, 25 bp, Homo sapiens
ctggaggctatccagcattcaggtt

SEQ ID NO: 852 - DNA, 25 bp, Homo sapiens
ggaggctatccagcattcaggttta

SEQ ID NO: 853 - DNA, 25 bp, Homo sapiens
gcctggtactcaagcccgcggggac

SEQ ID NO: 854 - DNA, 25 bp, Homo sapiens
ctggtactcaagcccgcggggacat

SEQ ID NO: 855 - DNA, 25 bp, Homo sapiens
tggtactcaagcccgcggggacatt

SEQ ID NO: 856 - DNA, 25 bp, Homo sapiens
ggtactcaagcccgcggggacattg

SEQ ID NO: 857 - DNA, 25 bp, Homo sapiens
gtactcaagcccgcggggacattgg

SEQ ID NO: 858 - DNA, 25 bp, Homo sapiens
actcaagcccgcggggacattggga

SEQ ID NO: 859 - DNA, 25 bp, Homo sapiens
ctcaagcccgcggggacattgggaa

SEQ ID NO: 860 - DNA, 25 bp, Homo sapiens
tcaagcccgcggggacattgggaag

SEQ ID NO: 861 - DNA, 25 bp, Homo sapiens
cctctctgcaccgtactgtggaaaa

SEQ ID NO: 862 - DNA, 25 bp, Homo sapiens
ctctctgcaccgtactgtggaaaag

SEQ ID NO: 863 - DNA, 25 bp, Homo sapiens
tctctgcaccgtactgtggaaaaga

SEQ ID NO: 864 - DNA, 25 bp, Homo sapiens
ctctgcaccgtactgtggaaaagaa

SEQ ID NO: 865 - DNA, 25 bp, Homo sapiens
gaaacacgcacttagtctctaaaga

SEQ ID NO: 866 - DNA, 25 bp, Homo sapiens
aacacgcacttagtctctaaagagt

SEQ ID NO: 867 - DNA, 25 bp, Homo sapiens
acacgcacttagtctctaaagagtt

SEQ ID NO: 868 - DNA, 25 bp, Homo sapiens
acgcacttagtctctaaagagttta

SEQ ID NO: 869 - DNA, 25 bp, Homo sapiens
cgcacttagtctctaaagagtttat

SEQ ID NO: 870 - DNA, 25 bp, Homo sapiens
gcacttagtctctaaagagtttatt

SEQ ID NO: 871 - DNA, 25 bp, Homo sapiens
cacttagtctctaaagagtttattt

SEQ ID NO: 872 - DNA, 25 bp, Homo sapiens
taagacgtgtttgtgtttgtgtgtg

SEQ ID NO: 873 - DNA, 25 bp, Homo sapiens
tgagaagatcccaacaacctttgag

SEQ ID NO: 874 - DNA, 25 bp, Homo sapiens
gatcccaacaacctttgagaatgga

SEQ ID NO: 875 - DNA, 25 bp, Homo sapiens
ctttgagaatggacgctgcatccag

SEQ ID NO: 876 - DNA, 25 bp, Homo sapiens
acgctgcatccaggccaactactca

SEQ ID NO: 877 - DNA, 25 bp, Homo sapiens
catccaggccaactactcactaatg

SEQ ID NO: 878 - DNA, 25 bp, Homo sapiens
ttcctggtttatgccatcggcaccg

SEQ ID NO: 879 - DNA, 25 bp, Homo sapiens
gtttatgccatcggcaccgtactgg

SEQ ID NO: 880 - DNA, 25 bp, Homo sapiens
gatcctggccaccgactatgagaac

SEQ ID NO: 881 - DNA, 25 bp, Homo sapiens
ggccaccgactatgagaactatgcc

SEQ ID NO: 882 - DNA, 25 bp, Homo sapiens
tgagaactatgccctcgtgtattcc

SEQ ID NO: 883 - DNA, 25 bp, Homo sapiens
cctcgtgtattcctgtacctgcatc

SEQ ID NO: 884 - DNA, 25 bp, Homo sapiens
gtattcctgtacctgcatcatccaa

SEQ ID NO: 885 - DNA, 25 bp, Homo sapiens
ctgtacctgcatcatccaacttttt

SEQ ID NO: 886 - DNA, 25 bp, Homo sapiens
ctgcatcatccaactttttcacgtg

SEQ ID NO: 887 - DNA, 25 bp, Homo sapiens
tgcttggatcttggcaagaaaccct

SEQ ID NO: 888 - DNA, 25 bp, Homo sapiens
cacagaccaggtgaactgccccaag

SEQ ID NO: 889 - DNA, 25 bp, Homo sapiens
ccaggtgaactgccccaagctctcg

SEQ ID NO: 890 - DNA, 25 bp, Homo sapiens
aggttctacagggaggctgcaccca

SEQ ID NO: 891 - DNA, 25 bp, Homo sapiens
actccatgttacttctgcttcgctt

SEQ ID NO: 892 - DNA, 25 bp, Homo sapiens
cctgttaccttgctagctgcaaaat

SEQ ID NO: 893 - DNA, 1649 bp, Homo sapiens
agacaaggttttccaagcaagatgaagcccaacatcatctttgtactttccctgctcctcatcttggagaagcaagcagctgtgatgggacaaaaaggtggatcaaaaggccgattaccaagtgaattttcccaatttccacacggacaaaagggccagcactattctggacaaaaaggcaagcaacaaactgaatccaaaggcagtttttctattcaatacacatatcatgtagatgccaatgatcatgaccagtcccgaaaaagtcagcaatatgatttgaatgccctacataagacgacaaaatcacaacgacatctaggtggaagtcaacaactgctccataataaacaagaaggcagagaccatgataaatcaaaaggtcattttcacagggtagttatacaccataaaggaggcaaagctcatcgtgggacacaaaatccttctcaagatcaggggaatagcccatctggaaagggaatatccagtcaatattcaaacacagaagaaaggctgtgggttcatggactaagtaaagaacaaacttccgtctctggtgcacaaaaaggtagaaaacaaggcggatcccaaagcagttatgttctccaaactgaagagctagtagctaacaaacaacaacgtgagactaaaaattctcatcaaaataaagggcattaccaaaatgtggttgaagtgagagaggaacattcaagtaaagtacaaacctcactctgtcctgcgcaccaagacaaactccaacatggatccaaagacattttttctacccaagatgagctcctagtatataacaagaatcaacaccagacaaaaaatctcaatcaagatcaacagcatggccgaaaggcaaataaaatatcataccaatcttcaagtacagaagaaagacgactccactatggagaaaatggtgtgcagaaagatgtatcccaaagcagtatttatagccaaactgaagagaaagcacagggcaagtctcaaaaacagataacaattcccagtcaagagcaagagcatagccaaaaggcaaataaaatatcataccaatcttcaagtacggaagaaagacgactccactatggagaaaatggtgtgcagaaagatgtatcccaacgcagtatttatagccaaactgaaaagctagtagcaggcaagtctcaaatccaggcaccaaatcctaagcaagagccatggcatggtgaaaatgcaaaaggagagtctggccaatctacaaatagagaacaagacctactcagtcatgaacaaaaaggcagacaccaacatggatctcatgggggattggatattgtaattatagagcaggaagatgacagtgatcgtcatttggcacaacatcttaacaacgaccgaaacccattatttacataaacctaccattcggtaaccatgtgaaaggatggaccaatatcaaggtgtcagttgacctcagtgaattctgtgatgtttctgagatgcagactcccgtgtagtttcagattcttggtccatggatgacaccacctgcccatgcttccttgaattaggctttcctaacctgaagcgccttcaaacttccaataaagagatcattttctgcttcaaaaaaaaaaaaaaaaaaaaaaaaaaaa

SEQ ID NO: 894 - DNA, 837 bp, Homo sapiens
gctcctcctgcacacctccctcgctctcccacaccactggcaccaggccccggacacccgctctgctgcaggagaatggctactcatcacacgctgtggatgggactggccctgctgggggtgctgggcgacctgcaggcagcaccggaggcccaggtctccgtgcagcccaacttccagcaggacaagttcctggggcgctggttcagcgcgggcctcgcctccaactcgagctggctccgggagaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaataggactccccagggctgaagctgggatcccggccagccaggtgacccccacgctctggatgtctctgctctgttccttccccgagcccctgccccggctccccgccaaagcaaccctgcccactcaggcttcatcctgcacaataaactccggaagcaagtcagtaaaaaaaaaaaaaaaaaaaaaaaaaa

SEQ ID NO: 895 - DNA, 2390 bp, Homo sapiens
agagccttcgtttgccaagtcgcctccagaccgcagacatgaaacttgtcttcctcgtcctgctgttcctcggggccctcggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 896 - DNA, 1847 bp, Homo sapiens
gtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactc

SEQ ID NO: 897 - DNA, 626 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 898 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 899 - DNA, 1832 bp, Homo sapiens
gagcggccaggccagcctcggagccagcagggagctgggagctgggggaaacgacgccaggaaagctatcgcgccagagagggcgacgggggctcgggaagcctgacagggcttttgcgcacagctgccggctggctgctacccgcccgcgccagcccccgagaacgcgcgaccaggcacccagtccggtcaccgcagcggagagctcgccgctcgctgcagcgaggcccggagcggccccgcagggaccctccccagaccgcctgggccgcccggatgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttctgaacgtcccctgcccctttacggacaccccctcgcttggacggctgggcacacgcctcccactggggtccagggagcaggcggtgggcacccaccctgggacctaggggcgccgcaaaccacactggactccggccctcctaccctgcgcccagtccttccacctcgacgtttacaagcccccccttccacttttttttgtatgttttttttctgctggaaacagactcgattcatattgaatataatatatttgtgtatttaacagggaggggaagagggggcgatcgcggcggagctggccccgccgcctggtactcaagcccgcggggacattgggaaggggacccccgccccctgccctcccctctctgcaccgtactgtggaaaagaaacacgcacttagtctctaaagagtttattttaagacgtgtttgtgtttgtgtgtgtttgttctttttattgaatctatttaagtaaaaaaaaaattggttctttaaaaaaaaaaaaaaaaa

SEQ ID NO: 900 - DNA, 1061 bp, Homo sapiens
tgtgaaggaaatcgggggaggaggatggacacaacatcccatctttgtgtttcgatacagactaagcttttaggccaaccctcctgactggatgggggcggcgggcgtggcatgcatgaaaagtaaacatcagagacctgaagaagcttataaaatagcttgggagaggccagtcaccaagacaggcatctcaaatcggctgattctgcatctggaaactgccttcatcttgaaagaaaagctccaggtcccttctccagccacccagccccaagatggtgatgctgctgctgctgctttccgcactggctggcctcttcggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccaggccaactactcactaatggaaaacggaaagatcaaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtacctgcatcatccaactttttcacgtggattttgcttggatcttggcaagaaaccctaatctccctccagaaacagtggactctctaaaaaatatcctgacttctaataacattgatgtcaagaaaatgacggtcacagaccaggtgaactgccccaagctctcgtaaccaggttctacagggaggctgcacccactccatgttacttctgcttcgctttcccctaccccaccccccccccataaagacaaaccaatcaaccacgacaaaggaagttgacctgaacatgtaaccatgccctaccctgttaccttgctagctgcaaaataaacttgttgctgacctgctgtgctcgcagtagattccaaaaaaaaaaaaaaaaaa

SEQ ID NO: 901 - DNA, 20 bp, Homo sapiens
agtgacggtaagccagtcag

SEQ ID NO: 902 - DNA, 20 bp, Homo sapiens
tccactttaatttcgggtca

SEQ ID NO: 903 - DNA, 20 bp, Homo sapiens
gaaagggaaagggtcaaaaa

SEQ ID NO: 904 - DNA, 20 bp, Homo sapiens
cacatctgcaagtacgttcg

SEQ ID NO: 905 - DNA, 20 bp, Homo sapiens
aagagctatgagctgcctga

SEQ ID NO: 906 - DNA, 20 bp, Homo sapiens
tacggatgtcaacgtcacac

SEQ ID NO: 907 - DNA, 3542 bp, Homo sapiens
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaa

SEQ ID NO: 908 - DNA, 3665 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

(19) United States
(12) Patent Application Publication
(10) Pub. No.: US 2012/0115138 A1
(43) Pub. Date: May 10, 2012

(54) METHOD FOR IN VITRO DIAGNOSING A COMPLEX DISEASE

(75) Inventors: Hans-Peter Deigner, Lampertheim (DE); Matthias Kohl, Rottweil (DE); Matthias Keller, Essen (DE); Therese Koal, Innsbruck (AT); Klaus Wwinberger, Mieming (AT)

(73) Assignee: BIOCRATES LIFE SCIENCES AG, Innsbruck (AT)

(21) Appl. No.: 13/263,429

(22) PCT Filed: Mar. 31, 2010
(86) PCT No.: PCT/EP2010/054384

§ 371 Date: Dec. 23, 2011

(30) Foreign Application Priority Data
Apr. 7, 2009 (EP) 09157517.5

(51) Int. Cl. C12Q 1/68; G01N 33/53

(52) U.S. Cl. 435/6.11; 435/6.12; 435/7.92

(57) ABSTRACT

The present invention relates to a method and kit for in vitro diagnosing a complex disease such as cancer, in particular, acute myeloid leukemia (AML), colon cancer, kidney cancer, prostate cancer; transient ischemic attack (TIA), ischemia, in particular stroke, hypoxia, hypoxic-ischemic encephalopathy, perinatal brain damage, hypoxic-ischemic encephalopathy of neotatals asphyxia; demyelinating disease, in particular, white-matter disease, periventricular leukoencephalopathy, multiple sclerosis, Alzheimer and Parkinson's disease; in a biological sample. For the diagnosis, use is made of measuring at least two different species of biomolecules and classifying the results by means of suitable classifier algorithms and other statistical procedures. With the present invention, a significant improvement of the reliability of e.g. expression profiles alone, are achieved. In other words, in a defined collective, an up to 100% accurate positive diagnosis could be achieved, which renders the method of the present invention superior over the prior art.

The present invention relates to a method for in vitro diagnosing a complex disease or subtypes thereof in accordance with claim 1 and to a Kit for carrying out the method in accordance with claim 18.

In classical patient screening and diagnosis, the medical practitioner uses a number of diagnostic tools for diagnosing a patient suffering from a certain disease. Among these tools, measurement of a series of single routine parameters, e.g. in a blood sample, is a common, diagnostic laboratory approach. These single parameters comprise for example enzyme activities and enzyme concentration and/or detection of metabolic indicators such as glucose and the like. As far as such diseases are concerned which easily and unambiguously can be correlated with one single parameter or a few number of parameters achieved by clinical chemistry, these parameters have proved to be indispensable tools in modern laboratory medicine and diagnosis. Under the provision that excellently validated cut-off values can be provided, such as in the case of diabetes, clinical chemical parameters such as blood glucose can be reliably used in diagnosis.

In particular, when investigating pathophysiological states underlying essentially a well known pathophysiological mechanism, from which the guiding parameter is resulting, such as a high glucose concentration in blood typically reflects an inherited defect of an insulin gene, such single parameters have proved to be reliable biomarkers for “its” diseases.

However, in pathophysiological conditions, such as cancer or demyelinating diseases such as multiple sclerosis which share a lack of an unambiguously assignable single parameter or marker, differential diagnosis from blood or tissue samples is currently difficult to impossible.

In cancer prevention, screening, diagnosis, treatment and aftertreatment, it is meanwhile clinical routine to use a series of so called “tumor markers” each being somewhat specific for a certain kind of cancer to diagnose and to monitor therapy of malign processes. Such currently used tumor markers are for example Alpha-1-fetoprotein, cancer antigen 125 (CA 125), cancer antigen 15-3, CA 50, CA 72-4, carbohydrate antigen 19-9, calcitonin, carcino embryonic antigen (CEA), cytokeratine fragment 21-1, mucin-like carcinoma-associated antigen, neuron specific enolase, nuclear matrix protein 22, alkaline phosphatase, prostate specific antigen (PSA), squamous cell carcinoma antigen, telomerase, thymidine kinase, Thyreoglobulin, and tissue polypeptid antigen.

Although, in the prior art already a number of the above tumor markers are meanwhile routinely used it very often is difficult from a single measurement to achieve a reliable diagnosis. Just by way of example, the cut-off values of the CEA is 4.6 ng/ml for non-smokers, whereas 25% of smokers show normal values in the range of 3.5 to 10 ng/ml and 1% of smokers show normal values of more than 10 ng/ml. Thus, only values above 20 ng/ml have to be interpreted as being “highly suspicious for a malign process”, which leaves a significant grey zone in which the physician cannot rely upon the CEA-values measured in a patient's sample.

EP 540 573 B1 discloses similar cut-off values' problems with respect to the prostate specific antigen (PSA) in which typically total PSA is measured for diagnosing or excluding prostate cancer in a patient, and if the values are in the grey zone, it is the current approach to measure in addition to total PSA also free PSA with a monoclonal antibody assay being specific for free PSA and calculate a ratio of both parameters in order to get a more accurate approach for diagnosing prostate cancer and to differentiate from benign prostate hyperplasia.

The above examples of CEA and PSA detection impressively demonstrate what is common with all single tumor markers, namely on one hand, the relatively poor specificity, and on the other hand, uncertain and unreliable cut-off values so that the achieved values are difficult to interpret.

Thus, as a general consequence, it is recommended to consider the use of tumor markers in screening as critical. It is not rarely that increased levels of tumor markers without further clinical correlation lead to unnerving of the patients and do not have any diagnostic value at all.

Furthermore, in aftertreatment of malign diseases, it has to be noticed that every tumor marker needs a “critical mass” of cancer cells first, until it responds positively in clinical test. In addition, not every recurrent tumor must involve an increase of tumor marker levels.

In summary, single tumor markers proved to be useful in clinical practice only mostly in context with other diagnostic tools such as endoscopy and biopsy, followed by histological examination, but are not reliable in routine cancer screening.

Vis-á-vie the prior art of single tumor markers, it was a great progress to use gene expression levels of a plurality of genes with the microarray technology.

WO 2004111197A2, e.g. discloses minimally invasive sample procurement method for obtaining airway epithelial cell RNA that can be analyzed by expression profiling, e.g., by array-based gene expression profiling. These methods can be used to identify patterns of gene expression that are diagnostic of lung disorders, such as cancer, to identify subjects at risk for developing lung disorders and to custom design an array, e.g., a microarray, for the diagnosis or prediction of lung disorders or susceptibility to lung disorders. Arrays and informative genes are also disclosed for this purpose.

Such multiple gene approaches are much more reliable then the above mentioned single parameters, however, are subject to complex mathematical and bioinformatics procedures. Nevertheless, these gene expression signatures are promising tools in cancer diagnosis, but sometimes also have uncertainty limits what leads due to their underlying statistics and being restricted to one kind nucleic acids also to sometimes unreliable results and validation problems.

Staring from the above mentioned prior art, it is the problem of the present invention to provide a use of biomarkers in diagnostics tools with the highest possible sensitivity and specificity for early diagnosis to identify diseased subjects, for use in patient pre-selection and stratification and for therapy control is a main goal in diagnostic development and still an urgent need in various complex diseases, in particular cancer.

The above problem is solved by a method in accordance with claim 1 and a kit in accordance with claim 18.

In particular, the present invention provides a method for in vitro diagnosing a complex disease or subtypes thereof, selected from the group consisting of:

cancer, in particular, acute myeloid leukemia (AML), colon cancer, kidney cancer, prostate cancer; ischemia, in particular stroke, hypoxia, hypoxic-ischemic encephalopathy, perinatal brain damage, hypoxic-ischemic encephalopathy of neotatals asphyxia; demyelinating disease, in particular, white-matter disease, periventricular leukoencephalopathy, multiple sclerosis;
in at least one biological sample of at least one tissue of a mammalian subject comprising the steps of:
a) selecting at least two different species of biomolecules, wherein said species of biomolecules are selected from the group consisting of RNA and/or its DNA counterparts, microRNA and/or its DNA counterparts, peptides, proteins, and metabolites;
b) measuring at least one parameter selected from the group consisting of presence (positive or negative), qualitative and/or quantitative molecular pattern and/or molecular signature, level, amount, concentration and expression level of a plurality of biomolecules of each species in said sample using at least two sets of different species of biomolecules and storing the obtained set of values as raw data in a database;
c) mathematically preprocessing said raw data in order to reduce technical errors being inherent to the measuring procedures used in step b);
d) selecting at least one suitable classifying algorithm from the group consisting of logistic regression, (diagonal) linear or quadratic discriminant analysis (LDA, QDA, DLDA, DQDA), perceptron, shrunken centroids regularized discriminant analysis (RDA), random forests (RF), neural networks (NN), Bayesian networks, hidden Markov models, support vector machines (SVM), generalized partial least squares (GPLS), partitioning around medoids (PAM), self organizing maps (SOM), recursive partitioning and regression trees, K-nearest neighbor classifiers (K-NN), fuzzy classifiers, bagging, boosting, and naïve Bayes; and applying said selected classifier algorithm to said preprocessed data of step c);
e) said classifier algorithms of step d) being trained on at least one training data set containing preprocessed data from subjects being divided into classes according to their pathophysiological, physiological, prognostic, or responder conditions, in order to select a classifier function to map said preprocessed data to said conditions;
f) applying said trained classifier algorithms of step e) to a preprocessed data set of a subject with unknown pathophysiological, physiological, prognostic, or responder condition, and using the trained classifier algorithms to predict the class label of said data set in order to diagnose the condition of the subject.

Dependant claims 2 to 18 are preferred embodiments of the present invention.

The present invention provides a solution to the problem described above, and generally relates to the use of “omics” data comprising, but not limited to mRNA expression data, microRNA expression data, proteomics data, and metabolomics data, statistical learning respectively machine learning for identification of molecular signatures and biomarkers. It comprises the determination of the concentrations of the aforementioned biomolecules via known methods such as polymerase chain reaction (PCR), microarrays and other methods such as sequencing to determine RNA concentrations, protein identification and quantification by mass spectrometry (MS), in particular MS-technologies such as MALDI, ESI, atmospheric pressure pressure chemical ionization (APCI), and other methods, determination of metabolite concentrations by use of MS-technologies or alternative methods, subsequent feature selection and the combination of these features to classifiers including molecular data of at least two molecular levels (that is at least two different types of endogenous biomolecules, e.g. RNA concentrations plus metabolomics data respectively concentrations of metabolites or RNA concentrations plus concentrations of proteins or peptides etc.) and optimal composed marker sets are extracted by statistical methods and data classification methods.

The concentrations of the individual markers of the distinct molecular levels (RNA molecules, peptides/proteins, metabolites etc.) thus are measured and data processed to classifiers indicating diseased states etc. with superior sensitivities and specificities compared to procedures and biomarker confined to one type of biomolecules.

A method for the selection and combination of biomarkers and molecular signatures of biomolecules in particular utilizing one or several individual molecules of the biomolecule types mRNA, microRNA, proteins, or peptides, small endogenous compounds (metabolites) in combination (combining at least two of the aforementioned types of biomolecules), with the biomolecules obtained from body liquids or tissue, identified by use of statistical methods and classifiers derived from the data of these groups of molecules for use in diagnosis and early diagnosis, for patient stratification, therapy selection, therapy monitoring and theragnostics in complex diseases is described.

### BACKGROUND OF THE INVENTION

### Prior Art

Systems biology approaches utilizing varying omics approaches such as genomics, proteomics and metabolomics are increasingly applied to research and diagnostics of complex diseases. These technologies may provide data and biological indicators, so-called (prognostic, predictive and pharmacodynamic) biomarkers with the potential to revolutionize clinical practice in diagnosis.

For early cancer detection single biomarkers are commonly used. However, the widely used cancer antigen 125 (CA125) for instance can only detect 50%-60% of patients with stage I ovarian cancer. Analogously, the single use of the prostate specific antigen (PSA) value for early stage prostate cancer identification is not specific enough to reduce the number of false positives [Petricoin E F 3rd, Ornstein D K, Paweletz C P, Ardekani A, Hackett P S, Hitt B A, Velassco A, Trucco C, Wiegand L, Wood K, Simone C B, Levine P J, Linehan W M, Emmert-Buck M R, Steinberg S M, Kohn E C, Liotta L A, Serum proteomic patterns for detection of prostate cancer, J Natl Cancer Inst. 2002; 94(20):1576-8] and it is evident that it is highly unlikely that a complex disease can be characterized or diagnosed and the effect of therapies assessed by use of single biomarkers.

Recent advances in diagnostic tools e.g. in cancer diagnostics typically comprise multi-component tests utilizing several biomarkers of the same class of biomolecules such as several proteins, RNA or microRNA species and the analysis of high dimensional data gives a deeper insight into the abnormal signaling and networking which has a high potential to identify previously not discovered marker candidates. However, methods according to the present state of the art utilize single biomolecules or sets of a single type of biomolecules for biomarkers sets such as several RNA, microRNA or protein molecules. See Garzon R, Volinia S, Liu C G, Fernandez-Cymering C, Palumbo T, Pichiorri F, Fabbri M, Coombes K, Alder H, Nakamura T, Flomenberg N, Marcucci G, Calin G A, Kornblau S M, Kantarjian H, Bloomfield C D, Andreeff M, Croce C M, MicroRNA signatures associated with cytogenetics and prognosis in acute myeloid leukemia, Blood. 2008; 111(6):3183-9 and Ramaswamy S, Tamayo P, Rifkin R, Mukherjee S, Yeang C H, Angelo M, Ladd C, Reich M, Latulippe E, Mesirov J P, Poggio T, Gerald W, Loda M, Lander E S, Golub T R., Multiclass cancer diagnosis using tumor gene expression signatures. Proc Natl Acad Sci USA. 2001; 98(26):15149-54. For miRNA in Cancer see WO2008055158.

In addition, Oncotype DX is an example of a recent multicomponent RNA-based test, like a multigene activity assay, to predict recurrence of tamoxifen-treated, node-negative breast cancer is disclosed in Paik S, Shak S, Tang G, Kim C, Baker J, Cronin M, Baehner F L, Walker M G, Watson D, Park T, Hiller W, Fisher E R, Wickerham D L, Bryant J, Wolmark N, Engl J. Med. 2004; 351(27):2817-26.

Habel L A, Shak S, Jacobs M K, Capra A, Alexander C, Pho M, Baker J, Walker M, Watson D, Hackett J, Blick N T, Greenberg D, Fehrenbacher L, Langholz B, Quesenberry C P describe a population-based study of tumor gene expression and risk of breast cancer death among lymph node-negative patients in Breast Cancer Res. 2006; 8(3):R25.

Other recent examples include breast-cancer gene-expression signatures—marketed for clinical use as), MammaPrint (Agendia).

Furthermore, Glas A M, Floore A, Delahaye L J, Witteveen A T, Pover R C, Bakx N, Lahti-Domenici J S, Bruinsma T J, Warmoes M O, Bernards R, Wessels L F, Van't Veer L J. Disclose a method for converting a breast cancer microarray signature into a high-throughput diagnostic test in BMC Genomics. 2006; 7:278.

Another known approach is disclosed as the so called H/I test (AviaraDx), developed by Nicholas C Turner and Alison L Jones BMJ. 2008 Jul. 19; 337(7662): 164-169, which estimates the probability of the original breast cancer recurring after it has been resected.

Although these products and prototypes demonstrate significant progress for specific areas of diagnostics, there is still an urgent need for reliable and early diagnostics with high sensitivities and specificities in a number of complex diseases such as, but not limited to, cancer, in particular, acute myeloid leukemia (AML), colon cancer, kidney cancer, prostate cancer; ischemia, in particular stroke, hypoxia, hypoxic-ischemic encephalopathy, perinatal brain damage, hypoxic-ischemic encephalopathy of neotatals asphyxia; demyelinating disease, in particular, white-matter disease, periventricular leukoencephalopathy, multiple sclerosis, Alzheimer and Parkinson disease. These diagnostic tools and biomarkers are also being used for the selection of responders among patients, for an assessment of disease recurrence, the selection of therapeutic options, efficacy, drug resistance and toxicity.

The invention provides the principle and the method for the generation of novel diagnostic tools to diagnose complex diseases with superior sensitivities and specificities to address these problems.

Data integration of various “omics” data, e.g. to identify possible alterations of protein concentrations from altered RNA transcripts is an issue familiar to systems biology and to persons skilled in the arts for years.

Despite of that, the statistical combination of biomarker sets from different types of biomolecules, independent of data integration and biochemical interpretation to combined diagnostic signatures (combining several types of biomolecules) on a statistical basis applying various classification methods as described here is not obvious, unknown to persons skilled in the art, and has not been described in the literature. It clearly is distinct to approaches utilizing an integrative multi-dimensional analysis and combining e.g. genomes, epigenomes and transcriptomes (see SIGMA2: A system for the integrative genomic multi-dimensional analysis of cancer genomes, epigenomes, and transcriptomes, Raj Chari et al. BMC Bioinformatics 2008, 9:422) which attempt to analyse biological relationships between different omics data by various means.

Essentially, the method according to the present invention combines statistically significant biomolecule parameters of at least two different types of biomolecules on a statistical basis, entirely irrespective of known or unknown biological relationship of any kind, links or apparent biological plausibility to afford a combined biomarker composed of several types of biomolecules. The patient cases underlying the invention demonstrate that a diagnostic method and disease state specific classifier composed of at least two of the aforementioned biomolecule types and those combined biomolecules of at least two types describing the respective state of cells, a tissue, an organ or an organisms best among a collective of measured molecules, is superior to a composition of molecules or markers and their delineated molecular signatures. It is further superior to classifiers of biomolecules of just one type of biomolecules and as demonstrated here yields higher sensitivities and specificities in diagnostic applications. In that the present invention goes far beyond the current state of the art and provides a method for generating diagnostic molecular signatures affording higher sensitivities and specificities and decreased false discovery rates compared to methods available so far. The method can be applied for diagnosing various complex and completely unrelated complex diseases such as cancer and ischemia and is of general diagnostic use.

### DETAILED DESCRIPTION OF THE INVENTION

### Definitions

As used herein, the term “gene expression” refers to the process of converting genetic information encoded in a gene into ribonucleic acid, RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production

Polynucleotide: A nucleic acid polymer, having more, than 2 bases.

“Peptides” are short heteropolymers formed from the linking, in a defined order, of α-amino acids. The link between one amino acid residue and the next is known as an amide bond or a peptide bond.

Proteins are polypeptide molecules (or consist of multiple polypeptide subunits). The distinction is that peptides are short and polypeptides/proteins are long. There are several different conventions to determine these, all of which have caveats and nuances.

A “Complex disease” within the scope of the present invention is one belonging to the following group, but is not limited to this group: cancer, in particular, acute myeloid leukemia (AML), colon cancer, kidney cancer, prostate cancer; transient ischemic attack (TIA), ischemia, in particular stroke, hypoxia, hypoxic-ischemic encephalopathy, perinatal brain damage, hypoxic-ischemic encephalopathy of neotatals asphyxia; demyelinating disease, in particular, white-matter disease, periventricular leukoencephalopathy, multiple sclerosis, Alzheimer and Parkinson's disease.

Metabolite: as used here, the term “metabolite” denotes endogenous organic compounds of a cell, an organism, a tissue or being present in body liquids and in extracts obtained from the aforementioned sources with a molecular weight typically below 1500 Dalton. Typical examples of metabolites are carbohydrates, lipids, phospholipids, sphingolipids and sphingophospholipids, amino acids, cholesterol, steroid hormones and oxidized sterols and other compounds such as collected in the Human Metabolite database (http://www.hmdb.ca/) and other databases and literature. This includes any substance produced by metabolism or by a metabolic process and any substance involved in metabolism.

“Metabolomics” as understood within the scope of the present invention designates the comprehensive quantitative measurement of several (2-thousands) metabolites by, but not limited to, methods such as mass spectroscopy, coupling of liquid chromatography, gas chromatography and other separation methods chromatography with mass spectroscopy.

“Oligonucleotide arrays “or” oligonucleotide chips” or “gene chips”: relates to a “microarray”, also referred to as a “chip”, “biochip”, or “biological chip”, is an array of regions having a suitable density of discrete regions, e.g., of at least 100/cm², and preferably at least about 1000/cm². The regions in a microarray have dimensions, e.g. diameters, preferably in the range of between about 10-250 μm, and are separated from other regions in the array by the same distance. Commonly used formats include products from Agilent, Affymetrix, Illumina as well as spotted fabricated arrays where oligonucleotides and cDNAs are deposited on solid surfaces by means of a dispenser or manually.

It is clear to a person skilled in the art that nucleic acids, proteins and peptides as well as metabolites can be quantified by a variety of methods including the above mentioned array systems as well as but not limited to: quantitative sequencing, quantitative polymerase chain reaction and quantitative reverse transcription polymerase chain reaction (qPCR and RT-PCR), immunoassays, protein arrays utilizing antibodies, mass spectrometry.

“microRNAs” (miRNAs) are small RNAs of 19 to 25 nucleotides that are negative regulators of gene expression. To determine whether miRNAs are associated with cytogenetic abnormalities and clinical features in acute myeloid leukemia (AML), the miRNA expression of CD34(+) cells and 122 untreated adult AML cases is evaluated using a microarray platform.

Under different species or types or classes of biomolecules in this context is understood: RNA, microRNA, proteins and peptides of various lengths as well as metabolites.

A biomarker in this context is a characteristic, comprising data of at least two biomolecules of at least two different types (RNA, microRNA, proteins and peptides, metabolites) that is measured and evaluated as an indicator of biologic processes, pathogenic processes, or responses to an therapeutic intervention. A combined biomarker as used here may be selected from at least two of the following types of biomolecules: sense and antisense nucleic acids, messenger RNA, small RNA i.e. siRNA and microRNA, polypeptides, proteins including antibodies, small endogenous molecules and metabolites.

Data classification is the categorization of data for its most effective and efficient use. Classifiers are typically deterministic functions that map a multi-dimensional vector of biological measurements to a binary (or n-ary) outcome variable that encodes the absence or existence of a clinically-relevant class, phenotype, distinct physiological state or distinct state of disease. To achieve this various classification methods such as, but not limited to, logistic regression, (diagonal) linear or quadratic discriminant analysis (LDA, QDA, DLDA, DQDA), perceptron, shrunken centroids regularized discriminant analysis (RDA), random forests (RF), neural networks (NN), Bayesian networks, hidden Markov models, support vector machines (SVM), generalized partial least squares (GPLS), partitioning around medoids (PAM), self organizing maps (SOM), recursive partitioning and regression trees, K-nearest neighbor classifiers (K-NN), fuzzy classifiers, bagging, boosting, and naïve Bayes and many more can be used.

The term “binding”, “to bind”, “binds”, “bound” or any derivation thereof refers to any stable, rather than transient, chemical bond between two or more molecules, including, but not limited to covalent bonding, ionic bonding, and hydrogen bonding. Thus, this term also encompasses hybridization between two nucleic acid molecules among other types of chemical bonding between two or more molecules.

### DESCRIPTION

In the method of the present invention, biomarker data and classifier obtained by combination of at least two different types of biomolecules out of two different species of biomolecules, wherein said species of biomolecules are selected from the group consisting of: RNA and/or its DNA counterparts, microRNA and/or its DNA counterparts, peptides, proteins, and metabolites, identified according to the invention afford a description of a physiological state and can be used as a superior tool for diagnosing complex diseases.

The discrimination of pathological samples or tissues from healthy specimens requires a combination of data of at least two distinct types of biomolecules, a determination of their concentrations and a statistical processing and classifier generation according to the method depicted in Table 1 below.

As mentioned above a biological link between molecules combined in a biomarker by means of classification is entirely irrelevant to the outcome and selection of the issues and can not be necessarily explained by biological models.

The method according to the present invention comprises essentially the following steps:

First, a biological sample obtained from a subject or an organism is obtained.
Second, the amounts of biomolecules of the following types (RNA, microRNA, peptide or protein, metabolite) are measured from the biological sample and stored as raw data in a database.
Third the raw data from the database are preprocessed.
Fourth, the amount of RNA and/or its DNA counterparts, microRNA and/or its DNA counterparts, peptide or protein, metabolite detected in the sample is compared to either a standard amount of the respective biomolecule measured in a normal cell or tissue or a reference amount of the respective biomolecule stored in a database. If the amount of the biomolecules of interest in the sample is different to the amount of the biomolecules determined in the standard or control sample, the differential concentration data are processed and used for step 5 classifier generation as described below.

The classifier is validated in step 6 and used in step 7: according to the invention, the classifier utilizes data from at least two groups of biomolecules of the aforementioned types and afford a value or a score. This score is assigned to an altered physiological state of plasma, tissue or an organ with a computed probability and can indicate a diseased state, a state due to intervention (e.g. therapeutic intervention by treatment, surgery or pharmacotherapy) or an intoxication with some probability. This score can be used as a diagnostic tool to indicate that the subject or the organism is diagnosed as diseased, to indicate intoxication as having cancer.

The score and time-dependent changes of the score can be used to assess the success of a treatment or the success of a drug administered to the subject or the organism or assess the individual response of a subject or an organism to the treatment or to make a prognosis of the future course of the physiological state or the disease and the outcome. The prognoses are relative to a subject without the disease or the intoxication having normal levels or average values of the score or classifier composed of at least two biomolecules

TABLE 1
Table 1: Schematic diagram of proposed method. More details
are given in text.

Step 1:
Biological sample obtained
Step 2:
Measurement of raw data (concentrations of
biomolecules) and deposit in data base
Step 3:
Preprocessing of raw data from data base
Step 4:
Comparison to reference values and feature selection
Step 5:
Train classifier based on data of a composed biomarker
composed of at least two types of biomolecules
Step 6:
Validate classifier
Step 7:
Use of the classifier to assess physiological state, as
diagnostic tool to indicate a diseased state or as a
prognostic tool

In case of mRNA and microRNA data the preprocessing of the data typically consists of background correction and normalization. The skilled person is aware of a number of suitable known background correction and normalization strategies; a comparative survey in case of Affymetrix data is given in L. M. Cope et al., A Benchmark for Affymetrix GeneChip Expression Measures, Bioinformatics 2004, 20(3), 323-331 or R. A. Irizarry et al., Comparison of Affymetrix GeneChip Expression Measures, Bioinformatics 2006, 22(7), 789-794, respectively.

Depending on the data at hand, it may also consist of some variance stabilizing transformation or transformation to normality as for instance taking the logarithm or using Box-Cox power transformations [Box, G. E. P. and Cox, D. R. An analysis of transformations (with discussion). Journal of the Royal Statistical Society B 1964, 26, 211-252].

Often also scaling e.g. by standard deviation or median absolute deviation (MAD) might be used to transform the raw data. However, this step is not necessary for all kind of data, respectively all kind of further statistical analyses and hence may also be omitted.

The feature (variable, measurement) selection step might also be optional. However, it is recommended if the number of features is larger than the number of samples. Feature selection methods try to find the subset of features with the highest discriminatory power.

Due to the high dimensionality of mRNA and microRNA data, most classification algorithms cannot be directly applied. One reason is the so-called curse of dimensionality: With increasing dimensionality the distances among the instances assimilate. Noisy and irrelevant features further contribute to this effect, making it difficult for the classification algorithm to establish decision boundaries. Further reasons why classification algorithms are not applicable on the full dimensional space are performance limitations. Ultimately, feature transformation techniques are applied before classification, e.g. in [J. S. Yu et al., Ovarian cancer identification based on dimensionality reduction for high-throughput mass spectrometry data, Bioinformatics, 21(10):2200-2209, 2005]. Furthermore, also for the task of identifying unknown marker candidates, the use of traditional methods is limited due to the high dimensionality of the data.

To identify diseased subjects with the highest possible sensitivity and specificity is the main goal in diagnostic development. For this purpose, a large number of classification algorithms can be chosen e.g. logistic regression, (diagonal) linear or quadratic discriminant analysis (LDA, QDA, DLDA, DQDA), shrunken centroids regularized discriminant analysis (RDA), random forests (RF), neural networks (NN), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more can be applied to develop new marker candidates. These algorithms are trained on at least one training data set which contains instances labeled according to classes, e.g. healthy and diseased, and then tested on at least one test data set which includes novel instances not used for the training. In the training-test step one or more rounds of cross-validation, bootstrap or some split-sample approach can be used to estimate how accurately a predictive model will perform in practice. Finally, the classifier will be used to predict the class label of novel unlabeled instances [T. M. Mitchell. Machine Learning. McGraw-Hill, 1997].

Classifiers are typically deterministic functions that map a multi-dimensional vector of biological measurements to a binary (or n-ary) outcome variable that encodes the absence or existence of a clinically-relevant class, phenotype or distinct state of disease. The process of building or learning a classifier involves two steps: (1) selection of a family functions that can approximate the systems response, and using a finite sample of observations (training data) to select a function from the family of functions that best approximates the system's response by minimizing the discrepancy or expected loss between the system's response and the function predictions at any given point.

Depending on the chosen feature selection strategy, the combination of the different data (clinical data, mRNA, microRNA, metabolites, proteins) can take place before or after feature selection. The combined data is then used as input to train and validate the classifier. However, it is also possible to train several different classifiers for the different data separately and then combine the classifiers to the predictive signature. As the data types may be very different from qualitative/categorical to quantitative/numerical, not all classifiers may work for such multilevel data; e.g., some classifiers accept only quantitative data. Hence, depending on the data types one has to choose a class of functions for classification which has an appropriate domain.

Numerous feature selection strategies for classification have been proposed, for a comprehensive survey see e.g. [M. A. Hall and G. Holmes, Benchmarking Attribute Selection Techniques for Discrete Class Data Mining.

IEEE Transactions on Knowledge and Data Engineering, 15(6): 1437-1447, 2003.]. Following a common characterization, it is distinguished between filter and wrapper approaches.

Filter approaches use an evaluation criterion to judge the discriminating power of the features. Among the filter approaches, it can further be distinguished between rankers and feature subset evaluation methods. Rankers evaluate each feature independently regarding its usefulness for classification. As a result, a ranked list is returned to the user. Rankers are very efficient, but interactions and correlations between the features are neglected. Feature subset evaluation methods judge the usefulness of subsets of the features. The information of interactions between the features is in principle preserved, but the search space expands to the size of O (2<d>). For high-dimensional data, only very simple and efficient search strategies, e.g. forward selection algorithms, can be applied because of the performance limitations.

The wrapper attribute selection method uses a classifier to evaluate attribute subsets. Cross-validation is used to estimate the accuracy of the classifier on novel unclassified objects. For each examined attribute subset, the classification accuracy is determined. Adapted to the special characteristics of the classifier, in most cases wrapper approaches identify attribute subsets with higher classification accuracies than filter approaches, cf. Pochet, N., De Smet, F., Suykens, J. A., and De Moor, B. L., Systematic benchmarking of microarray data classification: assessing the role of non-linearity and dimensionality reduction. Bioinformatics, 20(17):3185-95 (2004). As the attribute subset evaluation methods, wrapper approaches can be used with an arbitrary search strategy. Among all feature selection methods, wrappers are the most computational expensive ones, due to the use of a learning algorithm for each examined feature subset.

A preferred embodiment of the present invention is a method, wherein said complex disease is AML, said mammalian subject is a human being, said biological sample blood and/or blood cells and/or bone marrow;

wherein said different species of biomolecules are microRNA and proteins, in particular surface proteins from non-mature hematopoietic stem cells, preferably CD34;

wherein microRNA expression levels and CD34 presence are used as said parameters of step b);

wherein raw data of microRNA expression are preprocessed using a variance-stabilizing normalization and summarizing the normalized multiple probe signals (technical replicates) to a single expression value, using the median;

wherein a ranker, in particular a Mann-Whitney significance test combined with largest median of pairwise differences as filter for microRNA expression data is used for said feature selection;

wherein logistic regression is selected as suitable classifying algorithm, the training of the classifying algorithm including preprocessed and filtered microRNA expression data and CD34 information (positive or negative), is carried out with an n-fold cross-validation, in particular 5 to 10-fold, preferably 5-fold cross-validation;

applying said trained logistic regression classifier to said preprocessed microRNA expression data set and CD34 information to a subject under suspicion of having AML, and using the trained classifiers to diagnose a specific AML-type.

Another preferred embodiment of the present invention is a method, wherein said complex disease is colon cancer, said mammalian subject is a human being, said biological sample is colon tissue;

wherein said different species of biomolecules are mRNA and/or its DNA counterparts and microRNA and/or its DNA counterparts;

wherein mRNA expression levels and microRNA expression levels are used as said parameters of step b);

wherein raw data of microRNA expression are preprocessed using a variance-stabilizing normalization;

wherein raw data of mRNA expression are preprocessed using a variance-stabilizing normalization and summarizing the perfect match (PM) and miss match (MM) probes to an expression measure using a robust multi-array average (RMA);

wherein a ranker, in particular a Mann-Whitney significance test combined with largest median of pairwise differences as filter for microRNA expression data is used for said feature selection;

wherein random forests are selected as suitable classifying algorithm, the training of the classifying algorithm including preprocessed and filtered mRNA and microRNA expression data is carried out with a leave-one-out (LOO) cross-validation;

applying said trained random forests classifier to said preprocessed mRNA and microRNA expression data sets to a subject under suspicion of having colon cancer, and using the trained classifiers to diagnose colon cancer and/or a subtype thereof.

A further preferred embodiment of the present invention is a method, wherein said complex disease is kidney cancer, said mammalian subject is a human being, said biological sample is kidney tissue;

wherein said different species of biomolecules are mRNA and/or its DNA counterparts and microRNA and/or its DNA counterparts;

wherein mRNA expression levels and microRNA expression levels are used as said parameters of step b);

wherein raw data of microRNA expression are preprocessed using a variance-stabilizing normalization;

wherein raw data of mRNA expression are preprocessed using a variance-stabilizing normalization and summarizing the perfect match (PM) and miss match (MM) probes to an expression measure using a robust multi-array average (RMA);

wherein a ranker, in particular a Welch t-test (significance test) combined with largest mean of pairwise differences as filter for mRNA and microRNA expression data is used for said feature selection;

wherein single-hidden-layer neural networks are selected as suitable classifying algorithm, the training of the classifying algorithm including preprocessed and filtered mRNA and microRNA expression data, is carried out with a leave-one-out (LOO) cross-validation; applying said trained random forests classifier to said preprocessed mRNA and microRNA expression data sets to a subject under suspicion of having kidney cancer, and using the trained classifiers to diagnose kidney cancer and/or a subtype thereof.

Another preferred embodiment of the present invention is a method, wherein said complex disease is prostate cancer, said mammalian subject is a human being, said biological sample is urine and/or prostate tissue;

wherein said different species of biomolecules are mRNA and/or its DNA counterparts and microRNA and/or its DNA counterparts;

wherein mRNA expression levels and microRNA expression levels are used as said parameters of step b);

wherein raw data of microRNA expression are preprocessed using a variance-stabilizing normalization;

wherein raw data of mRNA expression are preprocessed using a variance-stabilizing normalization and summarizing the perfect match (PM) and miss match (MM) probes to an expression measure using a robust multi-array average (RMA);

wherein a ranker, in particular a Mann-Whitney significance test combined with largest median of pairwise differences as filter for mRNA and microRNA expression data is used for said feature selection;

wherein linear discriminant analysis is selected as suitable classifying algorithm, the training of the classifying algorithm including preprocessed and filtered mRNA and microRNA expression data, is carried out with a leave-one-out (LOO) cross-validation;

applying said trained random forests classifier to said preprocessed mRNA and microRNA expression data sets to a subject under suspicion of having prostate cancer, and using the trained classifiers to diagnose prostate cancer and/or a subtype thereof.

Again another preferred embodiment of the present invention is a method, wherein said complex disease is transient ischemic attack (TIA) and/or ischemia and/or hypoxia, said mammalian subject is a human being, said biological sample blood and/or blood cells and/or cerebrospinal fluid and/or brain tissue;

wherein said different species of biomolecules are mRNA and/or its DNA counterparts and brain metabolites, in particular free prostaglandins, lipooxygenase derived fatty acid metabolites, glutamine, glutamic acid, leucin, alanine, serine, decosahexaenoic acid (DHA), 12(S)-hydroxyeicosatetraenoic acid (12S-HETE);

wherein mRNA expression levels and quantitative and/or qualitative molecular metabolite patterns (metabolomics data) are used as said parameters of step b);

wherein raw data of mRNA expression are preprocessed using actin-β as reference genes and metabolomics data of said brain metabolites are preprocessed by a variance stabilizing transformation via the binary logarithm (i.e. to base 2);

wherein a ranker, in particular a Welch t-test (significance test) combined with largest mean of pairwise differences as filter for metabolomics data is used for said feature selection;

wherein support vector machines are selected as suitable classifying algorithm, the training of the classifying algorithm including preprocessed and filtered mRNA and microRNA expression data, is carried out with a leave-one-out (LOO) cross-validation;

applying said trained support vector machines classifier to said preprocessed mRNA expression data and said metabolomics data sets to a subject under suspicion of having ischemia and/or hypoxia, and using the trained classifiers to diagnose ischemia and/or hypoxia and/or the grades thereof.

### EXAMPLES

### Example 1

### Method Utilizing MicroRNA and Protein Data

As a first example, we use the microRNA and clinical data of Garzon R, Garofalo M, Martelli M P, Briesewitz R, Wang L, Fernandez-Cymering C, Volinia S, Liu C G, Schnittger S, Haferlach T, Liso A, Diverio D, Mancini M, Meloni G, Foa R, Martelli M F, Mecucci C, Croce C M, Falini B. Distinctive microRNA signature of acute myeloid leukemia bearing cytoplasmic mutated nucleophosmin. PNAS 2008, 105(10):3945-50.

These data are available in the ArrayExpress online database http://www.ebi.ac.uk/arrayexpress under accession number E-TABM-429. Overall the microRNA data of 85 adult de novo AML patients characterized for subcellular localization/mutation status of NPM1 and FLT3 mutations are available. The hybridizations' were done using the OSU-CCC human & mouse microRNA 11K v2 Microarray Shared Resource, Comprehensive Cancer Center, The Ohio State University (OSU-CCC).

Acute myeloid leukemia (AML) carrying NPM1 mutations and cytoplasmic nucleophosmin (NPMc+ AML) accounts for about one-third of adult AML and shows distinct features including a unique gene expression profile. The authors used microRNA expression values to distinguish NPMc+ mutated (n=55) from the cytoplasmic-negative (NPMc−, i.e., NPM1 unmutated) cases (n=30).

Analysis:

For developing and validating a classifier based on these data we used logistic regression in combination with 5-fold cross-validation where each analysis step—including low level analysis—was repeated in each cross-validation step. Moreover, we repeated 5-fold cross-validation 20 times. This is one possibility. Of course, we could also have used a split-sample, a bootstrap or a different k-fold (k not equal to 5) cross-validation approach. Moreover, we could have used a different class of functions for classification e.g. (diagonal) linear or quadratic discriminant analysis (LDA, QDA, DLDA, DQDA), shrunken centroids regularized discriminant analysis (RDA), random forests (RF), neural networks (NN), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more. The low level analysis consisted of the variance stabilizing transformation of Huber et al. (2002) [Huber W, von Heydebreck A, Sueltmann H, Poustka A, Vingron M. Variance Stabilization Applied to Microarray Data Calibration and to the Quantification of Differential Expression. Bioinformatics 2002, 18: 96-104] (often called normalization) and the averaging of the normalized replicates using the median. Again there is a large number of alternative methods which could be used. Several examples are given in L. M. Cope et al., Bioinformatics 2004, 20(3), 323-331 or R. A. Irizarry et al., Bioinformatics 2006, 22(7), 789-794. In each cross validation step we selected those five normalized and averaged microRNA probes for classification which had the largest median of pairwise differences (in absolute value) beyond those microRNA probes with p value equal or smaller than 0.01 by the Mann-Whitney test. This is, we used a so called ranker for feature selection. Again there are numerous other feature selection strategies we could have used, some examples are given in [M. A. Hall and G. Holmes. IEEE Transactions on Knowledge and Data Engineering, 15(6): 1437-1447, 2003.]. Overall a microRNA probe may have been chosen up to 100 times due to the 20 replications of the 5-fold cross-validation. We obtain the estimated errors given in Table 2.

TABLE 2
Table 2: microRNA data, classification error via 5-fold cross-validation

| classifier vs. true | NPMc− | NPMc+ |
|---|---|---|
| NPMc− | 57.0% | 7.6% |
| NPMc+ | 43.0% | 92.4% |

The estimated overall accuracy using 5-fold cross-validation is 79.9%. In a second step we now use only those microRNA arrays where there additionally is information about CD34 (i.e., CD34 negative or CD34 positive); selecting these samples 54 NPMc+ and 29 NPMC− samples remain. Using only CD34 for classification we obtain the results given in Table 3. which corresponds to an overall accuracy of 85.5%.

TABLE 3
Table 3: CD34 data, classification error

| classifier vs. true | NPMc− | NPMc+ |
|---|---|---|
| NPMc− | 75.9% | 9.3% |
| NPMc+ | 24.1% | 90.7% |

Now, if we combine the information of the top five microRNA probes with the CD34 information, we obtain the results given in Table 4. That is the estimated overall accuracy using cross-validation is 88.1%. Hence, this combination increases the overall accuracy from 79.9% respectively, 85.5% to 88.1%.

TABLE 4
Table 4: combination of microRNA and CD34, classification error
via 5-fold cross validation

| classifier vs. true | NPMc− | NPMc+ |
|---|---|---|
| NPMc− | 80.7% | 8.0% |
| NPMc+ | 19.3% | 92.0% |

The probes which were selected during cross-validation are given in Table 5.

TABLE 5
Table 5: microRNA probes selected during 5-fold cross validation

| | | Times | |
|---|---|---|---|
| Seq-ID | Probe ID | selected | Probe sequence |
| 1 | uc.124+ | 100 | TGCTCATCTGTGCACTTCTGTTCAACCTATCACACTGAGT |
| 2 | mmu-mir-335No2 | 97 | AAACCGTTTTTCATTATTGCTCCTGACCCCCTCTCATGGG |
| 3 | uc.368 + A | 96 | TGCACAGGGGACCTTAACCAGATCATTAGTTTATATGCCT |
| 4 | uc.324 + A | 93 | CACACACTCCAGAACAGATGGTATCCAGATGCCTTATGGG |
| 5 | uc.156+ | 74 | GCGAACCATTTCTAATGTTCTGATTTTTCAGAGCCAGCCA |
| 6 | hsa-mir-340No1 | 12 | TGTGGGATCCGTCTCAGTTACTTTATAGCCATACCTGGTA |
| 7 | uc.106+ | 6 | AGCTGAATGGTGATGGTGTGAAGTATAGGTTAAATTGGGT |
| 8 | hsa-mir-033b-prec | 4 | GTGCATTGCTGTTGCATTGCACGTGTGTGAGGCGGGTGCA |
| 9 | uc.54 + A | 4 | AAAGCTGTAGGGCCTCCAGGTTCTCAAGCTGTGAGTGGAA |
| 10 | uc.85+ | 4 | TGGTTGACATATGGCTGCTAATGCCCTCCTTTCTAGTGGG |
| 11 | uc.78 + A | 4 | GTGTGCGTAACGGCTGGTGTGTTTCTCTAGCTGAGCTAAT |
| 12 | mmu-mir-31No2 | 3 | ACCTGCTATGCCAACATATTGCCATCTTTCCTGTCTGACA |
| 13 | uc.195 + A | 2 | ACAGTGAGTGCGAGTATTATTTCTTGCCAGCGGGTGGAAG |
| 14 | uc.7 + A | 1 | ACACTGCTCGCTCTATGTTAATTTTAGCTCTTCCCCTGGA |

The results of the Sanger sequence search in accordance with Griffiths-Jones S, Saini H K, van Dongen S, Enright A J. miRBase: tools for microRNA genomics, NAR 2008 36 (Database Issue):D154-D158 for known human microRNAs are given in Table 6

TABLE 6
Table 6: Results of the Sanger sequence search for known human microRNAs
for the microRNA probes selected during 5-fold cross validation

| Seq-ID | Probe | microRNA ID | Target sequence |
|---|---|---|---|
| 15 | uc.124+ | hsa-mir-134 | CAGGGUGUGUGACUGGUUGACCAGAGGGGCAUGCAC |
| | | | UGUGUUCACCCUGUGGGCCACCUAGUCACCAACCCUC |
| 16 | mmu-mir-335No2 | hsa-mir-335 | UGUUUUGAGCGGGGGUCAAGAGCAAUAACGAAAAAUG |
| | | | UUUGUCAUAAACCGUUUUUCAUUAUUGCUCCUGACCU |
| | | | CCUCUCAUUUGCUAUAUUCA |
| 18 | hsa-mir-340No1 | hsa-mir-340 | UUGUACCUGGUGUGAUUAUAAAGCAAUGAGACUGAUU |
| | | | GUCAUAUGUCGUUUGUGGGAUCCGUCUCAGUUACUUU |
| | | | AUAGCCAUACCUGGUAUCUUA |
| 19 | uc.106+ | hsa-mir-138-1 | CCCUGGCAUGGUGUGGUGGGGCAGCUGGUGUUGUGA |
| | | | AUCAGGCCGUUGCCAAUCAGAGAACGGCUACUUCACA |
| | | | ACACCAGGGCCACACCACACUACAGG |
| 20 | hsa-mir-033b-prec | hsa-mir-33b | GCGGGCGGCCCCGCGGUGCAUUGCUGUUGCAUUGCA |
| | | | CGUGUGUGAGGCGGGUGCAGUGCCUCGGCAGUGCAG |
| | | | CCCGGAGCCGGCCCCUGGCACCAC |
| 21 | uc.54 + A | hsa-mir-339 | CGGGGCGGCCGCUCUCCCUGUCCUCCAGGAGCUCAC |
| | | | GUGUGCCUGCCUGUGAGCGCCUCGACGACAGAGCCG |
| | | | GCGCCUGCCCCAGUGUCUGCGC |
| 22 | uc.85+ | hsa-mir-1976 | GCAGCAAGGAAGGCAGGGGUCCUAAGGUGUGUCCUCC |
| | | | UGCCCUCCUUGCUGU |
| 23 | uc.78 + A | hsa-mir-223 | CCUGGCCUCCUGCAGUGCCACGCUCCGUGUAUUUGAC |
| | | | AAGCUGAGUUGGACACUCCAUGUGGUAGAGUGUCAGU |
| | | | UUGUCAAAUACCCCAAGUGCGGCACAUGCUUACCAG |
| 24 | mmu-mir-31 No2 | hsa-mir-31 | GGAGAGGAGGCAAGAUGCUGGCAUAGCUGUUGAACUG |
| | | | GGAACCUGCUAUGCCAACAUAUUGCCAUCUUUCC |
| 25 | uc.195 + A | hsa-mir-548a- | CCUAGAAUGUUAUUAGGUCGGUGCAAAAGUAAUUGCG |
| | | 3 | AGUUUUACCAUUACUUUCAAUGGCAAAACUGGCAAUUA |
| | | | CUUUUGCACCAACGUAAUACUU |
| 26 | uc.7 + A | hsa-mir-1912 | CUCUAGGAUGUGCUCAUUGCAUGGGCUGUGUAUAGUA |
| | | | UUAUUCAAUACCCAGAGCAUGCAGUGUGAACAUAAUAG |
| | | | AGAUU |

### Example 2.1

### mRNA and microRNA: Colon Cancer

We use the colon cancer data of Ramaswamy et al. (2001) [Ramaswamy S, Tamayo P, Rifkin R, Mukherjee S, Yeang C H, Angelo M, Ladd C, Reich M, Latulippe E, Mesirov J P, Poggio T, Gerald W, Loda M, Lander E S, Golub T R. Multiclass cancer diagnosis using tumor gene expression signatures. Proc Natl Acad Sci USA. 2001; 98(26):15149-54] and Lu et al. (2005) [Lu J, Getz G, Miska E A, Alvarez-Saavedra E, Lamb J, Peck D, Sweet-Cordero A, Ebert B L, Mak R H, Ferrando A A, Downing J R, Jacks T, Horvitz H R, Golub T R. MicroRNA expression profiles classify human cancers. Nature. 2005; 435(7043):834-8] to develop a multilevel classifier using mRNA and microRNA data. The data are available from the home page of the Broad Institute [http://www.broad.mit.edu/publications/broad900 and http://www.broad.mit.edu/publications/broad993s].

Overall the mRNA and microRNA data of four normal tissues and seven tumor tissues are available. The hybridisations were done with a bead-based array containing microRNA probes as well as with the Affymetrix HU6800 and HU35KsubA array for measuring the mRNA. We used only the mRNA data of the HU6800 arrays.

Analysis:

For developing and validating a classifier based on these data we used random forests [Breiman, L Random Forests, Machine Learning 2001, 45(1), 5-32] in combination with leave-one-out (LOO) cross-validation where each analysis step—including low level analysis—was repeated in each cross-validation step. This is one possibility. Of course, we could also have used a split-sample, a bootstrap or a different k-fold (k not equal to 1) cross-validation approach. Moreover, we could have used a different class of functions for classification e.g. logistic regression, (diagonal) linear or quadratic discriminant analysis (LDA, QDA, DLDA, DQDA), shrunken centroids regularized discriminant analysis (RDA), neural networks (NN), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more.

The preprocessing (also called low level analysis) consisted of the variance stabilizing transformation of Huber et al (2002) (often called normalization) in case of the microRNA as well as of the mRNA data. Again there is a large number of alternative methods which could be used Several examples are given in Cope et al. (2004) or Irizarry et al. (2006). In each cross validation step we selected those six normalized microRNA probes, respectively those six normalized mRNA probes for classification which had the largest median of pairwise differences (in absolute value) beyond those probes with p value equal or smaller than 0.1 by the Mann-Whitney test. This is we used a so called ranker for feature selection. Again there are numerous other feature selection strategies we could have used some examples are given in [M. A. Hall and G. Holmes. IEEE Transactions on Knowledge and Data Engineering, 15(6): 1437-1447, 2003.]. Overall a microRNA, respectively mRNA probe may have been chosen up to eleven times due to LOO cross-validation.

Using only microRNA data we obtain the estimated errors given in Table 7

TABLE 7
Table 7: microRNA data, classification error via leave-one-out
cross validation

| classifier vs. true | colon cancer | normal |
|---|---|---|
| colon cancer | 85.7% | 0.0% |
| normal | 14.3% | 100.0% |

That is, we observe a sensitivity of 85.7% and a specificity of 100.0%. The positive predictive value is equal to 100.0%, the negative predictive value is equal to 80%. The estimated overall accuracy using LOO cross-validation is 90.9%. In a second step we used the mRNA data of the HU6800 array. The results can be read off from Table 8. We get an estimated overall accuracy of 72.7% again using LOO cross-validation. The estimated sensitivity is equal to 85.7%, the estimated specificity is equal to 50%, the estimated positive predictive value is equal to 75.0%, the estimated negative predictive value is equal to 66.7%.

TABLE 8
Table 8: mRNA data, classification error via leave-one-out
cross validation

| classifier vs. true | colon cancer | normal |
|---|---|---|
| colon cancer | 85.7% | 50.0% |
| normal | 14.3% | 50.0% |

In the last step we combined microRNA and mRNA data and obtained the results given in Table 9. That is, the estimated overall accuracy using cross-validation is 100.0%. Hence, this combination increases the overall accuracy from 90.9% respectively, 72.7% to 100.0%. Likewise sensitivity, specificity, positive predictive value and negative predictive value increase to 100%.

TABLE 9
Table 9: microRNA and mRNA data, classification error via
leave-one-out cross validation

| classifier vs. true | colon cancer | normal |
|---|---|---|
| colon cancer | 100.0% | 0.0% |
| normal | 0.0% | 100.0% |

The microRNA probes which were selected during cross-validation are given in Table 10.

TABLE 10
Table 10: microRNA probes selected during
leave-one-out cross validation

| Seq- | | Times | |
|---|---|---|---|
| ID | Probe ID | selected | Probe sequence |
| 27 | hsa-miR-1 | 11 | ATACATACTTCTTTACATTCCA |
| 28 | mmu-miR-10b | 11 | ACACAAATTCGGTTCTACAGGG |
| 29 | hsa-miR-195 | 11 | GCCAATATTTCTGTGCTGCTA |
| 30 | hsa-miR- | 11 | ACAGCTGGTTGAAGGGGACCAA |
| | 133a | | |
| 31 | hsa-miR- | 10 | CACATAGGAATGAAAAGCCATA |
| | 135b | | |
| 32 | hsa-miR-182 | 7 | TGTGAGTTCTACCATTGCCAAA |
| 33 | hsa-miR-30e | 4 | TCCAGTCAAGGATGTTTACA |
| 34 | hsa-miR-99a | 1 | CACAAGATCGGATCTACGGGT |

The results of the Sanger sequence search (see Griffiths-Jones S, Saini H K, van Dongen S, Enright A J. miRBase: tools for microRNA genomics. NAR 2008 36 (Database Issue):D154-D158) for known human microRNAs are given in Table 11

TABLE 11
Table 11: Results of the Sanger sequence search for known human
microRNAs or the microRNA probes selected during 5-fold cross validation

| Seq-ID | Probe ID | microRNA ID | Target sequence |
|---|---|---|---|
| 35 | hsa-miR-1 | hsa-mir-1 | ACCUACUCAGAGUACAUACUUCUUUAUGUACCCAUAUGAA |
| | | | CAUACAAUGCUAUGGAAUGUAAAGAAGUAUGUAUUUUUGG |
| | | | UAGGC |
| 36 | mmu-miR-10b | hsa-mir-10b | CCAGAGGUUGUAACGUUGUCUAUAUAUACCCUGUAGAACC |
| | | | GAAUUUGUGUGGUAUCCGUAUAGUCACAGAUUCGAUUCUA |
| | | | GGGGAAUAUAUGGUCGAUGCAAAAACUUCA |
| 37 | hsa-miR-195 | hsa-mir-195 | AGCUUCCCUGGCUCUAGCAGCACAGAAAUAUUGGCACAGG |
| | | | GAAGCGAGUCUGCCAAUAUUGGCUGUGCUGCUCCAGGCA |
| | | | GGGUGGUG |
| 38 | hsa-miR-133a | hsa-mir- | ACAAUGCUUUGCUAGAGCUGGUAAAAUGGAACCAAAUCGC |
| | | 133a | CUCUUCAAUGGAUUUGGUCCCCUUCAACCAGCUGUAGCUA |
| | | | UGCAUUGA |
| 39 | hsa-miR-135b | hsa-mir- | CACUCUGCUGUGGCCUAUGGCUUUUCAUUCCUAUGUGAU |
| | | 135b | UGCUGUCCCAAACUCAUGUAGGGCUAAAAGCCAUGGGCUA |
| | | | CAGUGAGGGGCGAGCUCC |
| 40 | hsa-miR-182 | hsa-mir-182 | GAGCUGCUUGCCUCCCCCCGUUUUUGGCAAUGGUAGAAC |
| | | | UCACACUGGUGAGGUAACAGGAUCCGGUGGUUCUAGACU |
| | | | UGCCAACUAUGGGGCGAGGACUCAGCCGGCAC |
| 41 | hsa-miR-30e | hsa-mir-30e | GGGCAGUCUUUGCUACUGUAAACAUCCUUGACUGGAAGCU |
| | | | GUAAGGUGUUCAGAGGAGCUUUCAGUCGGAUGUUUACAG |
| | | | CGGCAGGCUGCCA |
| 42 | hsa-miR-99a | hsa-mir-99a | CCCAUUGGCAUAAACCCGUAGAUCCGAUCUUGUGGUGAAG |
| | | | UGGACCGCACAAGCUCGCUUCUAUGGGUCUGUGUCAGUG |
| | | | UG |

The mRNA probes which were selected during cross-validation are given in Table 12. The probe sequences were obtained from Bioconductor package hu6800probe [The Bioconductor Project, www.bioconductor.org (2008). hu6800probe: Probe sequence data for microarrays of type hu6800. R package version 2.2.01

TABLE 12
Table 12: mRNA probes selected during leave-one-out cross validation

| | | Times | |
|---|---|---|---|
| Seq-ID | Affymetrix ID | selected | Probe Sequences (Perfect Match) |
| 43-62 | AFFX- | 11 | [1] AAGATCATTGCTCCTCCTGAGCGCA |
| | HSAC07/X00351_M_at | | [2] CCTCCTGAGCGCAAGTACTCCGTGT |
| | | | [3] TCCGTGTGGATCGGCGGCTCCATCC |
| | | | [4] CAGATGTGGATCAGCAAGCAGGAGT |
| | | | [5] GTCCACCGCAAATGCTTCTAGGCGG |
| | | | [6] ACCACGGCCGAGCGGGAAATCGTGC |
| | | | [7] CTGTGCTACGTCGCCCTGGACTTCG |
| | | | [8] GAGCAAGAGATGGCCACGGCTGCTT |
| | | | [9] TCCTCCCTGGAGAAGAGCTACGAGC |
| | | | [10] CTGCCTGACGGCCAGGTCATCACCA |
| | | | [11] CAGGTCATCACCATTGGCAATGAGC |
| | | | [12] CGGTTCCGCTGCCCTGAGGCACTCT |
| | | | [13] CCTGAGGCACTCTTCCAGCCTTCCT |
| | | | [14] GAGTCCTGTGGCATCCACGAAACTA |
| | | | [15] ATCCACGAAACTACCTTCAACTCCA |
| | | | [16] AACTCCATCATGAAGTGTGACGTGG |
| | | | [17] GACATCCGCAAAGACCTGTACGCCA |
| | | | [18] AACACAGTGCTGTCTGGCGGCACCA |
| | | | [19] ACCATGTACCCTGGCATTGCCGACA |
| | | | [20] CAGAAGGAGATCACTGCCCTGGCAC |
| 63-81 | X03689_s_at | 10 | [1] AGATTCGGGCAAGTCCACCACTACT |
| | | | [2] TTCGGGCAAGTCCACCACTACTGGC |
| | | | [3] CACCACTACTGGCCATCTGATCTAT |
| | | | [4] CCATCTGATCTATAAATGCGGTGGC |
| | | | [5] TCTGATCTATAAATGCGGTGGCATC |
| | | | [6] TGCCTGGGTCTTGGATAAACTGAAA |
| | | | [7] TGAAAGCTGAGCGTGAACGTGGTAT |
| | | | [8] CGTGAACGTGGTATCACCATTGATA |
| | | | [9] GAACGTGGTATCACCATTGATATCT |
| | | | [10] GTGGTATCACCATTGATATCTCCTT |
| | | | [11] TATCACCATTGATATCTCCTTGTGG |
| | | | [12] CCATTGATATCTCCTTGTGGAAATT |
| | | | [13] GTACTATGTGACTATCATTGATGCC |
| | | | [14] CTATGTGACTATCATTGATGCCCCA |
| | | | [15] CTCATATCAACATTGTCGTCATTGG |
| | | | [16] TATCAACATTGTCGTCATTGGACAC |
| | | | [17] CATTGTCGTCATTGGACACGTAGAT |
| | | | [18] TGTCGTCATTGGACACGTAGATTCG |
| | | | [19] CGTCATTGGACACGTAGATTCGGGC |
| 82-101 | AFFX- | 9 | [1] GGGTCAGAAGGATTCCTATGTGGGC |
| | HSAC07/X00351_5_at | | [2] GAAGGATTCCTATGTGGGCGACGAG |
| | | | [3] CCCCATCGAGCACGGCATCGTCACC |
| | | | [4] CGTCACCAACTGGGACGACATGGAG |
| | | | [5] CACCTTCTACAATGAGCTGCGTGTG |
| | | | [6] TCCCGAGGAGCACCCCGTGCTGCTG |
| | | | [7] GGCCAACCGCGAGAAGATGACCCAG |
| | | | [8] CCAGATCATGTTTGAGACCTTCAAC |
| | | | [9] CCCAGCCATGTACGTTGCTATCCAG |
| | | | [10] CGTTGCTATCCAGGCTGTGCTATCC |
| | | | [11] GGCTGTGCTATCCCTGTACGCCTCT |
| | | | [12] CGCCTCTGGCCGTACCACTGGCATC |
| | | | [13] TACCACTGGCATCGTGATGGACTCC |
| | | | [14] CGGTGACGGGGTCACCCACACTGTG |
| | | | [15] CCACACTGTGCCCATCTACGAGGGG |
| | | | [16] GCCCATCTACGAGGGGTATGCCCTC |
| | | | [17] TGCCATCCTGCGTCTGGACCTGGCT |
| | | | [18] TGATATCGCCGCGCTCGTCGTCGAC |
| | | | [19] CGTCGTCGACAACGGCTCCGGCATG |
| | | | [20] CGGCTCCGGCATGTGCAAGGCCGGC |
| 102-121 | M18728_at | 8 | [1] ACCCTCCTAATAGTCATACTAGTAG |
| | | | [2] CTAATAGTCATACTAGTAGTCATAC |
| | | | [3] GTCATACTAGTAGTCATACTCCCTG |
| | | | [4] CTAGTAGTCATACTCCCTGGTGTAG |
| | | | [5] ATGCAGCCAGCCATCAAATAGTGAA |
| | | | [6] TAGTGAATGGTCTCTCTTTGGCTGG |
| | | | [7] TAACCCATGAAGGATAAAAGCCCCA |
| | | | [8] ATAGCACTAATGCTTTAAGATTTGG |
| | | | [9] CTTTAAGATTTGGTCACACTCTCAC |
| | | | [10] GATTTGGTCACACTCTCACCTAGGT |
| | | | [11] CATTGAGCCAGTGGTGCTAAATGCT |
| | | | [12] GGTGCTAAATGCTACATACTCCAAC |
| | | | [13] TACATACTCCAACTGAAATGTTAAG |
| | | | [14] CTCCAACTGAAATGTTAAGGAAGAA |
| | | | [15] AACACAGGAGATTCCAGTCTACTTG |
| | | | [16] GCATAATACAGAAGTCCCCTCTACT |
| | | | [17] GTAACCTGAACTAATCTGATGTTAA |
| | | | [18] AATCTGATGTTAACCAATGTATTTA |
| | | | [19] CTGTTTCCTTGTTCCAATTTGACAA |
| | | | [20] GCTATCACTGTACTTGTAGAGTGGT |
| 122-141 | AFFX- | 7 | [1] GCGCCTGGTCACCAGGGCTGCTTTT |
| | HUMGAPDH/M33197_5_at | | [2] GGTCACCAGGGCTGCTTTTAACTCT |
| | | | [3] TGCTTTTAACTCTGGTAAAGTGGAT |
| | | | [4] GGATATTGTTGCCATCAATGACCCC |
| | | | [5] CATCAATGACCCCTTCATTGACCTC |
| | | | [6] CTTCATTGACCTCAACTACATGGTT |
| | | | [7] CAACTACATGGTTTACATGTTCCAA |
| | | | [8] GGTTTACATGTTCCAATATGATTCC |
| | | | [9] CCAATATGATTCCACCCATGGCAAA |
| | | | [10] TGATTCCACCCATGGCAAATTCCAT |
| | | | [11] ATTCCATGGCACCGTCAAGGCTGAG |
| | | | [12] TGGCACCGTCAAGGCTGAGAACGGG |
| | | | [13] CATCAATGGAAATCCCATCACCATC |
| | | | [14] TCCCATCACCATCTTCCAGGAGCGA |
| | | | [15] CTTCCAGGAGCGAGATCCCTCCAAA |
| | | | [16] GCGAGATCCCTCCAAAATCAAGTGG |
| | | | [17] CGATGCTGGCGCTGAGTACGTCGTG |
| | | | [18] CGTGGAGTCCACTGGCGTCTTCACC |
| | | | [19] CTTCACCACCATGGAGAAGGCTGGG |
| | | | [20] CGGATTTGGTCGTATTGGGCGCCTG |
| 142-161 | X00351_f_at | 6 | [1] TCCTCCTGAGCGCAAGTACTCCGTG |
| | | | [2] TGAGCGCAAGTACTCCGTGTGGATC |
| | | | [3] CTTCCAGCAGATGTGGATCAGCAAG |
| | | | [4] GTGGATCAGCAAGCAGGAGTATGAC |
| | | | [5] CCGCAAATGCTTCTAGGCGGACTAT |
| | | | [6] ATGCTTCTAGGCGGACTATGACTTA |
| | | | [7] TAACTTGCGCAGAAAACAAGATGAG |
| | | | [8] CAGCAGTCGGTTGGAGCGAGCATCC |
| | | | [9] CAATGTGGCCGAGGACTTTGATTGC |
| | | | [10] GGCCGAGGACTTTGATTGCACATTG |
| | | | [11] TGACGTGGACATCCGCAAAGACCTG |
| | | | [12] GTACGCCAACACAGTGCTGTCTGGC |
| | | | [13] CAACACAGTGCTGTCTGGCGGCACC |
| | | | [14] GTCTGGCGGCACCACCATGTACCCT |
| | | | [15] CACCATGTACCCTGGCATTGCCGAC |
| | | | [16] GTACCCTGGCATTGCCGACAGGATG |
| | | | [17] TGCCGACAGGATGCAGAAGGAGATC |
| | | | [18] GGAGATCACTGCCCTGGCACCCAGC |
| | | | [19] CCTGGCACCCAGCACAATGAAGATC |
| | | | [20] ACCCAGCACAATGAAGATCAAGATC |
| 162-181 | M77349_at | 5 | [1] TGAAGCACTACAGGAGGAATGCACC |
| | | | [2] AGCTCTCCGCCAATTTCTCTCAGAT |
| | | | [3] AATGTACATGGGCCGCACCATAATG |
| | | | [4] CATGGGCCGCACCATAATGAGATGT |
| | | | [5] CCGCACCATAATGAGATGTGAGCCT |
| | | | [6] TGGCTGTTAACCCACTGCATGCAGA |
| | | | [7] TTAACCCACTGCATGCAGAAACTTG |
| | | | [8] CACTGCATGCAGAAACTTGGATGTC |
| | | | [9] TGGAATTGACTGCCTATGCCAAGTC |
| | | | [10] TGACTGCCTATGCCAAGTCCCTGGA |
| | | | [11] CTCATAAAACATGAATCAAGCAATC |
| | | | [12] GAATCAAGCAATCCAGCCTCATGGG |
| | | | [13] TTGTAAAGCCCTTGCACAGCTGGAG |
| | | | [14] TGCACAGCTGGAGAAATGGCATCAT |
| | | | [15] GCATCATTATAAGCTATGAGTTGAA |
| | | | [16] AATGTTCTGTCAAATGTGTCTCACA |
| | | | [17] AATGTGTCTCACATCTACACGTGGC |
| | | | [18] TCTCACATCTACACGTGGCTTGGAG |
| | | | [19] TTCCCTATTGTGACAGAGCCATGGT |
| | | | [20] ATTGTGACAGAGCCATGGTGTGTTT |
| 182-192 | M34516_r_at | 3 | [1] TTCTCCCTGCACTCATGAAACCCCA |
| | | | [2] TCTCCCTGCACTCATGAAACCCCAA |
| | | | [3] GCACTCATGAAACCCCAATAAATAT |
| | | | [4] CACTCATGAAACCCCAATAAATATC |
| | | | [5] ACTCATGAAACCCCAATAAATATCC |
| | | | [6] CTCATGAAACCCCAATAAATATCCT |
| | | | [7] TCATGAAACCCCAATAAATATCCTC |
| | | | [8] CATGAAACCCCAATAAATATCCTCA |
| | | | [9] ATGAAACCCCAATAAATATCCTCAT |
| | | | [10] AAACCCCAATAAATATCCTCATTGA |
| | | | [11] AACCCCAATAAATATCCTCATTGAC |
| 193-199 | D49824_s_at | 2 | [1] GGCTGTCCTAGCAGTTGTGGTCATC |
| | | | [2] CTGTCCTAGCAGTTGTGGTCATCGG |
| | | | [3] TGTCCTAGCAGTTGTGGTCATCGGA |
| | | | [4] GTCCTAGCAGTTGTGGTCATCGGAG |
| | | | [5] TCCTAGCAGTTGTGGTCATCGGAGC |
| | | | [6] CTAGCAGTTGTGGTCATCGGAGCTG |
| | | | [7] TAGCAGTTGTGGTCATCGGAGCTGT |
| 220-239 | J03040_at | 1 | [1] GGTTTGCCTGAGGCTGTAACTGAGA |
| | | | [2] CCTGAGGCTGTAACTGAGAGAAAGA |
| | | | [3] ATTCTGGGGCTGTCTTATGAAAATA |
| | | | [4] ATAGACATTCTCACATAAGCCCAGT |
| | | | [5] ACATAAGCCCAGTTCATCACCATTT |
| | | | [6] TCACATTAGGCTGTTGGTTCAAACT |
| | | | [7] GAGCACGGACTGTCAGTTCTCTGGG |
| | | | [8] GGACTGTCAGTTCTCTGGGAAGTGG |
| | | | [9] GAAGTGGTCAGCGCATCCTGCAGGG |
| | | | [10] GTCAGCGCATCCTGCAGGGCTTCTC |
| | | | [11] TTTGGAGAACCAGGGCTCTTCTCAG |
| | | | [12] GAACCAGGGCTCTTCTCAGGGGCTC |
| | | | [13] TTCTCAGGGGCTCTAGGGACTGCCA |
| | | | [14] CTAGGGACTGCCAGGCTGTTTCAGC |
| | | | [15] TTTCAGCCAGGAAGGCCAAAATCAA |
| | | | [16] GGGATGGTCGGATCTCACAGGCTGA |
| | | | [17] GTCGGATCTCACAGGCTGAGAACTC |
| | | | [18] TCTCACAGGCTGAGAACTCGTTCAC |
| | | | [19] CCTCCAAGCATTTCATGAAAAAGCT |
| | | | [20] AGCATTTCATGAAAAAGCTGCTTCT |
| 240-259 | M13560_s_at | 1 | [1] CAGGATCTGGGCCCAGTCCCCATGT |
| | | | [2] GGCCCAGTCCCCATGTGAGAGCAGC |
| | | | [3] CCCATGTGAGAGCAGCAGAGGCGGT |
| | | | [4] AGAGCAGCAGAGGCGGTCTTCAACA |
| | | | [5] ACACAGCTACAGCTTTCTTGCTCCC |
| | | | [6] CAAGACAAACCAAGTCGGAACAGCA |
| | | | [7] CAAGTCGGAACAGCAGATAACAATG |
| | | | [8] TGCCCAATCTCCATCTGTCAACAGG |
| | | | [9] TGAGGTCCCAGGAAGTGGCCAAAAG |
| | | | [10] AGCTAGACAGATCCCCGTTCCTGAC |
| | | | [11] GACATCACAGCAGCCTCCAACACAA |
| | | | [12] CAACACAAGGCTCCAAGACCTAGGC |
| | | | [13] AAGACCTAGGCTCATGGACGAGATG |
| | | | [14] CCAGACCCCAGGCTGGACATGCTGA |
| | | | [15] CCTTTGGCCTTGGCTTTTCTAGCCT |
| | | | [16] TTGGCTTTTCTAGCCTATTTACCTG |
| | | | [17] AGCCTATTTACCTGCAGGCTGAGCC |
| | | | [18] GCTCAGCCAAGCTTGTTATCAGCTT |
| | | | [19] AAGCTTGTTATCAGCTTTCAGGGCC |
| | | | [20] ATCAGCTTTCAGGGCCATGGTTCAC |
| 260-264 | M34516_at | 1 | [1] TCCCTGCACTCATGAAACCCCAATA |
| | | | [2] CCCTGCACTCATGAAACCCCAATAA |
| | | | [3] CCTGCACTCATGAAACCCCAATAAA |
| | | | [4] CTGCACTCATGAAACCCCAATAAAT |
| | | | [5] TGCACTCATGAAACCCCAATAAATA |

Miss match (MM) probes are obtained by altering the medium amino acid, more precise A becomes T, T becomes A, G becomes C and C becomes G. The probe sequences each have a length of 25, i.e. the respective 13. amino acids are replaced.

The annotations of the selected mRNA probes are given in Table 13. The annotations were obtained from Bioconductor package hu6800.db [Marc Carlson, Seth Falcon, Herve Pages and Nianhua Li (2008). hu6800.db: Affymetrix HuGeneFL Genome Array annotation data (chip hu6800). R package version 2.2.3.] in combination with the information available via PubMed [http://www.ncbi.nlm.nih.gov/pubmed/].

TABLE 13
Annotation of mRNA probes selected during LOO cross validation

| | | Accession | | |
|---|---|---|---|---|
| Seq-ID | Affymetrix ID | number | RefSeq ID | Unigene ID |
| 265 | AFFX-HSAC07/X00351_M_at | X00351 | NM_001101.2 | Hs.520640 |
| | | | | Hs.708120 |
| 266 | X03689_s_at | X03689 | NM_001402.2 | Hs.520703 |
| | | | | Hs.586423 |
| | | | | Hs.644639 |
| | | | | Hs.703481 |
| | | | | Hs.708256 |
| 265 | AFFX-HSAC07/X00351_5_at | X00351 | NM_001101.2 | Hs.520640 |
| | | | | Hs.708120 |
| 267 | M18728_at | M18728 | NM_002483.3 | Hs.466814 |
| 268 | AFFX- | M33197 | NM_002046.3 | Hs.544577 |
| | HUMGAPDH/M33197_5_at | | | Hs.592355 |
| | | | | Hs.711936 |
| 265 | X00351_f_at | X00351 | NM_001101.2 | Hs.520640 |
| | | | | Hs.708120 |
| 269 | M77349_at | M77349 | NM_000358.1 | Hs.369397 |
| | | | | Hs.645734 |
| 270 | M34516_r_at | M34516 | NM_001013618.1 | Hs.449585 |
| 271 | D49824_s_at | D49824 | NM_005514.5 | Hs.77961 |
| | | | | Hs.703277 |
| | | | | Hs.707171 |
| 272 | D00654_at | D00654 | NM_001615.3 | Hs.516105 |
| 273 | HG3044-HT3742_s_at | HG3044-HT3742 | NM_212482.1 | Hs.203717 |
| 274 | J03040_at | J03040 | NM_003118.2 | Hs.111779 |
| | | | | Hs.708558 |
| 275 | M13560_s_at | M13560 | NM_001025159.1 | Hs.436568 |
| 276 | M34516_at | M34516 | NM_020070.2 | Hs.348935 |

### Example 2.2

### mRNA and microRNA: Kidney Cancer

We use the kidney cancer data of Ramaswamy et al. (2001) [Ramaswamy S, Tamayo P, Rifkin R, Mukherjee S, Yeang C H, Angelo M, Ladd C, Reich M, Latulippe E, Mesirov J P, Poggio T, Gerald W, Loda M, Lander E S, Golub T R. Multiclass cancer diagnosis using tumor gene expression signatures. Proc Natl Acad Sci USA. 2001; 98(26):15149-54] and Lu et al. (2005) [Lu J, Getz G, Miska E A, Alvarez-Saavedra E, Lamb J, Peck D, Sweet-Cordero A, Ebert B L, Mak R H, Ferrando A A, Downing J R, Jacks T, Horvitz H R, Golub T R. MicroRNA expression profiles classify human cancers. Nature. 2005; 435(7043):834-8] to develop a multilevel classifier using mRNA and microRNA data. The data are available from the home page of the Braoad Institute [see http://www.broad.mit.edu/publications/broad900 and http://www.broad.mit.edu/publications/broad993s]. Overall the mRNA and microRNA data of three normal tissues and four tumor tissues are available. The hybridisations were done with a bead-based array containing microRNA probes as well as with the Affymetrix HU6800 and HU35KsubA array for measuring the mRNA. We used only the mRNA data of the HU35KsubA arrays.

Analysis:

For developing and validating a classifier based on these data we used single-hidden-layer neural networks [Ripley, B. D. (1996) Pattern Recognition and Neural Networks. Cambridge] in combination with leave-one-out (LOO) cross-validation where each analysis step—including low level analysis—was repeated in each cross-validation step. This is one possibility. Of course, we could also have used a split-sample, a bootstrap or a different k-fold (k not equal to 1) cross-validation approach. Moreover, we could have used a different class of functions for classification e.g. logistic regression, (diagonal) linear or quadratic discriminant analysis (LDA, QDA, DLDA, DQDA), shrunken centroids regularized discriminant analysis (RDA), random forests (RF), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more.

The low level analysis (preprocessing) consisted of the variance stabilizing transformation of Huber et al (2002) (often called normalization) in case of the microRNA as well as of the mRNA data. Again there is a large number of alternative methods which could be used. Several examples are given in Cope et al. (2004) or Irizarry et al. (2006) In each cross validation step we selected those six normalized microRNA probes, respectively those six normalized mRNA probes for classification which had the largest differences (in absolute value) of the mean values beyond those probes with p value equal or smaller than 0.1 by the Welch t-test. This is, we used a so called ranker for feature selection. Again there are numerous other feature selection strategies we could have used, some examples are given in Hall et al. (2003). Overall a microRNA, respectively mRNA probe may have been chosen up to seven times due to LOO cross-validation.

Using only microRNA data we obtain the estimated errors given in Table 14

TABLE 14
Table 14: microRNA data, classification error via LOO cross validation

| classifier vs. true | kidney cancer | Normal |
|---|---|---|
| kidney cancer | 50.0% | 66.7% |
| normal | 50.0% | 33.3% |

The estimated overall accuracy using LOO cross-validation is 42.9%, sensitivity is 50%, specificity is 33.3%, positive predictive value is 50% and negative predictive value is 33.3%. In a second step we used the mRNA data of the HU35KsubA array. The results can be read off from Table 15. We get an estimated overall accuracy of 42.9% again using LOO cross-validation. The estimated values for sensitivity, specificity, positive and negative predictive value are 50%, 33.3%, 50% and 33.3%, respectively.

TABLE 15
Table 15: mRNA data, classification error via LOO cross validation

| classifier vs. true | kidney cancer | Normal |
|---|---|---|
| kidney cancer | 50.0% | 66.7% |
| normal | 50.0% | 33.3% |

In the last step we combine microRNA and mRNA data and obtain the results given in Table 16. That is, the estimated overall accuracy using cross-validation is 71.4%. Hence, this combination increases the overall accuracy from 42.9% to 71.4%. Sensitivity, specificity, positive and negative predictive value are increased to 75.0%, 66.7%, 75.0% and 66.7%, respectively.

TABLE 16
Table 16: microRNA and mRNA data, classification error via
LOO cross validation

| classifier vs. true | kidney cancer | Normal |
|---|---|---|
| kidney cancer | 75.0% | 33.3% |
| normal | 25.0% | 66.7% |

The microRNA probes which were selected during cross-validation are given in Table 17.

TABLE 17
Table 17: microRNA probes selected during LOO
cross validation (1^{st} column is SEQ-ID-No)

| Seq- | | Times | |
|---|---|---|---|
| ID | Probe ID | selected | Probe sequence |
| 277 | pre- | 5 + 5 + | CTGACTGACTGACTGACTGACTG |
| | control 3 | 5* | |
| 278 | pre- | 5 + 1 | TTGTACGTTTACATGGAGGTC |
| | control 4 | | |
| 279 | hsa-let-7b | 4 | AACCACACAACCTACTACCTCA |
| 280 | FVR506 | 4 + 1 | TGTATTCCTCGCCTGTCCAG |
| 281 | hsa-miR-320 | 2 | TCGCCCTCTCAACCCAGCTTTT |
| 282 | hsa-let-7a | 2 | AACTATACAACCTACTACCTCA |
| 283 | hsa-let-7c | 1 | AACCATACAACCTACTACCTCA |
| 284 | hsa-miR-30b | 1 | GCTGAGTGTAGGATGTTTACA |
| 285 | has-miR-10a | 1 | ACACAAATTCGGTTCTACAGGG |
| 286 | PTG20210 | 1 + 1 | CATTGAGGCTCGCTGAGAGT |
| 33 | hsa-miR-30e | 1 | TCCAGTCAAGGATGTTTACA |
| 287 | hsa-miR-339 | 1 | TGAGCTCCTGGAGGACAGGGA |
| 288 | pre- | 1 | CTTGTACCAGTTATCTGCAA |
| | control 5 | | |

*Some probes occur in replicates

The results of the Sanger sequence search in accordance with Griffiths-Jones et al. 2008 for known human microRNAs are given in Table 18

TABLE 18
Table 18: Results of the Sanger sequence search for known human
microRNAs for microRNA probes selected during LOO cross validation
(1^{st} column is SEQ-ID-No)

| | | | |
|---|---|---|---|
| | pre-control 3 | | |
| 289 | pre-control 4 | hsa-mir-302d | CCUCUACUUUAACAUGGAGGCACUUGCUGUGACAU |
| | | | GACAAAAAUAAGUGCUUCCAUGUUUGAGUGUGG |
| 290 | hsa-let-7b | hsa-let-7b | CGGGGUGAGGUAGUAGGUUGUGUGGUUUCAGGGCA |
| | | | GUGAUGUUGCCCCUCGGAAGAUAACUAUACAACCUA |
| | | | CUGCCUUCCCUG |
| 291 | FVR506 | hsa-mir-1238 | GUGAGUGGGAGCCCCAGUGUGUGGUUGGGGCCAU |
| | | | GGCGGGUGGGCAGCCCAGCCUCUGAGCCUUCCUCG |
| | | | UCUGUCUGCCCCAG |
| 292 | hsa-miR-320 | hsa-mir-320a | GCUUCGCUCCCCUCCGCCUUCUCUUCCCGGUUCUU |
| | | | CCCGGAGUCGGGAAAAGCUGGGUUGAGAGGGCGAA |
| | | | AAAGGAUGAGGU |
| 293 | hsa-let-7a | hsa-let-7a | UGGGAUGAGGUAGUAGGUUGUAUAGUUUUAGGGUC |
| | | | ACACCCACCACUGGGAGAUAACUAUACAAUCUACUG |
| | | | UCUUUCCUA |
| 294 | hsa-let-7c | hsa-let-7c | GCAUCCGGGUUGAGGUAGUAGGUUGUAUGGUUUAG |
| | | | AGUUACACCCUGGGAGUUAACUGUACAACCUUCUAG |
| | | | CUUUCCUUGGAGC |
| 295 | hsa-miR-30b | hsa-mir-30b | ACCAAGUUUCAGUUCAUGUAAACAUCCUACACUCAG |
| | | | CUGUAAUACAUGGAUUGGCUGGGAGGUGGAUGUUU |
| | | | ACUUCAGCUGACUUGGA |
| | PTG20210 | | |
| 41 | hsa-miR-30e | hsa-mir-30e | GGGCAGUCUUUGCUACUGUAAACAUCCUUGACUGG |
| | | | AAGCUGUAAGGUGUUCAGAGGAGCUUUCAGUCGGA |
| | | | UGUUUACAGCGGCAGGCUGCCA |
| 21 | hsa-miR-339 | hsa-mir-339 | CGGGGCGGCCGCUCUCCCUGUCCUCCAGGAGCUCA |
| | | | CGUGUGCCUGCCUGUGAGCGCCUCGACGACAGAGC |
| | | | CGGCGCCUGCCCCAGUGUCUGCGC |
| 297 | pre-control 5 | hsa-mir-150 | CUCCCCAUGGCCCUGUCUCCCAACCCUUGUACCAG |
| | | | UGCUGGGCUCAGACCCUGGUACAGGCCUGGGGGAC |
| | | | AGGGACCUGGGGAC |

The mRNA probes which were selected during cross-validation are given in Table 19. The probe sequences were obtained from Bioconductor package hu35ksubaprobe (see The Bioconductor Project, www.bioconductor.org (2008). hu35ksubaprobe: Probe sequence data for microarrays of type hu35ksuba. R package version 2.2.0.).

TABLE 19
Table 19: mRNA probes selected during LOO cross validation

| | | Times | |
|---|---|---|---|
| Seq-ID | Affymetrix ID | selected | Probe sequence |
| 298-313 | AA285290_at | 5 | [1] GGAAAGCGCCGAGATGACGGGCTTT |
| | | | [2] GATGACGGGCTTTCTGCTGCCGCCC |
| | | | [3] CCCAAGTAGCTTTGTGGCTTCGTGT |
| | | | [4] TAGCTTTGTGGCTTCGTGTCCAACC |
| | | | [5] TGTGGCTTCGTGTCCAACCCTCTTG |
| | | | [6] CGCCTGTGTGCCTGGAGCCAGTCCC |
| | | | [7] GCTCGCGTTTCCTCCTGTAGTGCTC |
| | | | [8] GTTTCCTCCTGTAGTGCTCACAGGT |
| | | | [9] AGTGCTCACAGGTCCCAGCACCGAT |
| | | | [10] TCCCAGCACCGATGGCATTCCCTTT |
| | | | [11] TCCCTTTGCCCTGAGTCTGCAGCGG |
| | | | [12] TGCCCTGAGTCTGCAGCGGGTCCCT |
| | | | [13] TCAGGTAGCCTCTCTTCCCCTTGGG |
| | | | [14] ACCCGCGGTAACCAGCGTGAGCTCG |
| | | | [15] GCCCGCCAGAAGAATATGAAAAAGC |
| | | | [16] GACTCGGTTAAGGGAAAGCGCCGAG |
| 314-328 | AA464334_s_at | 4 | [1] TTATGAATGTCCAAATCTGTGTTTC |
| | | | [2] ATGAATGTCCAAATCTGTGTTTCCC |
| | | | [3] GAATGTCCAAATCTGTGTTTCCCCC |
| | | | [4] ATGTCCAAATCTGTGTTTCCCCCTG |
| | | | [5] CTCCCAGACTGTGTGGCCAGTTGAA |
| | | | [6] AGACTGTGTGGCCAGTTGAAAGTGT |
| | | | [7] ACTGTGTGGCCAGTTGAAAGTGTCT |
| | | | [8] TGGCCAGTTGAAAGTGTCTGGTTTG |
| | | | [9] TTGAAAGTGTCTGGTTTGTGTTCAT |
| | | | [10] AGTGTCTGGTTTGTGTTCATCTCTC |
| | | | [11] TGTCTGGTTTGTGTTCATCTCTCCC |
| | | | [12] GTGTTCATCTCTCCCTCATTTCTGG |
| | | | [13] TGCATCCACGCCTCTTTTGGACATT |
| | | | [14] CATCCACGCCTCTTTTGGACATTAA |
| | | | [15] TCCACGCCTCTTTTGGACATTAAAG |
| 329-343 | AA397610_at | 3 | [1] GGTGGCCTTCTTGCAGGTCCCCGTA |
| | | | [2] TGGCCTTCTTGCAGGTCCCCGTAGC |
| | | | [3] GGCCTTCTTGCAGGTCCCCGTAGCA |
| | | | [4] GCCTTCTTGCAGGTCCCCGTAGCAC |
| | | | [5] TCTTGCAGGTCCCCGTAGCACCCTG |
| | | | [6] TGCAGGTCCCCGTAGCACCCTGAGC |
| | | | [7] AGGTCCCCGTAGCACCCTGAGCCTG |
| | | | [8] GGTCCCCGTAGCACCCTGAGCCTGT |
| | | | [9] CCGTAGCACCCTGAGCCTGTACCTT |
| | | | [10] TAGCACCCTGAGCCTGTACCTTGGG |
| | | | [11] CACCCTGAGCCTGTACCTTGGGTGG |
| | | | [12] ACCCTGAGCCTGTACCTTGGGTGGC |
| | | | [13] CCCTGAGCCTGTACCTTGGGTGGCA |
| | | | [14] GAGCCTGTACCTTGGGTGGCACTTG |
| | | | [15] GCCTGTACCTTGGGTGGCACTTGTT |
| 344-359 | RC_AA292427_s_at | 3 | [1] TGCTGCCTCTGGGGACATGCGGAGT |
| | | | [2] GGGGAAGCCTTCCTCTCAATTTGTT |
| | | | [3] GGGAAGCCTTCCTCTCAATTTGTTG |
| | | | [4] GGAAGCCTTCCTCTCAATTTGTTGT |
| | | | [5] GAAGCCTTCCTCTCAATTTGTTGTC |
| | | | [6] AAGCCTTCCTCTCAATTTGTTGTCA |
| | | | [7] AGCCTTCCTCTCAATTTGTTGTCAG |
| | | | [8] CCTTCCTCTCAATTTGTTGTCAGTG |
| | | | [9] CTTCCTCTCAATTTGTTGTCAGTGA |
| | | | [10] TTCCTCTCAATTTGTTGTCAGTGAA |
| | | | [11] TCCTCTCAATTTGTTGTCAGTGAAA |
| | | | [12] CCTCTCAATTTGTTGTCAGTGAAAT |
| | | | [13] CTCTCAATTTGTTGTCAGTGAAATT |
| | | | [14] AATTCCAATAAATGGGATTTGCTCT |
| | | | [15] TGAGGGTGCACGTCTTCCCTCCTGT |
| | | | [16] TGGAGTGCTGCCTCTGGGGACATGC |
| 360-374 | RC_AA465694_r_at | 3 | [1] GGTTAATCCGCAAGCCCCAGCCCCG |
| | | | [2] TTAATCCGCAAGCCCCAGCCCCGAG |
| | | | [3] GGCGTCCCCCAGAGCCTGAGAAAGC |
| | | | [4] CCCCAGAGCCTGAGAAAGCGCCTCC |
| | | | [5] CCAGAGCCTGAGAAAGCGCCTCCCG |
| | | | [6] GAGCCTGAGAAAGCGCCTCCCGCTG |
| | | | [7] GCCTGAGAAAGCGCCTCCCGCTGCC |
| | | | [8] CTGAGAAAGCGCCTCCCGCTGCCCC |
| | | | [9] TGCCCCGACGCGGCCCTCGGCCCTG |
| | | | [10] CTCGGCCCTGGAGCTGAAGGTGGAG |
| | | | [11] CGGCCCTGGAGCTGAAGGTGGAGGA |
| | | | [12] GCCCTGGAGCTGAAGGTGGAGGAGC |
| | | | [13] CCTGGAGCTGAAGGTGGAGGAGCTG |
| | | | [14] GCTGAAGGTGGAGGAGCTGGAGGAG |
| | | | [15] AAGGTGGAGGAGCTGGAGGAGAAGG |
| 375-390 | AA422123_f_at | 2 | [1] GACTGCTTGAAACCAGGAGTTTGAG |
| | | | [2] GCTTGAAACCAGGAGTTTGAGACCA |
| | | | [3] AACCAGGAGTTTGAGACCAGCCTGA |
| | | | [4] TTGAGACCAGCCTGAGCAACAAAGC |
| | | | [5] AGACCAGCCTGAGCAACAAAGCAAG |
| | | | [6] GAGCAACAAAGCAAGACCCCATCTC |
| | | | [7] CAACAAAGCAAGACCCCATCTCTAT |
| | | | [8] AAGCAAGACCCCATCTCTATAAAAA |
| | | | [9] AAGACAGGGTCTTGCTCATGTTGTA |
| | | | [10] ATTAGTTGGGCATGGTGGCACATGC |
| | | | [11] AGTTGGGCATGGTGGCACATGCCTG |
| | | | [12] ATCATCTGAGCCTCAGGAGGTTGAG |
| | | | [13] ATCTGAGCCTCAGGAGGTTGAGGCT |
| | | | [14] TGAGGCTGCAGTGAGCTGTGACTGC |
| | | | [15] CTTGCTCATGTTGTACATTCATCAT |
| | | | [16] AAGAGGCTGGGTGCAGTGGCTCACA |
| 391-410 | AFFX- | 2 | [1] TCATTTCCTGGTATGACAACGAATT |
| | HUMGAPDH/M33197_3_at | | [2] ACAACGAATTTGGCTACAGCAACAG |
| | | | [3] GGGTGGTGGACCTCATGGCCCACAT |
| | | | [4] TCATGGCCCACATGGCCTCCAAGGA |
| | | | [5] ACATGGCCTCCAAGGAGTAAGACCC |
| | | | [6] AGGAGTAAGACCCCTGGACCACCAG |
| | | | [7] GCCCCAGCAAGAGCACAAGAGGAAG |
| | | | [8] GAGAGAGACCCTCACTGCTGGGGAG |
| | | | [9] CCTCACTGCTGGGGAGTCCCTGCCA |
| | | | [10] CCTCCTCACAGTTGCCATGTAGACC |
| | | | [11] AGTTGCCATGTAGACCCCTTGAAGA |
| | | | [12] CATGTAGACCCCTTGAAGAGGGGAG |
| | | | [13] TAGGGAGCCGCACCTTGTCATGTAC |
| | | | [14] GCCGCACCTTGTCATGTACCATCAA |
| | | | [15] TGTCATGTACCATCAATAAAGTACC |
| | | | [16] CCTCTGACTTCAACAGCGACACCCA |
| | | | [17] GGGCTGGCATTGCCCTCAACGACCA |
| | | | [18] CCCTCAACGACCACTTTGTCAAGCT |
| | | | [19] ACCACTTTGTCAAGCTCATTTCCTG |
| | | | [20] TTGTCAAGCTCATTTCCTGGTATGA |
| 411-426 | RC_AA130645_s_at | 2 | [1] GAATTCTGGTACCGTCAGCATCCAC |
| | | | [2] GAGAGAGACCTCATCTTTCATGCTT |
| | | | [3] TGACTCTCCTGGGGGCACCTCCTAT |
| | | | [4] ACTCTCCTGGGGGCACCTCCTATGA |
| | | | [5] TCCTGGGGGCACCTCCTATGAGAGA |
| | | | [6] CCTGGGGGCACCTCCTATGAGAGAT |
| | | | [7] CTGGGGGCACCTCCTATGAGAGATA |
| | | | [8] TGGGGGCACCTCCTATGAGAGATAC |
| | | | [9] GGGGGCACCTCCTATGAGAGATACG |
| | | | [10] GGGGCACCTCCTATGAGAGATACGA |
| | | | [11] GGGCACCTCCTATGAGAGATACGAT |
| | | | [12] GGCACCTCCTATGAGAGATACGATT |
| | | | [13] GCACCTCCTATGAGAGATACGATTG |
| | | | [14] CACCTCCTATGAGAGATACGATTGC |
| | | | [15] ACCTCCTATGAGAGATACGATTGCT |
| | | | [16] CCTCCTATGAGAGATACGATTGCTA |
| 427-442 | RC_AA236365_s_at | 2 | [1] CTCCTATTCCGGACTCAGACCTCTG |
| | | | [2] TCCTATTCCGGACTCAGACCTCTGA |
| | | | [3] CCTATTCCGGACTCAGACCTCTGAC |
| | | | [4] CTATTCCGGACTCAGACCTCTGACC |
| | | | [5] ATTCCGGACTCAGACCTCTGACCCT |
| | | | [6] TTCCGGACTCAGACCTCTGACCCTG |
| | | | [7] CGGACTCAGACCTCTGACCCTGCAA |
| | | | [8] GGACTCAGACCTCTGACCCTGCAAT |
| | | | [9] ACTCAGACCTCTGACCCTGCAATGC |
| | | | [10] CAGACCTCTGACCCTGCAATGCTGC |
| | | | [11] ACCTCTGACCCTGCAATGCTGCCTA |
| | | | [12] TCTGACCCTGCAATGCTGCCTACCA |
| | | | [13] CTGACCCTGCAATGCTGCCTACCAT |
| | | | [14] TGACCCTGCAATGCTGCCTACCATG |
| | | | [15] ACCCTGCAATGCTGCCTACCATGAT |
| | | | [16] CCTGCAATGCTGCCTACCATGATTG |
| 443-458 | RC_AA304344_f_at | 2 | [1] AGGCACGTACCACCATGCCCAGATA |
| | | | [2] TTTTTTGAGACAAAGTCCTCACTCT |
| | | | [3] GGGGTTTCACCATGTTGGCTAGGAT |
| | | | [4] CCATGTTGGCTAGGATGGTCTCCAT |
| | | | [5] GTTGGCTAGGATGGTCTCCATCGCC |
| | | | [6] CTAGGATGGTCTCCATCGCCTGACC |
| | | | [7] TGAGACAAAGTCCTCACTCTGTCAC |
| | | | [8] CTTGGCCTCCCAAAGTGCTGGGATT |
| | | | [9] CCTCCCAAAGTGCTGGGATTACAGG |
| | | | [10] GGATTACAGGCATGAGCCACCACAG |
| | | | [11] CAAAGTCCTCACTCTGTCACCAAGT |
| | | | [12] GCATGAGCCACCACAGCTGGCCGTA |
| | | | [13] GAGCCACCACAGCTGGCCGTAAATA |
| | | | [14] GTGCAGTGGCAGCAATCTCAGCTCA |
| | | | [15] GTGGCAGCAATCTCAGCTCACTGCA |
| | | | [16] AGCAATCTCAGCTCACTGCAAACCT |
| 459-473 | T89571_f_at | 2 | [1] CACCGCGCCTGGCCCTAAATAGATT |
| | | | [2] GGGATTCATCATGTTGACCAGGCTG |
| | | | [3] TTCATCATGTTGACCAGGCTGGCCT |
| | | | [4] TGTTTGTCTTTCTGATAGGTTGAAA |
| | | | [5] TGTCTTTCTGATAGGTTGAAAATTG |
| | | | [6] GTTGACCAGGCTGGCCTCAAACTCC |
| | | | [7] ACCAGGCTGGCCTCAAACTCCTGAC |
| | | | [8] AGGCTGGCCTCAAACTCCTGACTTC |
| | | | [9] TGGCCTCAAACTCCTGACTTCAAGC |
| | | | [10] CTCAAACTCCTGACTTCAAGCGATC |
| | | | [11] AAACTCCTGACTTCAAGCGATCTCC |
| | | | [12] TTGGCCTCCCAAAGTGCTGGGATTG |
| | | | [13] CCTCCCAAAGTGCTGGGATTGCAGG |
| | | | [14] GCTGGGATTGCAGGTGTGAGCCACC |
| | | | [15] ATTGCAGGTGTGAGCCACCGCGCCT |
| 474-493 | AFFX- | 1 | [1] TCTTGACAAAACCTAACTTGCGCAG |
| | HSAC07/X00351_3_at | | [2] ATGAGATTGGCATGGCTTTATTTGT |
| | | | [3] GCAGTCGGTTGGAGCGAGCATCCCC |
| | | | [4] CCAAAGTTCACAATGTGGCCGAGGA |
| | | | [5] AAGTTCACAATGTGGCCGAGGACTT |
| | | | [6] ATGTGGCCGAGGACTTTGATTGCAC |
| | | | [7] CCGAGGACTTTGATTGCACATTGTT |
| | | | [8] TTTAATAGTCATTCCAAATATGAGA |
| | | | [9] AGTCATTCCAAATATGAGATGCATT |
| | | | [10] TGTTACAGGAAGTCCCTTGCCATCC |
| | | | [11] TACAGGAAGTCCCTTGCCATCCTAA |
| | | | [12] TCCCTTGCCATCCTAAAAGCCACCC |
| | | | [13] CTTCTCTCTAAGGAGAATGGCCCAG |
| | | | [14] GAGGTGATAGCATTGCTTTCGTGTA |
| | | | [15] TATTTTGAATGATGAGCCTTCGTGC |
| | | | [16] TTTGAATGATGAGCCTTCGTGCCCC |
| | | | [17] GTATGAAGGCTTTTGGTCTCCCTGG |
| | | | [18] GGTGGAGGCAGCCAGGGCTTACCTG |
| | | | [19] CAGGGCTTACCTGTACACTGACTTG |
| | | | [20] TTACCTGTACACTGACTTGAGACCA |
| 494-562 | hum_alu_at | 1 | [1] GCCTGGCCAACATGGTGAAACCCCG |
| | | | [2] GCGCGCGCCTGTAATCCCAGCTACT |
| | | | [3] GCGCGCCTGTAATCCCAGCTACTCG |
| | | | [4] CGCGCCTGTAATCCCAGCTACTCGG |
| | | | [5] GCGCCTGTAATCCCAGCTACTCGGG |
| | | | [6] CGCCTGTAATCCCAGCTACTCGGGA |
| | | | [7] GCCTGTAATCCCAGCTACTCGGGAG |
| | | | [8] CCTGTAATCCCAGCTACTCGGGAGG |
| | | | [9] CTGTAATCCCAGCTACTCGGGAGGC |
| | | | [10] TGTAATCCCAGCTACTCGGGAGGCT |
| | | | [11] GTAATCCCAGCTACTCGGGAGGCTG |
| | | | [12] TAATCCCAGCTACTCGGGAGGCTGA |
| | | | [13] AATCCCAGCTACTCGGGAGGCTGAG |
| | | | [14] ATCCCAGCTACTCGGGAGGCTGAGG |
| | | | [15] TCCCAGCTACTCGGGAGGCTGAGGC |
| | | | [16] CCCAGCTACTCGGGAGGCTGAGGCA |
| | | | [17] CCAGCTACTCGGGAGGCTGAGGCAG |
| | | | [18] TGGTGGCTCACGCCTGTAATCCCAG |
| | | | [19] GAGCCGAGATCGCGCCACTGCACTC |
| | | | [20] GTGGCTCACGCCTGTAATCCCAGCA |
| | | | [21] CACTGCACTCCAGCCTGGGCGACAG |
| | | | [22] ACTGCACTCCAGCCTGGGCGACAGA |
| | | | [23] CTGCACTCCAGCCTGGGCGACAGAG |
| | | | [24] TGCACTCCAGCCTGGGCGACAGAGC |
| | | | [25] GCACTCCAGCCTGGGCGACAGAGCG |
| | | | [26] CACTCCAGCCTGGGCGACAGAGCGA |
| | | | [27] TGGCTCACGCCTGTAATCCCAGCAC |
| | | | [28] ACTCCAGCCTGGGCGACAGAGCGAG |
| | | | [29] CTCCAGCCTGGGCGACAGAGCGAGA |
| | | | [30] TCCAGCCTGGGCGACAGAGCGAGAC |
| | | | [31] CCAGCCTGGGCGACAGAGCGAGACT |
| | | | [32] CAGCCTGGGCGACAGAGCGAGACTC |
| | | | [33] AGCCTGGGCGACAGAGCGAGACTCC |
| | | | [34] GGCTCACGCCTGTAATCCCAGCACT |
| | | | [35] GCTCACGCCTGTAATCCCAGCACTT |
| | | | [36] CTCACGCCTGTAATCCCAGCACTTT |
| | | | [37] TCACGCCTGTAATCCCAGCACTTTG |
| | | | [38] CACGCCTGTAATCCCAGCACTTTGG |
| | | | [39] ACGCCTGTAATCCCAGCACTTTGGG |
| | | | [40] CGCCTGTAATCCCAGCACTTTGGGA |
| | | | [41] GCCTGTAATCCCAGCACTTTGGGAG |
| | | | [42] CCTGTAATCCCAGCACTTTGGGAGG |
| | | | [43] CTGTAATCCCAGCACTTTGGGAGGC |
| | | | [44] TGTAATCCCAGCACTTTGGGAGGCC |
| | | | [45] GTAATCCCAGCACTTTGGGAGGCCG |
| | | | [46] TAATCCCAGCACTTTGGGAGGCCGA |
| | | | [47] AATCCCAGCACTTTGGGAGGCCGAG |
| | | | [48] ATCCCAGCACTTTGGGAGGCCGAGG |
| | | | [49] TCCCAGCACTTTGGGAGGCCGAGGT |
| | | | [50] CCCAGCACTTTGGGAGGCCGAGGTG |
| | | | [51] GTGGATCACCTGAGGTCAGGAGTTC |
| | | | [52] GGATCACCTGAGGTCAGGAGTTCAA |
| | | | [53] GATCACCTGAGGTCAGGAGTTCAAG |
| | | | [54] ATCACCTGAGGTCAGGAGTTCAAGA |
| | | | [55] TCACCTGAGGTCAGGAGTTCAAGAC |
| | | | [56] AGGAGTTCAAGACCAGCCTGGCCAA |
| | | | [57] GGAGTTCAAGACCAGCCTGGCCAAC |
| | | | [58] GAGTTCAAGACCAGCCTGGCCAACA |
| | | | [59] AGTTCAAGACCAGCCTGGCCAACAT |
| | | | [60] GTTCAAGACCAGCCTGGCCAACATG |
| | | | [61] TTCAAGACCAGCCTGGCCAACATGG |
| | | | [62] TCAAGACCAGCCTGGCCAACATGGT |
| | | | [63] CAAGACCAGCCTGGCCAACATGGTG |
| | | | [64] AAGACCAGCCTGGCCAACATGGTGA |
| | | | [65] AGACCAGCCTGGCCAACATGGTGAA |
| | | | [66] GACCAGCCTGGCCAACATGGTGAAA |
| | | | [67] ACCAGCCTGGCCAACATGGTGAAAC |
| | | | [68] CCAGCCTGGCCAACATGGTGAAACC |
| | | | [69] CAGCCTGGCCAACATGGTGAAACCC |
| 563-578 | R69648_at | 1 | [1] TAGAATTCTGTGCAGATGTCCTGAC |
| | | | [2] AATTCTGTGCAGATGTCCTGACTTG |
| | | | [3] TGACTTGGCAATTTTGTGTCCCTGC |
| | | | [4] GGCAATTTTGTGTCCCTGCCTCACT |
| | | | [5] GTCCTAGTGTTGTTCTGCCTCCTGT |
| | | | [6] TTGTTCTGCCTCCTGTCCTCTCTTG |
| | | | [7] CTGTCCTCTCTTGCTCTCTTGTCAG |
| | | | [8] GCTCTCTTGTCAGTCTCTGGCTTCC |
| | | | [9] GTCTCTGGCTTCCTCGGCCCCATTT |
| | | | [10] GGCCCCATTTCACTTCACTGAGTCC |
| | | | [11] CCCATTTCACTTCACTGAGTCCTGA |
| | | | [12] TCACTTCACTGAGTCCTGACACCCA |
| | | | [13] AAGGGTCTGTTCTGCTCAGCTCCAT |
| | | | [14] TGCTCAGCTCCATGTCCCCCATTTT |
| | | | [15] TTTACAGCATCCTGCACTCCAGCCT |
| | | | [16] TCCTCCACAATAAAACTGGGGACTG |
| 579-593 | RC_AA232686_s_at | 1 | [1] GCTGAGGCTCCCTTGCCTGACTGTG |
| | | | [2] GAGGCTCCCTTGCCTGACTGTGACT |
| | | | [3] GGCTCCCTTGCCTGACTGTGACTTG |
| | | | [4] GCTCCCTTGCCTGACTGTGACTTGT |
| | | | [5] CTCCCTTGCCTGACTGTGACTTGTG |
| | | | [6] TCCCTTGCCTGACTGTGACTTGTGC |
| | | | [7] CCCTTGCCTGACTGTGACTTGTGCC |
| | | | [8] CCTTGCCTGACTGTGACTTGTGCCT |
| | | | [9] CTTGCCTGACTGTGACTTGTGCCTC |
| | | | [10] CTGACTGTGACTTGTGCCTCTCTCC |
| | | | [11] TGACTGTGACTTGTGCCTCTCTCCT |
| | | | [12] GACTGTGACTTGTGCCTCTCTCCTG |
| | | | [13] CTGTGACTTGTGCCTCTCTCCTGCC |
| | | | [14] GGTGGGCAGGTGACCCAAGGAACCT |
| | | | [15] CAGGTGACCCAAGGAACCTTTCTGG |
| 594-609 | RC_AA417588_at | 1 | [1] TGAAGGTACTGAACGCCACCTCACT |
| | | | [2] AGGTACTGAACGCCACCTCACTGTA |
| | | | [3] GTACTGAACGCCACCTCACTGTAAG |
| | | | [4] TGAACGCCACCTCACTGTAAGACGG |
| | | | [5] AACGCCACCTCACTGTAAGACGGTA |
| | | | [6] ACGCCACCTCACTGTAAGACGGTAG |
| | | | [7] GCCACCTCACTGTAAGACGGTAGAT |
| | | | [8] CCACCTCACTGTAAGACGGTAGATT |
| | | | [9] ACCTCACTGTAAGACGGTAGATTTT |
| | | | [10] CCTCACTGTAAGACGGTAGATTTTG |
| | | | [11] TCACTGTAAGACGGTAGATTTTGTA |
| | | | [12] GACAGGGCTGCCTTCTGGGTGATGA |
| | | | [13] ACAGGGCTGCCTTCTGGGTGATGAG |
| | | | [14] AGGGCTGCCTTCTGGGTGATGAGAA |
| | | | [15] AATCAGATGGGATGGCTGCACGGCG |
| | | | [16] CTGCACGGCGTGGTGAAGGTACTGA |
| 610-624 | RC_AA459310_r_at | 1 | [1] CTGCAGTTCATGTCCCCCGCCAGGC |
| | | | [2] CCCCGCCAGGCCTCGAGGCTCAGGG |
| | | | [3] CGCCAGGCCTCGAGGCTCAGGGTGG |
| | | | [4] GCCTCGAGGCTCAGGGTGGGAGAGG |
| | | | [5] GAGGCTCAGGGTGGGAGAGGGCCCC |
| | | | [6] GCTCAGGGTGGGAGAGGGCCCCGGG |
| | | | [7] CCCCGGGCTGCCCTGTCACTCCTCT |
| | | | [8] CGGGCTGCCCTGTCACTCCTCTAAC |
| | | | [9] GCTGCCCTGTCACTCCTCTAACACT |
| | | | [10] CCTGTCACTCCTCTAACACTTCCCT |
| | | | [11] TCACTCCTCTAACACTTCCCTCCCG |
| | | | [12] CTCCTCTAACACTTCCCTCCCGTGT |
| | | | [13] CCCCAACATGCCCTGTAATAAAATT |
| | | | [14] CAACATGCCCTGTAATAAAATTAGA |
| | | | [15] CATGCCCTGTAATAAAATTAGAGAA |
| 625-639 | RC_AA496904_at | 1 | [1] TAGAATGACCCTTGGGAACAGTGAA |
| | | | [2] GACCCTTGGGAACAGTGAACGTAGA |
| | | | [3] TTTAGCAGAGTTTGTGACCAAAGTC |
| | | | [4] GCTCTGGCTGCCTTCTGCATTTATT |
| | | | [5] GCTGCCTTCTGCATTTATTTGCCTT |
| | | | [6] GCCTTGGCCTGTTGTCTTCCCCTAT |
| | | | [7] GCCTGTTGTCTTCCCCTATTTTCTG |
| | | | [8] TGTCTTCCCCTATTTTCTGTCCCAG |
| | | | [9] CTATTTTCTGTCCCAGCTCATCCGT |
| | | | [10] TTTTCTGTCCCAGCTCATCCGTGTC |
| | | | [11] TCTGTCCCAGCTCATCCGTGTCTCT |
| | | | [12] GTCCCAGCTCATCCGTGTCTCTGAA |
| | | | [13] CCAGCTCATCCGTGTCTCTGAAGAA |
| | | | [14] GCTCATCCGTGTCTCTGAAGAACAA |
| | | | [15] CCGTGTCTCTGAAGAACAAATATGC |
| 640-654 | RC_D59847_at | 1 | [1] TTGCCACCCTGAGCACTGCCCGGAT |
| | | | [2] GGATCCCGTGCACCCTGGGACCCAG |
| | | | [3] TCCCGTGCACCCTGGGACCCAGAAG |
| | | | [4] CGTGCACCCTGGGACCCAGAAGTGC |
| | | | [5] CCGCCAGCACGTCCAGAGCAACTTA |
| | | | [6] GCCAGCACGTCCAGAGCAACTTACC |
| | | | [7] AGCACGTCCAGAGCAACTTACCCCG |
| | | | [8] GCACGTCCAGAGCAACTTACCCCGG |
| | | | [9] CCGTGCCGCCGACCACGATGTGGGC |
| | | | [10] CGTGCCGCCGACCACGATGTGGGCT |
| | | | [11] TGCCGCCGACCACGATGTGGGCTCT |
| | | | [12] CGCCGACCACGATGTGGGCTCTGAG |
| | | | [13] GACCACGATGTGGGCTCTGAGCTGC |
| | | | [14] CACGATGTGGGCTCTGAGCTGCCCC |
| | | | [15] TGTGAAACGCCTAGAGACCCCGGCG |
| 655-669 | RC_D60607_at | 1 | [1] TCACAGCCCCGTTCAGCTGGTGGCT |
| | | | [2] CCCCGTTCAGCTGGTGGCTTTTAGA |
| | | | [3] TTTTAGAGGCTTCCAGAGTGTGCTT |
| | | | [4] CCAGAGTGTGCTTGGCCCCTTTACC |
| | | | [5] TGGCCCCTTTACCTCTATGCCATTG |
| | | | [6] CTCTATGCCATTGGGCCCAGGGGGA |
| | | | [7] CCTTTCTGTGTCTTGCTTGCCCCGT |
| | | | [8] TGTGTCTTGCTTGCCCCGTGTCTCC |
| | | | [9] TTGCTTGCCCCGTGTCTCCCAGTGA |
| | | | [10] GCCCCGTGTCTCCCAGTGAGTGGCC |
| | | | [11] TGTCTCCCAGTGAGTGGCCGCCCTG |
| | | | [12] CGGACAAGTCGCAGCCTCAGGGGGA |
| | | | [13] AGTCGCAGCCTCAGGGGGACCTCCC |
| | | | [14] CTGGCACTGCATCTTTCTGGGCCTG |
| | | | [15] CTTTCTGGGCCTGGCTCTGCTGCCT |
| 670-684 | T30851_i_at | 1 | [1] CAGAGTTATAAGCCCCAAACAGGTC |
| | | | [2] AGAGTTATAAGCCCCAAACAGGTCA |
| | | | [3] GAGTTATAAGCCCCAAACAGGTCAT |
| | | | [4] AGTTATAAGCCCCAAACAGGTCATG |
| | | | [5] GTTATAAGCCCCAAACAGGTCATGC |
| | | | [6] TTATAAGCCCCAAACAGGTCATGCT |
| | | | [7] TATAAGCCCCAAACAGGTCATGCTC |
| | | | [8] ATAAGCCCCAAACAGGTCATGCTCC |
| | | | [9] TAAGCCCCAAACAGGTCATGCTCCA |
| | | | [10] AAGCCCCAAACAGGTCATGCTCCAA |
| | | | [11] AGCCCCAAACAGGTCATGCTCCAAT |
| | | | [12] GCCCCAAACAGGTCATGCTCCAATA |
| | | | [13] CCCCAAACAGGTCATGCTCCAATAA |
| | | | [14] CCCAAACAGGTCATGCTCCAATAAA |
| | | | [15] CCAAACAGGTCATGCTCCAATAAAA |
| 685-700 | T80746_s_at | 1 | [1] CTTGCAACCTCCGGGACCATCTTCT |
| | | | [2] GCAACCTCCGGGACCATCTTCTCGG |
| | | | [3] GCTTCTGGGACCTGCCAGCACCGTT |
| | | | [4] GGGACCTGCCAGCACCGTTTTTGTG |
| | | | [5] TGCCAGCACCGTTTTTGTGGTTAGC |
| | | | [6] CAGCACCGTTTTTGTGGTTAGCTCC |
| | | | [7] TTGCCAACCAACCATGAGCTCCCAG |
| | | | [8] GCCAACCAACCATGAGCTCCCAGAT |
| | | | [9] AACCAACCATGAGCTCCCAGATTCG |
| | | | [10] CCATGAGCTCCCAGATTCGTCAGAA |
| | | | [11] TGAGCTCCCAGATTCGTCAGAATTA |
| | | | [12] GCTCCCAGATTCGTCAGAATTATTC |
| | | | [13] CCCAGATTCGTCAGAATTATTCCAC |
| | | | [14] GATTCGTCAGAATTATTCCACCGAC |
| | | | [15] TCGTCAGAATTATTCCACCGACGTG |
| | | | [16] TCAGAATTATTCCACCGACGTGGAG |
| 701-716 | X01677_s_at | 1 | [1] ACTGGCATGGCCTTCCGTGTCCCCA |
| | | | [2] CCACTGCCAACGTGTCAGTGGTGGA |
| | | | [3] ACTGCCAACGTGTCAGTGGTGGACC |
| | | | [4] TGCCAACGTGTCAGTGGTGGACCTG |
| | | | [5] CCAACGTGTCAGTGGTGGACCTGAC |
| | | | [6] CGTGTCAGTGGTGGACCTGACCTGC |
| | | | [7] GTCAGTGGTGGACCTGACCTGCCGT |
| | | | [8] CAGTGGTGGACCTGACCTGCCGTCT |
| | | | [9] GTGGTGGACCTGACCTGCCGTCTAG |
| | | | [10] GGTGGACCTGACCTGCCGTCTAGAA |
| | | | [11] GACCTGACCTGCCGTCTAGAAAAAC |
| | | | [12] CTGACCTGCCGTCTAGAAAAACCTG |
| | | | [13] GACCTGCCGTCTAGAAAAACCTGCC |
| | | | [14] TGCCGTCTAGAAAAACCTGCCAAAT |
| | | | [15] CCGTCTAGAAAAACCTGCCAAATAT |
| | | | [16] GTCTAGAAAAACCTGCCAAATATGA |

The annotations of the selected mRNA probes are given in Table 20. The annotations were obtained from Bioconductor package hu35ksuba.db (Marc Carlson, Seth Falcon, Herve Pages and Nianhua Li (2008). hu35ksuba.db: Affymetrix Human Genome HU35K Set annotation data (chip hu35ksuba). R package version 2.2.3.) in combination with the information available via PubMed [http://www.ncbi.nlm.nih.gov/pubmed/].

TABLE 20
Table 20: Annotation of mRNA probes selected during LOO cross
validation (1^{st} column is SEQ-ID-No)

| | | | | |
|---|---|---|---|---|
| 268 | X01677_s_at | X01677 | NM_002046.3 | Hs.544577 |

### Example 2.3

### mRNA and microRNA, Prostate Cancer

We use the prostate cancer data of Ramaswamy et al. (2001) [Ramaswamy S, Tamayo P, Rifkin R, Mukherjee S, Yeang C H, Angelo M, Ladd C, Reich M, Latulippe E, Mesirov J P, Poggio T, Gerald W, Loda M, Lander E S, Golub T R. Multiclass cancer diagnosis using tumor gene expression signatures. Proc Natl Acad Sci USA. 2001; 98(26):15149-54] and Lu et al. (2005) [Lu J, Getz G, Miska E A, Alvarez-Saavedra E, Lamb J, Peck D, Sweet-Cordero A, Ebert B L, Mak R H, Ferrando A A, Downing J R, Jacks T, Horvitz H R, Golub T R. MicroRNA expression profiles classify human cancers. Nature. 2005; 435(7043):834-8] to develop a multilevel classifier using mRNA and microRNA data. The data are available from the home page of the Braoad Institute [see http://www.broad.mit.edu/publications/broad900 and http://www.broad.mit.edu/publications/broad993s]. Overall the mRNA and microRNA data of six normal tissues and six tumor tissues are available. The hybridisations were done with a bead-based array containing microRNA probes as well as with the Affymetrix HU6800 and HU35KsubA array for measuring the mRNA. We used only the mRNA data of the HU6800 arrays.

Analysis:

For developing and validating a classifier based on these data we used linear discriminant analysis in combination with leave-one-out (LOO) cross-validation where each analysis step—including low level analysis—was repeated in each cross-validation step. This is one possibility. Of course, we could also have used a split-sample, a bootstrap or a different k-fold (k not equal to 1) cross-validation approach. Moreover, we could have used a different class of functions for classification e.g. logistic regression, (diagonal) linear or quadratic discriminant analysis (LDA, QDA, DLDA, DQDA), shrunken centroids regularized discriminant analysis (RDA), random forests (RF), neural networks (NN), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more.

The low level analysis consisted of the variance stabilizing transformation of Huber et al (2002) (often called normalization) in case of the microRNA as well as of the mRNA data. Again there is a large number of alternative methods which could be used Several examples are given in Cope et al. (2004) or Irizarry et al. (2006) In each cross validation step we selected those two normalized microRNA probes, respectively those four normalized mRNA probes for classification which had the largest median of pairwise differences (in absolute value) beyond those microRNA probes with p value equal or smaller than 0.01 by the Mann-Whitney test. This is, we used a so called ranker for feature selection. Again there are numerous other feature selection strategies we could have used, some examples are given in Hall et al. 2003. Overall a microRNA, respectively mRNA probe may have been chosen up to twelve times due to LOO cross-validation.

Using only microRNA data we obtain the estimated errors given in Table 21

TABLE 21
Table 21: microRNA data, classification error via LOO cross validation

| classifier vs. true | prostate cancer | Normal |
|---|---|---|
| prostate cancer | 83.3% | 0.0% |
| normal | 16.7% | 100.0% |

The estimated overall accuracy using LOO cross-validation is 91.7%. Sensitivity, specificity, positive and negative predictive value are 83.3%, 100%, 100% and 85.7%, respectively. In a second step we used the mRNA data of the HU6800 array. The results can be read off from Table 22. We get an estimated overall accuracy of 75.0% again using LOO cross-validation.

Sensitivity, specificity, positive and negative predictive value are 83.3%, 66.7%, 71.4% and 80.0%, respectively.

TABLE 22
Table 22: mRNA data, classification error via LOO cross validation

| classifier vs. true | prostate cancer | Normal |
|---|---|---|
| prostate cancer | 83.3% | 33.3% |
| normal | 16.7% | 66.7% |

In the last step we combine microRNA and mRNA data and obtain the results given in Table 23. That is, the estimated overall accuracy using cross-validation is 91.7%. Sensitivity, specificity, positive and negative predictive value are 100.0%, 83.3%, 85.7% and 100.0%, respectively. Hence, this combination increases the sensitivity (correct classification of cancer samples) from 83.3% to 100.0% and negative predictive value form 85.7%, respectively 80.0% to 100.0%.

TABLE 23
Table 23: microRNA and mRNA data, classification error via
LOO cross validation

| classifier vs. true | prostate cancer | Normal |
|---|---|---|
| prostate cancer | 100.0% | 16.7% |
| normal | 0.0% | 83.3% |

The microRNA probes which were selected during cross-validation are given in Table 24.

TABLE 24
Table 24: microRNA probes selected during LOO
cross validation (1^{st} column is SEQ-ID-No)

| | | | |
|---|---|---|---|
| 735 | hsa-miR-206 | 2 | CCACACACTTCCTTACATTCCA |

The results of the Sanger sequence search according to Griffiths-Jones et al. (2008) for known human microRNAs are given in Table 25

TABLE 25
Table 25: Results of the Sanger sequence search1
for known human microRNAs for microRNA probes
selected during LOO cross validation (1^{st} column
is SEQ-ID-No)

| | | | |
|---|---|---|---|
| 738 | hsa-miR- | hsa-mir- | UGCUUCCCGAGGCCACAUGCUUCUUUAUAU |
| | 206 | 206 | CCCCAUAUGGAUUACUUUGCUAUGGAAUGU |
| | | | AAGGAAGUGUGUGGUUUCGGCAAGUG |

The mRNA probes which were selected during cross-validation are given in Table 26. The probe sequences were obtained from Bioconductor package hu6800probe [The Bioconductor Project, www.bioconductor.org (2008). hu6800probe: Probe sequence data for microarrays of type hu6800. R package version 2.2.0].

TABLE 26
Table 26: mRNA probes selected during LOO
cross validation

| | | | |
|---|---|---|---|
| 833-852 | S82297_at | 2 | [1] GCTATCCAGCATTCAGGTTTACTCA |
| | | | [2] ATCCTGAAGCTGACAGCATTCGGGC |
| | | | [3] CCTGAAGCTGACAGCATTCGGGCCG |
| | | | [4] AAGCTGACAGCATTCGGGCCGAGAT |
| | | | [5] GCTGACAGCATTCGGGCCGAGATGT |
| | | | [6] TGACAGCATTCGGGCCGAGATGTCT |
| | | | [7] CATTCGGGCCGAGATGTCTCGCTCC |
| | | | [8] GGCCGAGATGTCTCGCTCCGTGGCC |
| | | | [9] GGAGGTTTGAAGATGCCGCAGGATC |
| | | | [10] GAGATGTCTCGCTCCGTGGCCTTAG |
| | | | [11] GATGTCTCGCTCCGTGGCCTTAGCT |
| | | | [12] TGTCTCGCTCCGTGGCCTTAGCTGT |
| | | | [13] CGTGGCCTTAGCTGTGCTCGCGCTA |
| | | | [14] CTTAGCTGTGCTCGCGCTACTCTCT |
| | | | [15] TAGCTGTGCTCGCGCTACTCTCTCT |
| | | | [16] GCTGTGCTCGCGCTACTCTCTCTTT |
| | | | [17] TGTGCTCGCGCTACTCTCTCTTTCT |
| | | | [18] GCCTGGAGGCTATCCAGCATTCAGG |
| | | | [19] CTGGAGGCTATCCAGCATTCAGGTT |
| | | | [20] GGAGGCTATCCAGCATTCAGGTTTA |
| 873-892 | J02611_at | 1 | [1] TGAGAAGATCCCAACAACCTTTGAG |
| | | | [2] GATCCCAACAACCTTTGAGAATGGA |
| | | | [3] CTTTGAGAATGGACGCTGCATCCAG |
| | | | [4] ACGCTGCATCCAGGCCAACTACTCA |
| | | | [5] CATCCAGGCCAACTACTCACTAATG |
| | | | [6] TTCCTGGTTTATGCCATCGGCACCG |
| | | | [7] GTTTATGCCATCGGCACCGTACTGG |
| | | | [8] GATCCTGGCCACCGACTATGAGAAC |
| | | | [9] GGCCACCGACTATGAGAACTATGCC |
| | | | [10] TGAGAACTATGCCCTCGTGTATTCC |
| | | | [11] CCTCGTGTATTCCTGTACCTGCATC |
| | | | [12] GTATTCCTGTACCTGCATCATCCAA |
| | | | [13] CTGTACCTGCATCATCCAACTTTTT |
| | | | [14] CTGCATCATCCAACTTTTTCACGTG |
| | | | [15] TGCTTGGATCTTGGCAAGAAACCCT |
| | | | [16] CACAGACCAGGTGAACTGCCCCAAG |
| | | | [17] CCAGGTGAACTGCCCCAAGCTCTCG |
| | | | [18] AGGTTCTACAGGGAGGCTGCACCCA |
| | | | [19] ACTCCATGTTACTTCTGCTTCGCTT |
| | | | [20] CCTGTTACCTTGCTAGCTGCAAAAT |

The annotations of the selected mRNA probes are given in Table 27. The annotations were obtained from Bioconductor package hu6800.db [Marc Carlson, Seth Falcon, Herve Pages and Nianhua Li (2008). hu6800.db: Affymetrix HuGeneFL Genome Array annotation data (chip hu6800). R package version 2.2.3.] in combination with the information available via PubMed [http://www.ncbi.nlm.nih.gov/pubmed/].

TABLE 27
Table 27: Annotation of mRNA probes selected during LOO cross
validation (1^{st} column is SEQ-ID-No)

| | | | | |
|---|---|---|---|---|
| 900 | J02611_at | J02611 | NM_001647.2 | Hs.522555 |

### Example 3

### Metabolites and mRNA: Ischemia/Hypoxia

### Ischemia and Hypoxia

Early diagnosis will buy critical time for timely intervention and selection of the appropriate therapy and thus to prevent fatal permanent brain damage

As for infants, in industrial countries the percentage of preterm subjects has increased during the last decades and now risen up to 12% of all live births [Martin J A, Hamilton B E Sutton P D et al. Births: final data for 2004. Natl Vital Stat Rep. 2006; 55:1-101; Martin J A, Hamilton B E, Sutton P D et al. Births: final data for 2005. Natl Vital Stat Rep. 2007; 56:1-103].

However, developmental brain injury and the subsequent neurological sequelae are still a major personal burden for affected individuals and their families and constitutes a considerable socioeconomic problem.

Early detection of a status of ischemia/hypoxia or stroke in man or of perinatal brain lesions in adult patients and preterm infants will enable and the application of successful therapeutic regimens and allow to control the consequences of these measures.

We use the ischemia data obtained from a rat hypoxia model to develop a multi-level classifier using metabolite data from brain samples and qPCR data from plasma.

Animal Model

A model of HI brain injury based on Rice-Vanucci's procedure was performed at postnatal day 7 (P7) [Rice J E, III, Vannucci R C, Brierley J B. The influence of immaturity on hypoxic-ischemic brain damage in the rat. Ann Neurol. 1981; 9:131-141] Sprague-Dawley rat pups (from Charles River, Wilmington, Mass., U.S.A.) of either sex were randomly assigned a) the experimental groups and b) the time. For operation animals were anesthetized with inhaled isoflurane 3% in 02, the right carotid artery was accessed through a midline incision and surgical ligation was performed with a double suture and a permanent incision. The procedure was performed at room temperature (23-25° C.) After closure of the neck wound, pups were returned to their dams for 2 h. The entire surgical procedure lasted no longer than 10 min. The pups were then exposed to hypoxia at 8% oxygen for 100 minutes. Adequate measures were taken to minimize pain and discomfort, complying with the European Community guidelines for the use of experimental animals. The study protocol was approved by the Austrian committee for animal experiments.

Sham-operated animals underwent anesthesia, neck incision and vessel manipulation without ligation or hypoxia. Control animals were kept without any damage. Animals were euthanized i) immediately after hypoxia (P7), ii) after 24 hrs (P8), iii) after 5 days (P12), brains were collected, rinsed with PBS and immediately frozen in liquid nitrogen and stored at −70° C. until further preparation.

Sample Preparation

Brain samples were thawed on ice for 1 hour and homogenates were prepared by adding PBS-buffer (phosphate buffered saline, 0.1 μmol/L; Sigma Aldrich, Vienna, Austria) to tissue sample, ratio 3:1 (w/v), and homogenized with a Potter S homogenizer (Sartorius, Goettingen, Germany) at 9 g on ice for 1 minute. To enable analysis of all samples in one batch, samples were frozen again (−70° C.), thawed on ice (1 h) on the day of analysis and centrifuged at 18000 g at 2° C. for 5 min. All tubes were prepared with 0.001% BHT (butylated hydroxytoluene; Sigma-Aldrich, Vienna, Austria) to prevent autooxidation [Morrow, J. D. and L. J. Roberts. Mass spectrometry of prostanoids: F2-isoprostanes produced by non-cyclooxygenase free radical-catalyzed mechanism. Methods Enzymol. 233 (1994): 163-74].

Overall the data obtained from nine control and seven ischemic animal samples were processed. The metabolite concentrations were measured using a commercial Kit (Marker IDQ™, Biocrates AG, Innsbruck, Austria) as well as other mass-spectroscopy based methods described below.

Extracted samples were analyzed by a new developed online solid phase extraction liquid chromatography tandem mass spectrometry method (online SPE-LC-MS/MS). All procedures (sample handling, analytics) were performed by co-workers blinded to the groups. For simultaneous quantitation of free prostaglandins and lipoxygenase derived fatty acid metabolites in brain homogenates we used a LC-MS/MS based method as described by Unterwurzacher et al. [Unterwurzacher I, Koal T, Bonn G K et al. Rapid sample preparation and simultaneous quantitation of prostaglandins and lipoxygenase derived fatty acid metabolites by liquid chromatography-mass spectrometry from small sample volumes. Clin Chem Lab Med. 2008; 46:1589-1597] for brain tissue. Due to matrix effects observed during analysis of brain samples, an online solid phase extraction (SPE) step was implemented prior to chromatographic separation using a C18 Oasis HLB column (2.1×20 mm, 25 μm particle size; Waters, Vienna, Austria) as online SPE column. The quantification of the metabolites in the extracted biological sample is achieved by reference to appropriate internal standards and by use of the most sensitive and selective electrospray ionization (ESI) multiple reaction monitoring (MRM) MS/MS detection mode. The method was validated for tissue samples homogenates according the “Guidance for Industry—Bioanalytical Method Validation”, U.S. Department of Health and Human Services, Food and Drug Administration, 2001. For the online SPE-LC-MS/MS analysis 20 μL of the extracted homogenate was injected.

RNA Extraction and cDNA Synthesis:

The two divided brain hemispheres of newborn RNU rats were collected in 1 ml TRIzol Reagent (Invitrogen Life Technologies, Austria), frozen in liquid nitrogen and stored at −80° C. until further processing. The RNA extraction was done according to manufacturer's instructions. Briefly, the brain hemispheres were homogenized in TRIzol on ice using a micropistill. After complete homogenization a chloroform extraction step resulting in an RNA containing aqueous phase, followed by precipitation with isopropyl alcohol was affiliated. After two washing steps with 75% ethanol the briefly air dried RNA was resuspended in DEPC-treated water, the RNA concentration was determined using an UV-spectrophotometer (Ultrospec 3300 pro, Amersham, USA) and stored at −80° C. until processing for cDNA synthesis.

Prior to reverse trancription (RT) an amount of 1 μg of total RNA was treated with DNase I, RNase-free (Deoxyribonuclease I, Fermentas, Germany) according to manufacturer's instructions to remove potential contaminating DNA. After DNase I treatment the samples were processed for cDNA synthesis using the RevertAid M-MuLV reverse transcriptase (Fermentas, Germany). Each reaction consisted of 5×RT-reaction buffer, 10 mM deoxyribonucleotide triphosphate mixture (dNTPs), 0.2 μg/μl random hexamer primer, an RNase inhibitor and the RevertAid M-MuLV-RT (all from Fermentas, Germany). Samples were incubated at 25° C. for 10 minutes followed by 60 minutes at 42° C. in a waterbath. The reaction was terminated by heating to 70° C. for 10 minutes followed by chilling on ice. The cDNA samples were stored at −20° C. until processing for quantitative real-time PCR using the BioRad iCycler iQ. The cDNA samples were prediluted 1:10 before used as template for quantitative real-time PCR.

Quantitative Real-Time PCR (q-RT-PCR):

The quantitative real-time PCR was carried out in 96-well 0.2 ml thin-wall PCR plates covered with optically clear adhesive seals (BioRad Laboratories, Austria) in a total volume of 25 μl. The real-time PCR reaction mixture consisted of 1×1Q SYBR Green Supermix (BioRad Laboratories, Austria), 0.4 μM of each gene specific primer and 5 μl of prediluted cDNA. Initially the mixture was heated to 95° C. for 3 minutes to activate the iTaq DNA polymerase, followed by 45 cycles consisting of denaturation at 95° C. for 20 seconds and annealing at 60° C. for 45 seconds. After the amplification a melting curve analysis was added to confirm PCR product specificity. No signals were detected in the no-template controls.

The results were analysed using the iCycler iQ5 Optical System Software Version 2.0 (BioRad Laboratories, Austria). The baseline was manually set and the threshold automatically by the software.

The crossing point of the amplification curve with the threshold line represents the cycle threshold (ct). All samples were run in triplicates and the mean value was used for further calculations.

During the optimization process all gene specific primer pairs were run in a gradient PCR to determine the optimal annealing temperature, the PCR products were loaded on a 2% agarose gel containing ethidium bromide to confirm specificity of the amplification product and the absence of primer dimer formation.

The sequence of gene specific primer pairs used are given in Table 28 (1^{st} column is SEQ-ID-No).

TABLE 28
Table 28: Metabolite data, classification error
via LOO cross validation

| | | | |
|---|---|---|---|
| 901 | rSDF1a-LC1 | 181 bp | 5′-AGTGACGGTAAGCCAGTCAG-3′ |
| 902 | rSDF1a-LC2 | | 5′-TCCACTTTAATTTCGGGTCA-3′ |
| 903 | rVEGF-LC1 | 195 bp | 5′-GAAAGGGAAAGGGTCAAAAA-3′ |
| 904 | rVEGF-LC2 | | 5′-CACATCTGCAAGTACGTTCG-3′ |
| 905 | rACTB-LC1 | 160 bp | 5′-AAGAGCTATGAGCTGCCTGA-3′ |
| 906 | rACTB-LC2 | | 5′-TACGGATGTCAACGTCACAC-3′ |

Analysis of qPCR and Metabolomics Data:

For developing and validating a classifier based on these data we used support vector machines [Schöllkopf, B. and Smola, A. (2001) Learning with Kernels: Support Vector Machines, Regularization, Optimization, and Beyond. MIT Press, Cambridge] in combination with leave-one-out (LOO) cross-validation where each analysis step—including low level analysis—was repeated in each cross-validation step. This is one possibility. Of course, we could also have used a split-sample, a bootstrap or a different k-fold (k not equal to 1) cross-validation approach. Moreover, we could have used a different class of functions for classification e.g. logistic regression, (diagonal) linear or quadratic discriminant analysis (LDA, QDA, DLDA, DQDA), shrunken centroids regularized discriminant analysis (RDA), random forests (RF), support vector machines (SVM), generalized partial least squares (GPLS), partitioning around medoids (PAM), self organizing maps (SOM), recursive partitioning and regression trees, K-nearest neighbor classifiers (K-NN), bagging, boosting, naïve Bayes and many more.

The low level analysis consisted of a variance stabilizing transformation via the binary logarithm (i.e., log to base 2) for the metabolite data In each cross validation step we selected those four normalized metabolites, which had the largest differences (in absolute value) of the mean values beyond those probes with p value equal or smaller than 0.1 by the Welch t-test. This is, we used a so called ranker for feature selection. Again there are numerous other feature selection strategies we could have used, some examples are given in Hall et al. 2003 Overall a metabolite may have been chosen up to 16 times due to LOO cross-validation. Using only metabolomics data we obtain the estimated errors given in Table 29.

TABLE 29
Table 29: Metabolite data, classification error via LOO cross validation

| classifier vs. true | ischemia | control |
|---|---|---|
| Ischemia | 57.1% | 33.3% |
| Control | 42.9% | 66.7% |

The estimated overall accuracy using LOO cross-validation is 62.5%, sensitivity is 57.1%, specificity is 66.7%, positive predictive value is 57.1% and negative predictive value is 66.7%. In a second step we used qPCR data obtained for SDF1 and VEGF. The PCR data was normalized via the reference gene Actin-beta. The classification results can be read off from Table 30. We get an estimated overall accuracy of 68.9% again using LOO cross-validation. The estimated values for sensitivity, specificity, positive and negative predictive value are 57.1%, 77.8%, 66.7% and 70.0%, respectively.

TABLE 30
Table 30: qPCR data, classification error via LOO cross validation

| classifier vs. true | ischemia | normal |
|---|---|---|
| Ischemia | 57.1% | 22.2% |
| Normal | 42.9% | 77.8% |

In the last step we combine metabolite and qPCR data and obtain the results given in Table 31. That is, the estimated overall accuracy using cross-validation is 75.0%. Hence, this combination increases the overall accuracy from 62.5% resp. 68.9% to 75.0%. Sensitivity, specificity, positive and negative predictive value are 71.4%, 77.8%, 71.4% and 77.8%, respectively. Hence, beside overall accuracy, sensitivity as well as positive and negative predictive value are enhanced.

TABLE 31
Table 31: Metabolite and qPCR data, classification error via
LOO cross validation

| classifier vs. true | ischemia | normal |
|---|---|---|
| Ischemia | 71.4% | 22.2% |
| Normal | 18.6% | 77.8% |

The metabolites which were selected during cross-validation are given in Table 32.

TABLE 32
Table 32: Metabolites selected during LOO cross validation

| | | Times | |
|---|---|---|---|
| Nr. | Metabolite | selected | Comments |
| 1 | Gln-PTC | 16 | PTC = Phenylthiocarbamoyl |
| 2 | xLeu-PTC | 15 | |
| 3 | Ala-PTC | 11 | |
| 4 | 12S-HETE | 8 | =12(S)-Hydroxyeicosatetraenoic acid |
| 5 | Alanine | 4 | |
| 6 | xLeucine | 3 | |
| 7 | DHA | 3 | =Decosahexaenoic acid |
| 8 | Ser-PTC | 2 | |
| 9 | Glu | 1 | |
| 10 | Glutamic Acid | 1 | |

In Table 32, the total of times selected must be 64, wherein each individual metabolite might be selected a maximum of 16 times.

TABLE 33
Table 33: Metabolite data, classification error via LOO cross validation
(1^{st} column is SEQ-ID-No)

| | | | |
|---|---|---|---|
| 265 | ACTB | NM_001101.2 | Hs.520640 |
| | | | Hs.708120 |

### EMBODIMENTS OF THE INVENTION

In one embodiment, first, a biological sample from a subject in need of diagnosis, or response or survival prognostication is obtained. Second, an amount of a RNA, microRNA, peptide or protein, metabolite is selected and is measured from the biological sample. Third, the amount of RNA, microRNA, peptide or protein, metabolite, is detected in the sample and is compared to either a standard amount of the respective biomolecule present in a normal cell or a non-cancerous cell or tissue or plasma, or an amount of the RNA, microRNA, peptide or protein, metabolite is present in the control sample. If the amount of RNA, microRNA, peptide or protein, metabolite in the sample is different to the amount of RNA, microRNA, peptide or protein, metabolite in the standard or control sample, the processing and classification of concentration data and classifier generation as described before (Table 1) from at least two groups/species of biomolecules comprising RNA, microRNA, peptide or protein, metabolites affords a value or score assigned to a diseased state with some probability then the subject is diagnosed as having cancer, the prognosis is a low expected response to the cancer treatment, or the prognosis is a low expected survival of the subject. The prognoses are relative to a subject with cancer having normal levels of the RNA, microRNA, peptide or protein, metabolite or relative to the average expected response or survival of a patient having a complex disease. It is clear that these complex diseased states can also be due to intoxication and drug abuse.

Another embodiment of the method of detecting or diagnosing a complex disease, prognosticating an expected response to a, or prognosticating an expected survival comprises the following steps. First, a biological sample containing RNA, microRNA, peptide or protein, metabolite is obtained from the subject. The biological sample is reacted with a reagent capable of binding to an RNA, microRNA, peptide or protein, metabolite. The reaction between the reagent and the microRNA forms a measurable RNA, microRNA, peptide or protein, metabolite product or complex. The measurable RNA, microRNA, peptide or protein, metabolite product or complex is measured, the data processed to afford a score applying the steps as specified under FIG. 1 and then compared to either the standard or the control score value.

The examples indicate that the method according to the invention includes the analysis and classifier generation from quantitative data of the aforementioned types of biomolecules obtained from different, distinct tissues from one individual and show that this is advantageous in recognizing distinct states related to complex diseases as data from different sites of an affected organism contribute to biomarker/classifier description.

The invention can be practiced on any mammalian subject including humans, that has any risk of developing a complex disease in the sense of the present invention.

Samples to be used in the invention can be obtained in any manner known to a skilled artisan. The sample optimally can include tissue believed to be cancerous, such as a portion of a surgically removed tumor but also blood containing cancer cells. However, the invention is not limited to just tissue believed to be altered (with regard to concentrations of biomolecules such as RNA, micro RNA, protein, peptide, metabolites) due to a complex disease. Instead, samples can be derived from any part of the subject containing at least some tissue or cells believed to be affected by the complex disease, in particular, cancer and/or having being exposed or in contact to cancer tissue or cells or by contact to body liquids such as blood distributing certain biomolecules within the body.

Another example of a method of quantifying RNA or microRNA is as follows: hybridizing at least a portion of the RNA or microRNA with a fluorescent nucleic acid, and reacting the hybridized RNA or microRNA with a fluorescent reagent, wherein the hybridized RNA or microRNA emits a fluorescent light. Another method of quantifying the amount of RNA or microRNA in a sample is by hybridizing at least a portion the RNA or microRNA to a radio-labeled complementary nucleic acid. In instances when a nucleic acid capable of hybridizing to the RNA or microRNA is used in the measuring step, in case of the microRNA the nucleic acid is at least 5 nucleotides, at least 10 nucleotides, at least 15 nucleotides, at least 20 nucleotides, at least 25 nucleotides, at least 30 nucleotides or at least 40 nucleotides; and may be no longer than 25 nucleotides, no longer than 35 nucleotides; no longer than 50 nucleotides; no longer than 75 nucleotides, no longer than 100 nucleotides or no longer than 125 nucleotides in length. The nucleic acid is any nucleic acid having at least 80% homology, 85% homology, 90% homology, 95% homology or 100% homology with any of the complementary sequences for the microRNAs. A suitable RNA parameter, e.g. is the amount of RNA or microRNA which is compared to either a standard amount of the RNA or microRNA present in a normal cell or a non-cancerous cell, or to the amount of RNA or microRNA in a control sample. The comparison can be done by any method known to a skilled artisan. An example of comparing the amount of the RNA or microRNA in a sample to a standard amount is comparing the ratio between 5S rRNA and the RNA or microRNA in a sample to a published or known ratio between 5S rRNA and the RNA or microRNA in a normal cell or a non-cancerous cell. An example of comparing the amount of microRNA in a sample to a control is by comparing the ratios between 5 S rRNA and the RNA or microRNA found in the sample and in the control sample. In instances when the amount of RNA or microRNA is compared to a control, the control sample may be obtained from any source known to have normal cells or non-cancerous cells. Preferably, the control sample is tissue or body fluid from the subject believed to be unaffected by the respective complex disease contain only normal cells or non-cancerous cells.

Measuring the amount of RNA, microRNA, peptide or protein, metabolite can be performed in any manner known by one skilled in the art of measuring the quantity of RNA, microRNA, peptide or protein within a sample. An example of a method for quantifying RNA or microRNA is quantitative reverse transcriptase polymerase chain reaction, PCR or quantitation and relative quantitation applying sequencing or second generation sequencing.

Protein measurement, absolute and relative protein quantitation of individual protein species as well as quantitation of metabolites within a tissue or in a preparation of cells can be performed applying Western blotting, Enzyme Linked Immunoassay (ELISA) Radio-immunoassay or other assays utilizing antibodies or other protein binding molecules, mass spectrometry for protein or peptide identification, quantitation or relative quantitation using MALDI, Electrospray or other types of ionisation, protein and antibody arrays employing antibodies or other molecules binding proteins such as aptamers. The compound capable of binding to RNA, microRNA, peptide or protein and metabolite can be any compound known to a skilled artisan as being able to bind to the RNA, microRNA, peptide or protein in a manner that enables one to detect the presence and the amount of the molecule. An example of a compound capable of binding RNA, microRNA, peptides or proteins as well as low molecular weight compounds and metabolites is a nucleic acid capable of hybridizing or an aptamer capable of binding to nucleic acids, RNA, microRNA, proteins and peptides. The nucleic acid preferably has at least 5 nucleotides, at least 10 nucleotides, at least 15 nucleotides, at least 20 nucleotides, at least 25 nucleotides, at least 30 nucleotides, at least 40 nucleotides or at least 50 nucleotides. The nucleic acid is any nucleic acid having preferably at least 80% homology, 85% homology, 90% homology, 95% homology or 100% homology with a sequence complementary to an RNA or microRNA, which also might be derived from corresponding DNA data or an aptamer capable of binding RNA, microRNA, peptide or protein or metabolite. One specific example of a nucleic acid capable of binding to RNA or microRNA is a nucleic acid primer for use in a reverse transcriptase polymerase chain reaction.

The binding of the compound to at least a portion of the RNA, microRNA, peptide or protein and metabolite forms a measurable complex. The measurable complex is measured according to methods known to a skilled artisan. Examples of such methods include the methods used to measure the amount of the RNA, microRNA, peptide or protein, metabolite employed in the inventive method discussed above.

If there is an increased or decreased level of measurable complex relative to a standard amount of RNA, microRNA, peptide or protein found in a normal or a non-cancerous cell, or in a control sample, then the sample either contains a pre-cancerous cell or cancer cell, thereby being diagnostic of a cancer; prognosticates an expected response to a cancer treatment; or prognosticates an expected survival of the subject.

The inventive composition of the different types of biomolecules can be used in the inventive method (embodiments of which are described above). One embodiment of the inventive composition comprises a compound capable of binding to at least a portion of RNA, microRNA, peptide, protein or metabolite selected from the group consisting of RNA, microRNA, peptide or protein, metabolite. The composition comprises a compound capable of binding to at least a portion of a RNA, microRNA, peptide or protein selected from the group consisting of molecules summarized in the described examples and the lists of molecules and binding probes binding these endogenous biomolecules but is not limited to that. The various examples described above demonstrate that the method generally functions with a composition of 2-4 types of the defined biomolecules, proteins or peptides, RNA, microRNA (i.e. RNA plus microRNA, RNA plus protein, protein plus microRNA, RNA plus protein plus microRNA, and a combination of these biomolecules and combinations of biomolecules with metabolites, selected and combined from various experiments investigating tissue from a subject having a complex disease with a performance which is superior than that of a test or diagnostic or prognostic tool comprising a set of preselected biomolecules composed of just one type such as RNA, protein, metabolite or microRNA solely.

Another embodiment of the inventive composition is a composition comprising a second compound capable of binding to a RNA, microRNA, peptide or protein and metabolite that is different from the RNA, microRNA, peptide or protein, metabolite that the first compound is capable of binding. Another embodiment of the inventive composition is a composition comprising a third compound capable of binding to a RNA, microRNA, peptide or protein, metabolite that is different from the RNA, microRNA, peptide or protein, metabolite that the first and second compounds are capable of binding.

The present invention further provides a method for evaluating candidate therapeutic agents. The method can be applied to identify molecules that modulate the concentrations of one to several of the mentioned biomolecules assigned to at least two or more of the stated molecule classes; RNA, microRNA, peptide/proteins, metabolites. Alternatively, assays may be conducted to identify molecules that modulate the activity of a protein encoded by a gene.

Another aspect of the invention is a kit for diagnosing, or prognosticating a complex disease. In one embodiment of this aspect, the kit is for diagnosing a subject with a complex disease. Another embodiment of this aspect is a kit for prognosticating a a complex disease, wherein the prognosis is an expected response by a subject to a treatment of the a complex disease. In another embodiment of this aspect, the kit is for prognosticating a a complex disease, wherein the prognosis is an expected survival of a subject with a complex disease. The kit comprises a composition capable of binding to at least a portion of a RNA, microRNA, peptide or protein, metabolite with increased or decreased concentration, over- or under-expressed in a cancer cell, wherein the RNA, microRNA, peptide or protein, metabolite is selected from-but not limited to the group consisting of the molecules listed in the examples outlined above or binding to the binding probes or determined quantitatively by methods described in the examples above and wherein the differential expression (over-expression or under-expression or the concentration changes of several molecules out of RNA, microRNA, peptide or protein, metabolites in a combination of at least molecules from 2 different biomolecule classes (RNA plus microRNA, RNA plus proteins or peptides, microRNA plus protein or peptides, RNA plus microRNA plus proteins or peptides and combinations of all these with metabolites), comprising, but not limited, to the classes of compounds, the described binding probes, the agents and sequences specified in the described examples is diagnostic for a complex disease, or prognosticates the expected response or survival of the subject. The binding of the nucleic acid or aptamer or antibody to the target RNA, microRNA, peptide or protein, and or metabolite is diagnostic for a complex disease, prognosticates an expected response to a treatment, or prognosticates an expected survival of a subject having a complex disease.

The isolated RNA, microRNA, peptide or protein, metabolite can be associated with known diagnostic tools, such as protein chips, antibody chips, aptamer chips, DNA or RNA chips with various modes of detection of binding including but not limited to detection by use of fluorophores, electrochemical detection or transfer of an chemical signal to a change of electrical current, resistance or charge, RNA probes, or RNA primers.

One aspect of the invention is a method of detecting for early diagnosing a complex disease, prognosticating an expected response to a treatment, or prognosticating an expected survival.

The present invention finds use with complex diseases, cancer, in a special embodiment with Leukemia (AML), prostate and kidney cancer as well as transient ischemic attack, hypoxia/ischemia. However, as evident already from these distinct and unrelated diseases and diverse types of cancer, diseases with completely different molecular etiology, phenotypes, genotypes and genetic dispositions, the method is applicable to complex diseases in general.

In a specific embodiment, data obtained from different types of biomolecules from different compartments (tissues) of the organism (subject, patient) are used and processed together according to the method thus providing improved classification and diagnosis of complex diseases.

The above descriptions are illustrative and not restrictive. It is to be understood that this invention is not limited to particular methods, and experimental conditions described, as such methods and conditions may vary.

The sequence listing accompanying the present application comprising sequences with SEQ-IDs No 1 to is SEQ-IDs No 908 is part of the disclosure of the present invention.

1. A method for in vitro diagnosing a complex disease or subtypes thereof, selected from the group consisting of:
cancer, in particular, acute myeloid leukemia (AML), colon cancer, kidney cancer, prostate cancer; transient ischemic attack (TIA), ischemia, in particular stroke, hypoxia, hypoxic-ischemic encephalopathy, perinatal brain damage, hypoxic-ischemic encephalopathy of neotatals asphyxia; demyelinating disease, in particular, white-matter disease, periventricular leukoencephalopathy, multiple sclerosis, Alzheimer and Parkinson's disease;
in at least one biological sample of at least one tissue of a mammalian subject comprising:
a) selecting at least two different species of biomolecules, wherein said species of biomolecules are selected from the group consisting of: RNA and/or its DNA counterparts, microRNA and/or its DNA counterparts, peptides, proteins, and metabolites;
b) measuring at least one parameter selected from the group consisting of presence or absence, qualitative and/or quantitative molecular pattern and/or molecular signature, level, amount, concentration and expression level of a plurality of biomolecules of each species in said sample using at least two sets of different species of biomolecules and storing the obtained set of values as raw data in a database;
c) mathematically preprocessing said raw data in order to reduce technical errors being inherent to the measuring procedures used in b);
d) selecting at least one suitable classifying algorithm from the group consisting of logistic regression, (diagonal) linear or quadratic discriminant analysis (LDA, QDA, DLDA, DQDA), perceptron, shrunken centroids regularized discriminant analysis (RDA), random forests (RF), neural networks (NN), Bayesian networks, hidden Markov models, support vector machines (SVM), generalized partial least squares (GPLS), partitioning around medoids (PAM), self organizing maps (SOM), recursive partitioning and regression trees, K-nearest neighbor classifiers (K-NN), fuzzy classifiers, bagging, boosting, and naive Bayes; and applying said selected classifier algorithm to said preprocessed data of c);
e) said classifier algorithms of d) being trained on at least one training data set containing preprocessed data from subjects being divided into classes according to their pathophysiological, physiological, prognostic, or responder conditions, in order to select a classifier function to map said preprocessed data to said conditions;
f) applying said trained classifier algorithms of e) to a preprocessed data set of a subject with unknown pathophysiological, physiological, prognostic, or responder condition, and using the trained classifier algorithms to predict the class label of said data set in order to diagnose the condition of the subject.

2. Method according to claim 1, wherein said tissue is selected from the group consisting of blood and other body fluids, cerebrospinal fluids, bone tissue, bone marrow tissue, muscular tissue, glandular tissue, brain tissue, nerve tissue, mucous tissue, connective tissue, and skin tissue and/or said sample is a biopsy sample and/or said mammalian subject includes humans; and/or
wherein standard lab parameters commonly used in clinical chemistry, such as serum and/or plasma levels of low molecular weight biochemical compounds, enzymes, enzymatic activities, cell surface receptors and/or cell counts, in particular red and/or white cell counts, platelet counts, are additionally selected.

3. Method according to claim 1, wherein said mathematically preprocessing of said raw data obtained in b) is carried out by a statistical method selected from the group consisting of:
in case of raw data obtained by optical spectroscopy (UV, visible, IR, Fluorescence): background correction and/or normalization;
in case of raw data obtained from metabolomics and/or proteomics obtained by mass spectroscopy coupled to liquid or gas chromatography or capillary electrophoresis or by 2D gel electrophoresis, quantitative determination with ELISA or RIA or determination of concentrations/amounts by quantitation of immunoblots or quantitation of amounts of biomolecules bound to aptamers: smoothing, baseline correction, peak picking, optionally, additional further data transformation such as taking the logarithm in order to carry out a stabilization of the variances;
in case of raw data obtained from transcriptomics: Summarizing single pixel to a single intensity signal, background correction; summarizing of multiple probe signals to a single expression value, in particular perfect match/mismatch probes; normalization.

4. Method according to claim 1, wherein after preprocessing in c) a further step of feature selection is inserted, in order to find a lower dimensional subset of features with the highest discriminatory power between classes; and
said feature selection is carried out by a filter and/or a wrapper approach;
wherein said filter approach includes rankers and/or feature subset evaluation methods.

5. Method according to claim 1, wherein said pathophysiological condition corresponds to the label “diseased” and said physiological condition corresponds to the label “healthy” or said pathophysiological condition corresponds to different labels of “grades of a disease”, “subtypes of a disease”, different values of a “score for a defined disease”; said prognostic condition corresponds to a label “good”, “medium”, “poor”, or “therapeutically responding” or “therapeutically non-responding” or “therapeutically poor responding”.

6. Method according to claim 1, wherein said metabolic data is high-throughput mass spectrometry data.

7. Method according to claim 1, wherein said complex disease is AML, said mammalian subject is a human being, said biological sample blood and/or blood cells and/or bone marrow, wherein said different species of biomolecules are microRNA and proteins, in particular surface proteins from non-mature hematopoietic stem cells, preferably CD34;
wherein microRNA expression levels and CD34 presence are used as said parameters of b);
wherein raw data of microRNA expression are preprocessed using a variance-stabilizing normalization and summarizing the normalized multiple probe signals (technical replicates) to a single expression value, using the median;
wherein a ranker, in particular a Mann-Whitney significance test combined with largest median of pairwise differences as filter for microRNA expression data is used for said feature selection;
wherein logistic regression is selected as suitable classifying algorithm, the training of the classifying algorithm including preprocessed and filtered microRNA expression data and CD34 information, is carried out with an n-fold cross-validation, in particular 5 to 10-fold, preferably 5-fold cross-validation;
applying said trained logistic regression classifier to said preprocessed microRNA expression data set and CD34 information to a subject under suspicion of having AML, and using the trained classifiers to diagnose a specific AML-type.

8. Method according to claim 7, wherein the following DNA probes for targeting said microRNA are used: SEQ ID NO: 1 to SEQ ID NO: 14;
and/or
the following microRNA-target sequences are used: SEQ ID NOs: 15 to 26.

9. Method according to claim 1, wherein said complex disease is colon cancer, said mammalian subject is a human being, said biological sample is colon tissue;
wherein said different species of biomolecules are mRNA and/or its DNA counterparts and microRNA and/or its DNA counterparts;
wherein mRNA expression levels and microRNA expression levels are used as said parameters of b);
wherein raw data of microRNA expression are preprocessed using a variance stabilizing normalization;
wherein raw data of mRNA expression are preprocessed using a variance stabilizing normalization and summarizing the perfect match (PM) and miss match (MM) probes to an expression measure using a robust multi-array average (RMA);
wherein a ranker, in particular a Mann-Whitney significance test combined with largest median of pairwise differences as filter for microRNA expression data is used for said feature selection;
wherein random forests are selected as suitable classifying algorithm, the training of the classifying algorithm including preprocessed and filtered mRNA and microRNA expression data, is carried out with a leave-one-out (LOO) cross-validation,
applying said trained random forests classifier to said preprocessed mRNA and microRNA expression data sets to a subject under suspicion of having colon cancer, and using the trained classifiers to diagnose colon cancer and/or a subtype thereof.

10. Method according to claim 9, wherein the following DNA probes for targeting said microRNA are used: SEQ ID NO:27 to SEQ ID NO: 34;
and/or
the following microRNA-target sequences are used: SEQ ID NO:35 to SEQ ID NO:42;
and/or
the following DNA probes for targeting said mRNA′ are used: SEQ ID NO:43 to SEQ ID NO:264;
and/or
the following target DNA sequences are used: SEQ ID NO:265 to 276.

11. Method according to claim 1, wherein said complex disease is kidney cancer, said mammalian subject is a human being, said biological sample is kidney tissue;
wherein said different species of biomolecules are mRNA and/or its DNA counterparts and microRNA and/or its DNA counterparts;
wherein mRNA expression levels and microRNA expression levels are used as said parameters of b);
wherein raw data of microRNA expression are preprocessed using a variance-stabilizing normalization;
wherein raw data of mRNA expression are preprocessed using a variance stabilizing normalization and summarizing the perfect match (PM) and miss match (MM) probes to an expression measure using a robust multi-array average (RMA);
wherein a ranker, in particular a Welch t-test (significance test) combined with largest mean of pairwise differences as filter for mRNA and microRNA expression data is used for said feature selection;
wherein single-hidden-layer neural networks are selected as suitable classifying algorithm, the training of the classifying algorithm including preprocessed and filtered mRNA and microRNA expression data, is carried out with a leave-one-out (LOO) cross-validation; applying said trained single-hidden-layer neural networks classifier to said preprocessed mRNA and microRNA expression data sets to a subject under suspicion of having kidney cancer, and using the trained classifiers to diagnose kidney cancer and/or a subtype thereof.

12. Method according to claim 11, wherein the following DNA probes for targeting said microRNA are used: SEQ ID NOs:33, and 277 to 288;
and/or
the following microRNA-target sequences are used: SEQ ID NOs:21, 41, 289 to 297;
and/or
the following DNA probes for targeting said mRNA are used: SEQ ID NOs: 298 to 716;
and/or
the following DNA target sequences are used: SEQ ID NOs:265, 268, 717 to 732.

13. Method according to claim 1, wherein said complex disease is prostate cancer, said mammalian subject is a human being, said biological sample is urine and/or prostate tissue;
wherein said different species of biomolecules are mRNA and/or its DNA counterparts and microRNA and/or its DNA counterparts;
wherein mRNA expression levels and mirrnRNA expression levels are used as said parameters of step b);
wherein raw data of microRNA expression are preprocessed using a variance stabilizing normalization;
wherein raw data of mRNA expression are preprocessed using a variance-stabilizing normalization and summarizing the perfect match (PM) and miss match (MM) probes to an expression measure using a robust multi-array average (RMA);
wherein a ranker, in particular a Mann-Whitney significance test combined with largest median of pairwise differences as filter for mRNA and microRNA expression data is used for said feature selection;
wherein linear discriminant analysis is selected as suitable classifying algorithm, the training of the classifying algorithm including preprocessed and filtered mRNA and microRNA expression data, is carried out with a leave-one-out (LOO) cross-validation;
applying said trained linear discriminant analysis classifier to said preprocessed mRNA and microRNA expression data sets to a subject under suspicion of having prostate cancer, and using the trained classifiers to diagnose prostate cancer and/or a subtype thereof.

14. Method according to claim 13, wherein the following DNA probes for targeting said microRNA are used: SEQ ID NOs:733 to 735;
and/or
the following microRNA-target sequences are used: SEQ ID NOs:736-738;
and/or
the following DNA probes for targeting said mRNA are, used: SEQ ID NO:739 to SEQ ID NO:892;
and/or
the following DNA target sequences are used: SEQ ID NOs:893 to 900.

15. Method according to claim 1, wherein said complex disease is transient ischemic attack (TIA) and/or ischemia and/or hypoxia, said mammalian subject is a human being, said biological sample blood and/or blood cells and/or cerebrospinal fluid and/or brain tissue;
wherein said different species of biomolecules are mRNA and/or its DNA counterparts and brain metabolites, in particular free prostaglandins, lipoxygenase derived fatty acid metabolites, glutamine, glutamic acid, leucin, alanine, serine, decosahexaenoic acid (DHA), 12(S)-hydroxyeicosatetraenoic acid (12S-HETE);
wherein mRNA expression levels and quantitative and/or qualitative molecular metabolite patterns (metabolomics data) are used as said parameters of step b);
wherein raw data of mRNA expression are preprocessed using actin-β as reference genes and metabolomics data of said brain metabolites are preprocessed by a variance stabilizing transformation via the binary logarithm (i.e. to base 2);
wherein a ranker, in particular a Welch t-test (significance test) combined with largest mean of pairwise differences as filter for metabolomics data is used for said feature selection;
wherein support vector machines are selected as suitable classifying algorithm, the training of the classifying algorithm including preprocessed and filtered mRNA and microRNA expression data, is carried out with a leave-one-out (LOO) cross-validation;
applying said trained support vector machines classifier to said preprocessed mRNA expression data and said metabolomics data sets to a subject under suspicion of having ischemia and/or hypoxia, and using the trained classifiers to diagnose ischemia and/or hypoxia and/or the grades thereof.

16. Method according to claim 15, wherein the samples are analyzed by solid phase extraction liquid chromatography tandem mass spectrometry (online SPE-LC-MS/MS), wherein preferably a C18 column is used as solid phase extraction column; and wherein the quantification of the measured metabolite concentrations in said biological tissue sample preferably is calibrated by reference to internal standards and by using an electrospray ionization multiple reaction monitoring tandem mass spectrometry detection mode.

17. Method according to claim 15, wherein the mRNA expression data are obtained by quantitative real time PCR (q-RT-PCR);
and/or
the following primer pairs are used: SEQ ID NOs:901 to 906; and/or the following DNA target sequences are used: SEQ ID NOs:265, 907 and 908.

18. Kit for carrying out a method in accordance with claim 1, in a biological sample, comprising:
a) detection agents for the detection of at least two different species of biomolecules, wherein said species of biomolecules are selected from the group consisting of: RNA and/or its DNA counterparts, microRNA and/or its DNA counterparts, peptides, proteins, and metabolites;
b) positive and/or negative controls; and
c) classification software for classification of the results achieved with said detection agents.
